(19) United States
(12) Patent Application Publication
(10) Pub. No.: US 2024/0049994 A1
(43) Pub. Date: Feb. 15, 2024

(54) ONE-TOUCH FINGERTIP SWEAT SENSOR AND PERSONALIZED DATA PROCESSING FOR RELIABLE PREDICTION OF BLOOD BIOMARKER CONCENTRATIONS

(71) Applicant: THE REGENTS OF THE UNIVERSITY OF CALIFORNIA, Oakland, CA (US)

(72) Inventors: Lu Yin, La Jolla, CA (US); Hazhir Teymourian, La Jolla, CA (US); Joseph Wang, San Diego, CA (US); Juliane R. Sempionatto-Moreto, Pasadena, CA (US); Jong-Min Moon, San Diego, CA (US)

(21) Appl. No.: 18/264,755

(22) PCT Filed: Feb. 7, 2022
(86) PCT No.: PCT/US2022/070554

§ 371 Date: Aug. 8, 2023

Related U.S. Application Data
(60) Provisional application No. 63/182,579, filed on Apr. 30, 2021.
(60) Provisional application No. 63/146,359, filed on Feb. 5, 2021.

(51) Int. Cl. A61B 5/145

(52) U.S. Cl. CPC A61B 5/14517; A61B 5/14546; A61B 2562/0214

(57) ABSTRACT

Methods, systems, and devices are disclosed for collecting and transferring naturally-produced sweat containing an analyte to a biosensor and/or biofuel cell to estimate a concentration of the analyte corresponding to the analyte's concentration in blood and/or for producing electricity. In some aspects, a device includes a substrate, a plurality of electrodes disposed on the substrate and operable to detect an analyte in naturally-produced sweat of an individual, and a sweat permeation layer including a hydrogel, wherein the sweat permeation layer is in contact with the plurality of electrodes and configured to transfer the sweat containing the analyte through the sweat permeation layer to reach the plurality of electrodes for detection and/or energy harvesting.

### PRIORITY CLAIM AND CROSS-REFERENCE TO RELATED APPLICATION

This patent document claims priorities and benefits of U.S. Provisional Application No. 63/146,359 filed on Feb. 5, 2021 and U.S. Provisional Application No. 63/182,579 filed on Apr. 30, 2021. The disclosures of the above patent applications are incorporated by reference as part of the disclosure of this document.

### TECHNICAL FIELD

This patent document relates to electrochemical sensors.

### BACKGROUND

Diabetes prevalence has been rising exponentially, increasing the need for reliable non-invasive approaches to glucose monitoring. Different biofluids have been explored recently for replacing current blood fingerstick glucose strips with non-invasive painless sensing devices. While sweat has received considerable attention, there are mixed reports on correlating the results of sweat-based analysis with blood glucose levels. Therefore, the need still exists to provide simple, inexpensive, and reliable devices and methods for reliable non-invasive measurements of blood glucose as well as other biomarkers based on sweat analysis.

### SUMMARY

The technology disclosed in this patent document relates methods and devices for collecting an analyte in sweat to estimate a concentration of the analyte in blood or for producing electricity by using a redox reaction of the analyte in sweat.

In some aspects, the disclosed technology can be implemented to provide a device that includes a substrate; a plurality of electrodes disposed on the substrate and operable to detect an analyte in sweat of an individual; and a sweat permeation layer including a hydrogel and having a first side and a second side located opposite to the first side, wherein the first side of the sweat permeation layer is in contact with the plurality of electrodes such that the plurality of electrodes is disposed between the substrate and the first side of the sweat permeation layer, wherein the sweat permeation layer is configured to transfer the sweat containing the analyte that is naturally produced from the individual's fingertip by permeating the naturally produced sweat through the sweat permeation layer from the second side to the first side to reach the plurality of electrodes.

In some aspects, the disclosed technology can be implemented to provide a device that includes a piezoelectric chip; two or more electrodes including an anode electrode and a cathode electrode formed over the piezoelectric chip and operable to detect an electrical signal associated with a chemical reaction involving an analyte contained in sweat of an individual incident in a region at a surface of the anode electrode and the cathode electrode; a current collector including two or more electrically-conductive material structures disposed between the piezoelectric chip and the two or more electrodes to electrically couple at least one of the electrically-conductive material structures to the anode electrode and at least another one of the electrically-conductive material structures to the cathode electrode; and a sweat permeation layer including a hydrogel and having a first side and a second side located opposite to the first side, wherein the first side of the sweat permeation layer is in contact with the two or more electrodes and configured to transfer the sweat that is naturally produced from the individual's fingertip by permeating the naturally produced sweat through the sweat permeation layer from the second side to be pressed by the individual's fingertip to the first side to reach the region at the surface of the two or more electrodes, wherein the piezoelectric chip undergoes a non-destructive mechanical deformation upon pressing the second side of the sweat permeation layer with the individual's fingertip, generating electrical energy from the non-destructive mechanical deformation of the piezoelectric chip.

In some aspects, the disclosed technology can be implemented to provide a method for determining a concentration of an analyte present in at least one of blood, sweat, or interstitial fluid (ISF) of an individual, comprising obtaining sample of sweat by the device from deposition of the sample of sweat onto the sweat permeation layer of the device from a finger or other sweat-gland covered skin surfaces of the individual, acquiring a plurality of measurements of a level of the analyte using a signal from the device, obtaining, for each of the plurality of measurements of the level of the analyte, a measurement of a concentration of the analyte in blood of the individual, obtaining a linear slope parameter and an intercept parameter for a dependence between the obtained measurements of the concentration of the analyte in blood of the individual and the obtained measurements of the level of the analyte in sweat of the individual, and using the linear slope parameter and the intercept parameter to translate a new measurement of the level of the analyte in sweat of the individual to an estimate of the concentration of the analyte in blood of the individual.

In some aspects, the disclosed technology can be implemented to provide a method for determining a concentration of an analyte present in at least one of blood, sweat, or interstitial fluid (ISF) of an individual, comprising obtaining sample of sweat by the device from deposition of the sample of sweat onto the sweat permeation layer of the device from a finger of the individual, acquiring a plurality of measurements of a level of the analyte using a signal from the device, obtaining, for each of the plurality of measurements of the level of the analyte, a measurement of a concentration of the analyte in blood of the individual, obtaining an exponential power parameter, an exponential multiplier parameter, and an intercept parameter for a dependence between the obtained measurements of the concentration of the analyte in blood of the individual and the obtained measurements of the level of the analyte in sweat of the individual, and using the exponential power parameter, the exponential multiplier parameter, and the intercept parameter to translate a new measurement of the level of the analyte in sweat of the individual to an estimate of the concentration of the analyte in blood of the individual.

In some aspects, the disclosed technology can be implemented to provide a method for determining a concentration of an analyte in blood of an individual, comprising obtaining sample of sweat by the device from deposition of the sample of sweat onto the sweat permeation layer of the device from a finger of the individual, acquiring a plurality of groups of measurements of a level of the analyte in sweat of the individual using a signal from the device, obtaining, for each group of measurements of the level of the analyte in sweat of the individual, a corresponding group of measurements of a concentration of the analyte in blood of the individual, obtaining, for each group of measurements of the level of the analyte in sweat of the individual, values of a linear slope parameter and an intercept parameter for a dependence between the measurements in the group and the measurements in the corresponding group of measurements of the concentration of the analyte in blood of the individual, determining an average value of the linear slope parameter and an average value of the intercept parameter for the groups of measurements of the level of the analyte in sweat of the individual, and determining a concentration of the analyte in blood of the individual based on the determined average value of the linear slope parameter and the determined average value of the intercept parameter.

In some aspects, the disclosed technology can be implemented to provide a method for generating power using a sweat analyte, comprising: placing the device on a skin surface with sweat glands to collect the sweat analyte for biocatalytic reaction in the plurality of electrodes to generate a current from the plurality of electrodes of the device, wherein the sweat is collected by the device from a finger of a sweat-gland covered skin through the sweat permeation layer of the device, and sporadically applying pressure to the device against the skin via finger pressing to generate a current from the plurality of electrodes, collecting an energy directly within highly porous electrodes of the device or through a voltage regulatory circuit to a storage unit.

In some aspects, the disclosed technology can be implemented to provide a method for determining a concentration of a biofluid analyte of an individual, comprising obtaining sample of sweat by the device from deposition of the sample of sweat onto the sweat permeation layer of the device from a finger of the individual, acquiring a plurality of measurements of a level of the biofluid analyte in sweat of the individual using a self-generated signal or open-circuit voltage from the device, obtaining, for each of the plurality of measurements of the level of the biofluid analyte in sweat of the individual, a voltage signal without external exertion of a constant voltage or current by discharging via a resistive load between an anode and a cathode of the plurality of electrodes, and discharging, for each of the plurality of measurements of the level of the biofluid analyte in sweat of the individual, from a biofuel cell of the device, power that is regulated or stored to power electronics that obtain the signal from the plurality of electrodes.

The above and other aspects and implementations of the disclosed technology are described in more detail in the drawings, the description and the claims.

### BRIEF DESCRIPTION OF THE DRAWINGS

FIGS. 1A-1F show an example of a bloodless fingerstick sweat glucose sensor based on some embodiments of the disclosed technology.

FIGS. 2A-2D show an example of data processing protocol for personalized transduction equation.

FIGS. 3A-3F show examples of effects of different data transformation steps that translate current response of the sweat glucose sensor to the sweat-based blood glucose concentration.

FIGS. 4A-4C show examples of measurement results obtained for whole day sweat glucose measurements along with the corresponding blood measurements.

FIGS. 5A-5B show example principles of operation of a fingertip levodopa biosensor based on some embodiments of the disclosed technology.

FIGS. 6A-6C show an example timeline followed during Levodopa monitoring in sweat.

FIG. 7 shows example data collected during an example on-body demonstration of the Levodopa biosensor.

FIG. 8 shows example comparisons of Levodopa profiles obtained via Levodopa measurements in blood and sweat.

FIGS. 9A-9C show in vitro calibration curve for the glucose sensor.

FIGS. 10A-10E show an optimization of the hand washing step with three repeated experiments using different glucose sensor.

FIGS. 11A-11F show an optimization of the touching time.

FIGS. 12A-12B show a stability of the personal factors

FIG. 13 shows a flowchart for the calibration and analysis of sweat glucose signals to blood glucose concentrations using the fingertip touch-based sensor.

FIG. 14 shows box plots for mean absolute relative difference (MARD) on successive measurements during the day for three subjects.

FIGS. 15A-15B show an example data processing protocol for personalized transduction equation.

FIGS. 16A-16C show a whole day sweat glucose determination.

FIG. 17 shows an application of the fingertip sweat sensor.

FIGS. 18A-18F show an example of molecular imprinted polymer (MIP)-based sensor for rapid, stressless cortisol sensing.

FIGS. 19A-19N show an optimization and calibration of the MIP cortisol sensing in various media.

FIGS. 20A-20F show an example of endogenous cortisol monitoring.

FIGS. 21A-21F show an example of cortisol sensing during acute stimulation via CPT.

FIGS. 22A-22E show an example of on-body cortisol detection using the wearable sensor patch.

FIGS. 23A-23E show diagrams and data plots depicting example embodiments of and implementations for operation of a touch-based biofuel cell (BFC) and bioenergy harvesting system in accordance with the present technology.

FIG. 24 shows data from an example in-vitro and in-vivo characterization implementation of the example touch-based BFC and bioenergy harvesting system.

FIG. 25 shows data from an example optimization implementation for BFC usage patterns of the example touch-based BFC and bioenergy harvesting system.

FIG. 26 shows data from an example performance implementation of the touch-based BFC and the integrated harvesting system.

FIGS. 27A-27G show diagrams and data plots depicting example embodiments of and implementations for operation of self-powered sensor-display system in accordance with the present technology.

FIG. 28 shows a synthesis of carbon nanotube (CNT) foam.

FIG. 29 shows a photographic image of bending a strip of 1×3 cm2 CNT foam.

FIG. 30 shows a water wicking performance of the CNT foam.

FIG. 31 shows an assembly of the CNT foam for BFC and lead zirconate titanate (PZT) chips.

FIG. 32 shows scanning electron microscopy (SEM) images and corresponding electron dispersive X-ray spectroscopy (EDS) mapping of the CNT foam cathode.

FIG. 33 shows cryogenic scanning electron microscopy (cryo-SEM) images of the cross-sections of the porous and non-porous polyvinyl alcohol (PVA) hydrogels.

FIG. 34 shows BFC anode to cathode area ratio optimization.

FIG. 35 shows linear scan voltammetry (LSV) characterization of the cathode with different electrode materials.

FIG. 36 shows LSV characterization of the anode without and with 15 mM of lactate.

FIG. 37 shows LSV response of the BFC after area ratio optimization.

FIG. 38 shows electrochemical impedance spectroscopy (EIS) Nyquist plot of the 2-electrode biofuel cell (BFC) covered by the porous PVA hydrogel with different applied pressure.

FIG. 39 shows optical microscopic images of the finger with applied bromophenol dye.

FIG. 40 shows BFC performance with subjects with different natural fingertip sweat rates.

FIG. 41 shows hydrogel stability in extended harvesting tests.

FIG. 42 shows repeated pressing of the BFC.

FIG. 43 shows energy harvesting from low-intensity desk work.

FIG. 44 shows energy harvesting from no activity during overnight sleeping.

FIG. 45 shows power harvested from the BFC that is pressed by finger with different sweat generation time.

FIG. 46 shows power of the BFC pressed with different frequencies.

FIG. 47 shows OCV of the PZT chips pressed with different pressure at the center.

FIG. 48 shows the energy harvesting using the PZT chip with different operation conditions.

FIG. 49 shows charging the capacitor using the integrated device with subjects with different sweat rates.

FIG. 50 shows a system flow chart of the integrated system and corresponding voltage values.

FIGS. 51A and 51B show schematics of example embodiments of integrated circuit board for a voltage regulator circuit.

FIG. 52 shows microcontroller unit (MCU) power consumption at different operation voltages.

FIG. 53 shows a capacitor charge flow to MCU.

FIG. 54 shows MCU output voltage and charge to electrochromic display (ECD).

FIG. 55 shows an example of layer-by-layer printing and assembly of the ECD panel.

FIG. 56 shows photographic images of the printed ECD displaying different contents.

FIG. 57 shows current and charge consumption of the printed ECD.

FIG. 58 shows an example of layer-by-layer printing and drop-casting of the sensors.

FIG. 59 shows vitamin C sensor calibration.

FIG. 60 shows an optimization of the vitamin C sensor.

FIG. 61 shows vitamin C determination in sweat from fingertip for 2 subjects.

FIG. 62 shows pharmacokinetic correlation of response to L-Dopa using natural sweat and capillary blood samples.

FIG. 63 shows an example method of determining a concentration of an analyte in at least one of blood, sweat, or interstitial fluid (ISF) of an individual based on some embodiments of the disclosed technology.

FIG. 64 shows an example method of determining a concentration of an analyte in at least one of blood, sweat, or interstitial fluid (ISF) of an individual based on some embodiments of the disclosed technology.

FIG. 65 shows an example method of determining a concentration of an analyte in blood of an individual based on some embodiments of the disclosed technology.

FIG. 66 shows an example method of generating power using a sweat analyte based on some embodiments of the disclosed technology.

FIG. 67 shows an example method of determining a concentration of a biofluid analyte of an individual based on some embodiments of the disclosed technology.

FIG. 68 shows an example device for collecting sweat for the estimation of a concentration of a blood analyte or the utilization of the redox reaction of the analyte for energy generation based on some embodiments of the disclosed technology.

### DETAILED DESCRIPTION

With the exponential increase in the number of patients with diabetes, self-monitoring of blood glucose (SMBG) based on finger-stick blood samples has been a critical component of the management of diabetes. However, self-monitoring or self-testing of blood glucose is limited by the number of permitted monitoring or tests per day. In addition, the inconvenience and pain associated with the standard finger-stick blood sampling deters patients from frequent testing. Accordingly, extensive efforts have been devoted to replacing these blood fingerstick measurements towards improving glucose management protocols.

Continuous blood glucose monitoring has been successfully achieved by using intra-skin needles. However, a completely non-invasive, simple, and reliable approach for glucose detection is yet to be developed and validated. Electrochemical biosensors for monitoring glucose in non-invasive biofluids, such as saliva, tears, sweat, or interstitial fluid, as potential alternative to blood, have thus received a considerable recent attention. Saliva is a readily available biofluid rich in several biomarkers, but its complexity, including the high viscosity and possible food and bacteria contamination, poses major challenges for reproducible glucose analysis. While tears are mostly composed of water with low levels of external contaminants and good glucose tears/blood correlation has been demonstrated, the inherent difficulty in collecting tears has hindered the development of user-friendly glucose tear sensor. The interstitial fluid (ISF) is currently the mostly acceptable biofluid for glucose detection, due to the dynamic equilibrium of such fluid with the blood stream which elevates its diagnostic relevance. Yet, it is not readily sampled and requires microneedles or reverse iontophoretic devices which are subject to biofouling and skin irritation issues, respectively. Finally, sweat analysis has attracted considerable recent attention among these biofluids as an attractive diagnostic biofluid owing to its favorable chemical characteristics and non-invasive nature. Therefore, the majority of the non-invasive electrochemical biosensors has relied on sweat analysis.

Considerable efforts have thus been devoted to the use of the sweat biofluid toward non-invasive glucose monitoring. However, efforts to develop sweat-based rapid and user-friendly glucose self-testing have been largely hindered by the inherent inaccessibility of natural sweat and by mixed reports regarding the correlation between the sweat and blood glucose concentrations. Sweat sampling is commonly carried out by sweat stimulation protocols based on rigorous exercise, iontophoresis or heat. Simpler and faster approaches for accessing this biofluid and improved understanding of the partitioning of glucose molecules from blood to sweat are urgently needed for routine sweat-based user-friendly glucose self-testing.

Such limited understanding leads to mixed reports regarding sweat and blood glucose correlations, including discussions on sweat collection methodologies from different body locations. Several studies demonstrated good sweat/blood correlation in connection to sweat stimulation by agonistic agents or sweat induced by physical activity. However, such correlation is achieved only by performing concurrent blood calibrations for each test analysis, when a calibration curve is attempted to be used to convert the sweat signals, the correlation is lost. The current approach for verifying correlation involves building a calibration curve using standard glucose concentration in either artificial or real sweat matrix. The correlation can be further pursued by employing additional sensors that monitor and correct for possible fluctuations in sweat pH, temperature and salt concentration. The results are usually not satisfactory, especially when comparing the readings from different subjects. The mixed reports on the reliability of sweat-based glucose assays reflect personal variations among individuals, including the sweat rate and skin phenotype properties, related to age, gender or race. Despite extensive research efforts, researchers still do not understand such large variability in the sweat gland function and of skin physiology and structure originated from different populations. For sweat to be properly used as an attractive alternative for blood, these personal variations must be taken into account.

Disclosed herein are methods, materials and devices that pertain to a new rapid and reliable approach to measurement of biomarker concentrations that combines a simple touch-based fingertip sweat sensor (e.g., an electrochemical one) with a new computer-implemented algorithm that addresses personal variations toward accurate estimate of blood glucose concentrations. The new painless and simple glucose self-testing protocol leverages the fast sweating rate on a fingertip for rapid assays of natural perspiration, without any sweat stimulation, along with the personalized sweat-response-to-blood-concentration translation. A reliable estimate of the blood glucose sensing concentrations can thus be realized through a simple one-time personal pre-calibration. Such system training leads to a substantially improved accuracy with Pearson correlation coefficient (Pr) higher than 0.95, along with overall mean absolute relative difference (MARD) of 7.79%, with 100% of paired points residing in the A+B region of the Clarke error grid (CEG). The speed and simplicity of the touch-based blood-free fingertip sweat assay, and the elimination of periodic blood calibrations, should lead to frequent self-testing of glucose and enhanced patient compliance towards improved management of diabetes. Technology disclosed in this patent document also provides a reliable non-invasive option for the frequent monitoring of analytes beyond glucose, e.g., levodopa, ketones bodies, lactate, alcohol, illicit drugs, tetrahydrocannabinol (THC), and cortisol, among others.

The disclosed technology can be implemented in some embodiments to provide, among other features and benefits, a number of significant improvements over the existing technologies used for determining blood concentrations of biomarkers based on sweat analyte responses of the biomarkers. In particular, the disclosed technology can be implemented in some embodiments to address inter-individual variability for accurate translation of sweat analyte responses of biomarkers to values of concentrations of these biomarkers in blood. Such new personalized data processing methods provided by the disclosed technology are combined with a touch-based fingertip sweat analysis. In some embodiments of the disclosed technology, sweat is collected upon skin contact with a collecting hydrogel, then diffuses through the gel to a sensor where analytes present in the sweat are measured.

In some embodiments of the disclosed technology, a personalized data processing method includes determining concentration of an analyte in sweat (e.g., a sweat sample) using a sensor. For example, a glucose oxidase-based biosensor can be used for measuring glucose concentration in the sweat and a molecular imprinted polymer (MIP) based sensor device can be used for measuring cortisol concentration in the sweat. In some implementations, the sensor can include a sweat collection device, which can include a sweat collecting layer comprising, for example, a hydrogel such as, e.g., polyvinyl alcohol (PVA), agarose or glycerol. The sweat collecting layer can be positioned adjacent to or laid on top of a biosensor built using screen-printing, sputtering, inkjet or any other appropriate sensor fabrication technique. Passive sweat can be collected from the skin upon direct contact with the sweat collecting layer. After contacting the skin for a determined amount of time, the collected sweat diffuses through the hydrogel layer, reaching the recognition element or layer of the sensor, where the analyte concentration is measured. Several sensing techniques can be used for the analyte concentration measurements including but not limited to electrochemical, affinity, and optical based ones.

In some embodiments of the disclosed technology, the personalized data processing method can further include determining a personalized (i.e., for a given individual) correlation equation using the determined concentration of the analyte (e.g., glucose) in sweat. For this purpose, analyte concentration measurements are performed, e.g., periodically, over the course of, e.g., several days using the sensor and validated using appropriate approaches. For example, the concentration of glucose in sweat determined using the sensor (which is related to the sensor's output signal intensity, for example) can be validated using a commercial blood glucometer. For example, blood sample can be collected and analyzed prior to (or concurrently with or (immediately) after) each corresponding measurement of the glucose in sweat using the sensor built based on some embodiments of the disclosed technology. A measurement of glucose concentration in sweat performed using the sensor and the corresponding measurement of glucose concentration in blood performed by, e.g., using the commercial blood glucometer provide a data point for the dependence of the glucose concentration in blood, as measured by the commercial blood glucometer, vs. the glucose concentration or level in sweat, as measured using the sensor. A linear slope and intercept of the dependence are obtained for each day of measurements using data points collected during the day. After data collection over the several day period, the values of the linear slopes and intercepts are averaged, and a personalized universal equation is derived for direct conversion of the sweat sensor signal intensity to the blood glucose concentration.

The disclosed technology can be implemented in some embodiments to provide a new approach to sweat-to-blood signal translation, e.g., a new methodology to translate sweat biomarker measurements to reliable estimates of blood concentrations of the biomarkers based on personalized data processing accounting for inter-individual variability. Current sweat sensors rely on extensive exercising, heat or chemical stimulation for sampling sweat, thus demanding time, energy and power consumption. In some embodiments of the disclosed technology, the personalized data processing method can include processing of the signal obtained using collection of passive natural sweat without the need of performing a physical exercise or any additional sweat stimulation steps or activity. The disclosed technology can be implemented in some embodiments to ensure that personal differences in sweat rate or skin properties between individuals are accounted for. Some sweat-to-blood translation methods can produce conflicting results related to correlation of concentration of analytes (e.g., glucose, cortisol, lactate, etc.) in sweat and concentration of those analytes in blood. The discrepancies in the results are mostly related to the sweat collection and data processing steps. However, the disclosed technology can be implemented in some embodiments to provide a new and precise methodology for sweat analysis including the sweat collection, sensing, and data processing steps.

The disclosed technology can be implemented in some embodiments to provide a reliable non-invasive option for the frequent monitoring of analytes such as glucose, levodopa, ketones bodies, lactate, alcohol, illicit drugs, tetrahydrocannabinol (THC), and cortisol. The existing commercial glucose meter (glucometer) requires a finger prick blood testing protocol which is invasive, inconvenient and painful for repeated frequent testing. The touch-based glucose test implemented based on some embodiments of the disclosed technology allows such frequent glucose measurements and obviates the need for periodic blood-based measurements and validations. The simplicity and speed of the touch-based blood-free fingertip assay according to the disclosed technology holds considerable potential for reliable frequent self-testing of glucose towards improved management of diabetes. Also, there is no commercially available test for cortisol detection at the moment. Methods according to the technology disclosed herein can easily translate the levels of glucose and cortisol detected in sweat to blood glucose and cortisol concentration values and require simply touching a sensor with a fingertip and do not need any invasive and sweat-inducing protocols.

In some embodiments of the disclosed technology, data is acquired using a sweat touch-based sensor, for example, daily for a period of, e.g., a week and validated using appropriate approaches. For example, determination of sweat glucose concentration provided by the sensor can be validated using a commercial blood glucometer and cortisol levels can be validated using affinity tests (e.g., using immunosensors). The initial data collection is used for estimating the personal slope and intercept of the dependence that relates the analyte concentration, as measured by the sweat sensor, and the analyte concentration, as measured by a reference device (e.g., a commercial blood glucometer), and these personalized factors or parameters can be used over several weeks without the need for parallel blood testing. A personalized universal equation is thus used for direct conversion of the sweat analyte signal intensity to the blood analyte concentration.

The data collection and processing based on some embodiments of the disclosed technology can be performed by measuring glucose levels in sweat collected from a fingertip. In some implementations, the working electrode of a screen printed 3-electrode electrochemical sensor system is modified with the enzyme glucose oxidase and a polyvinyl alcohol (PVA) hydrogel can be placed over the modified sensor to serve as the sweat collector layer. Sweat is collected from the fingertip during, e.g., 1-minute touching after proper washing of the hands. After collection, sweat glucose signal is obtained by chronoamperometry. The signal is obtained twice a day for one week and validated against a commercial blood glucometer. A linear correlation between the two points (sweat and blood glucose) is obtained for each day of analysis and an averaged slope and intercept of the dependence is calculated for the user. These personalized values account for the individual sweat parameters such as sweat rate and composition. In some embodiments of the disclosed technology, a personalized general equation is generated based on the personalized values and then is used to directly translate the sensor signal into blood glucose concentration values. The disclosed technology can be implemented in some embodiments to use other analytes different from the fingertip sweat, such as levodopa, lactate, alcohol, illicit drugs, tetrahydrocannabinol (THC), and ketones bodies, for example, by simply modifying the electrode surface that suffices to the analyte.

In some embodiments of the disclosed technology, sweat cortisol levels can also be measured by touching the PVA gel with fingertip, e.g., for 30 sec after 2 min of washing hands. The cortisol sensor includes a molecular imprinted polymer (MIP) layer containing a signal indicator and cavity for cortisol detection, providing a label-free MIP sensor, which does not need an additional external signal indicator for the measurement with high selectivity. The signal indicator can be any material that has redox characteristics such as, for example, Prussian blue, ferrocene, methylene blue, or others. A current response using chronoamperometry is measured after 2 min of incubation time to have the binding process between the MIP layer and cortisol. To validate the sensor performance, competitive immunosensor for cortisol is introduced using iontophoresis-induced sweat.

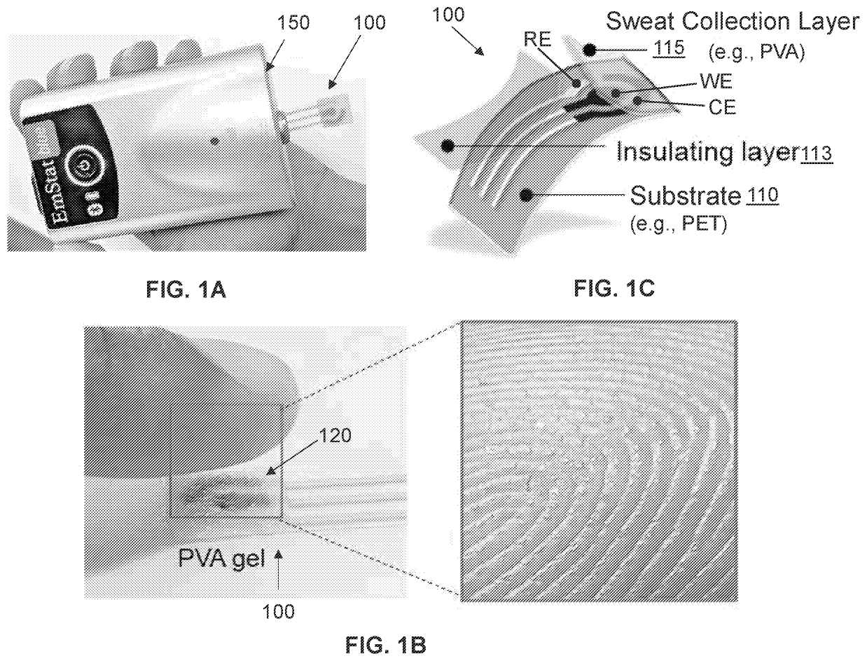

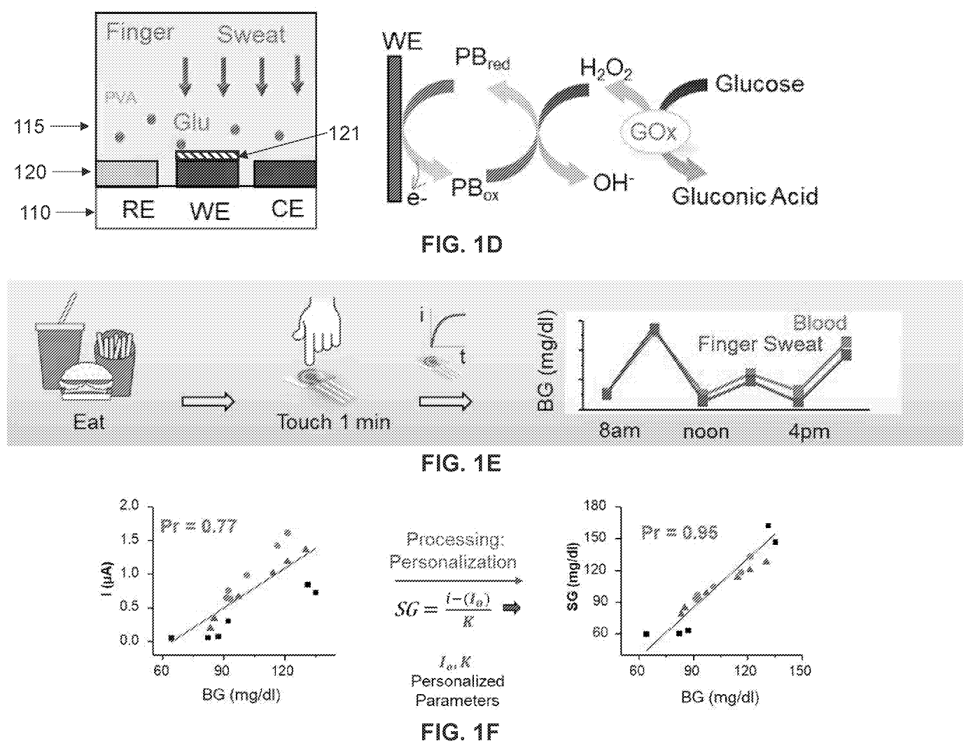

FIGS. 1A-IF show an example of a bloodless fingerstick sweat analyte sensor 100 based on some embodiments of the disclosed technology. Specifically, FIG. 1A shows a portable sensor data processing device 150 (e.g., such as a hand-held potentiostat) coupled with the bloodless fingerstick sweat analyte sensor 100 (also referred to as a “touch sweat sensor”) in accordance with some embodiments of the disclosed technology for electrochemical determination of analyte in sweat. In this example, the target analyte is glucose, and chronoamperometry is used with fixed applied potential (e.g., of −0.2V) to perform electrochemical detection of a target blood biomarker present in sweat deposited over the electrodes of the sensor 100.

FIG. 1B shows an image of an example implementation of the touch sensor device 100, showing a user's fingertip making contact with an electrode assembly 120 of electrochemical sensing electrodes of the sensor 100, demonstrating the sweat glands, sweat collection protocol, and sweat collection layer (e.g., polyvinyl alcohol (PVA) layer) of the sensor 100.

FIG. 1C shows a diagram illustrating an example embodiment of the touch sweat sensor 100, which includes a substrate 110 (e.g., comprising PET); an example embodiment of an electrode assembly 120 embodied as a three-electrode contingent (e.g., a working electrode (WE), a counter electrode (CE), and a reference electrode (RE)), e.g., which can be formed as a screen printed sensor; an insulating layer 113 disposed over electrical interconnects 117 to couple the electrode assembly 120 to an interface region (e.g., contact pads) of the sensor 100; and a sweat permeation layer (also referred to as sweat collection layer) 115, which in some example embodiments includes one or more PVA layers.

FIG. 1D shows an example implementation of sweat collection and target blood biomarker detection, e.g., of the biomarker glucose, from a subject's fingertip, through the example PVA gel (i.e., an embodiment of the sweat collection layer 115), which reaches the electrode assembly 120 for electrochemical detection. In some embodiments, for example, at least one of the electrodes, (e.g., the working electrode (WE)) includes a chemical recognition layer 121 that includes one or more chemical reaction facilitators to catalyze or otherwise facilitate a chemical reaction involving the target biomarker that causes generation of an electrical signal that is detectable by the electrode assembly 120. In the example shown in FIG. 1D (right side panel), the chemical recognition layer 121 includes Prussian blue (PB) layer modified with the enzyme glucose oxidase (GOx) that is formed on an example screen-printed working electrode to provide an electrochemical sensor transducer for selective detection of the hydrogen peroxide product (H₂O₂) of the glucose/GOx enzymatic reaction, which generates an electrical signal at the working electrode, the electrode assembly 120 can detect, indicative of a parameter (e.g., concentration) of the glucose in the sweat fluid. For example, as illustrated in the schematic of FIG. 1D, i.e., depicting the enzymatic reaction in the GOx working electrode, glucose is converted into gluconic acid and hydrogen peroxide. The hydrogen peroxide molecules are then detected by the PB modified working electrode. The electrical signal detectable by the electrode assembly 120 is processed by a data processing device, such as the portable sensor data processing device 150 shown in FIG. 1A, to determine a parameter of the glucose.

FIG. 1E shows a workflow for sweat glucose detection using the touch-based sweat sensor. After 20 minutes of food intake sweat is collected upon touching the sensor for 1 minute; amperometric detection is immediately performed measuring the sweat glucose. Upon using the personalized transduction equation, shown in FIG. 1F, the sweat signal is converted to a blood glucose level.

FIG. 1F shows data processing for sweat and blood correlation. Current signal collected from three subjects is directly correlated with blood values, showing a Pearson's r (Pr) value of 0.77. The sweat-based blood glucose concentration (SG) is thus estimated using the personalized parameters K and Io. After applying the personal equations to each set of data, the sweat-blood correlation increases to 0.95.

As mentioned above, the disclosed technology can be implemented in some embodiments to include a combination of a new personal algorithm for correlating sweat and blood concentrations of a target analyte (e.g., glucose) with the simple and effective touch-based fingertip sweat collection and electrochemical detection towards rapid, reliable and user-friendly self-testing of glucose (FIGS. 1A and 1B). The fingertip has high density of sweat glands (˜400 glands cm⁻²), producing sweat in relatively high rates over the range of 50-500 nL cm⁻² min⁻¹. Such natural fingertip perspiration has been used recently for optical detection of illicit drugs, electrochemical detection of sweat lactate and caffeine and LC-MS/MS measurements of tryptophan and dopamine.

Methods and devices based on some embodiments of the disclosed technology can leverage the fast sweat rates on the fingertip for rapid glucose measurements in natural perspiration, without the need for rigorous sweat-inducing exercise activity or iontophoretic sweat stimulation. Collection of sweat from the fingertip is based on touching the surface of a sweat-absorbing polyvinyl alcohol (PVA) porous hydrogel membrane capable of pulling the sweat droplets from the fingertip by capillary pressure, during a controlled time (FIGS. 1A-1D). The porous PVA membrane is placed on the electrochemical biosensor for subsequent glucose detection upon sweat transport towards the enzymatic layer covering the Prussian blue (PB) transducer. The glucose detection is performed via selective reduction of the enzymatically-liberated hydrogen peroxide at the PB transducer (FIGS. 1C-1D). Such fast and simple touch-based blood-free fingertip sweat glucose assay holds considerable promise for improved patient compliance and enhanced diabetes management.

However, while the attractive fingertip natural perspiration could greatly simplify glucose sweat measurements, such direct measurements do not account for variability among individuals and generally display non-satisfactory correlation to blood glucose assays. To address these issues, technology disclosed in this patent document uses a new ‘personalized’ mathematical approach that improves substantially the sweat-blood glucose correlations and the overall accuracy of such diabetes testing. Such simple one-time personal calibration accounts for variations in the sweat rate and skin properties among individuals through a distinct sweat-to-blood translation algorithm following a one-time training of the system. The short personal system training involves blood validated sweat signals to estimate the average individual slope (K) and intercept (Io) for each person, for obtaining a personalized sweat-to-blood translation factor (FIG. 1F). Such initial training and treatment lead to substantially higher Pearson correlation coefficient (Pr) of 0.95, and significantly higher accuracy reflected in an overall mean absolute relative difference (MARD) of 7.79%, with 100% of paired points in the A+B region of the Clarke error grid (CEG). These substantial improvements are realized without the need for additional sensors and complex microfluidic network for correcting and normalizing the results. Following such one-time personal training of the system, accurate glucose blood levels can be estimated directly from the individual sweat glucose response over extended periods of several weeks, based solely on his/her sweat signals without the need of blood sampling (FIG. 1E). A single blood calibration is recommended once or twice a month. Such single periodic measurement is analyzed by the software that screens for outliers and updates the existing personal parameters. When using the new algorithm among multiple subjects, the Pr values increase from 0.77 (raw sweat signal to blood glucose) to more than 0.95 (calculated sweat glucose to blood glucose), as shown in FIG. 1F for 3 subjects. Detailed studies demonstrate also substantially higher accuracy upon using both the personal intercept and slope compared to using the slope alone. Such greatly improved correlation is achieved even though the values of the slopes and intercept values are substantially different between subjects. In some implementations, the slope values correspond to the fingertip sweat rate, while the intercepts reflect multiple factors based on the individual skin properties and sweat composition. Note, however that negligible electroactive interferences are expected at the PB-based electrode transducer using detection potential of −0.20V. This simple mathematical treatment can be readily integrated in a software (in, e.g., a hand-held meter or a smartphone app), providing a built-in personal calibration towards autonomous estimate of the sweat-based blood glucose concentration (SG). Our extensive data strongly support the subject personal equation based on an initial blood validated sweat response. Once such personalized translation is obtained, blood glucose levels can be directly and reliably estimated from sweat measurements without the need for blood fingerstick validation. A single blood calibration is recommended once or twice a month. Such single periodic measurement is analyzed by the software that screens for outliers and updates the existing personal parameters. By accounting for variability among individuals the new approach provides effective normalization of the sweat glucose response, leading to greatly improved inter person sweat-to-blood correlation parameters, with potential application for the monitoring of other sweat biomarkers.

Embodiments of the sweat permeation layer 115 include a hydrogel that can be made from an aqueous precursor that contains a solution of monomers or polymers that can be later chemically or physically crosslinked and solidified. The precursor can optionally contain a template material that can be removed from the solidified hydrogel to create pores within the gel structure. The creation of these porous structures within the hydrogel can aid the material transfer of the analyte from the skin surfaces to the electrode surfaces. The size of the pores can be adjusted by varying the type, amount, and removal method of the template materials, and is in general macroporous, with the size of 50 nm or greater, including a pore size in a range between 1 m to 1 mm, where the pores can be configured to be substantially the same or similar size regime, or a varying size regime. In some implementations, for example, when the aforementioned crosslinking takes place on top of the electrode in-situ, the gel may provide a better bonding to the electrode surface. The gel-on-electrode combination is made for convenient disposable uses. One such example includes a porous PVA hydrogel.

Example materials used in implementations to produce and test an example embodiment of a bloodless fingerstick sweat analyte sensor 100, including an example PVA hydrogel for a sweat permeation layer 115 of the sensor 100. Polyvinyl alcohol (PVA) (MW ˜89,000), phosphate buffer solution (PBS) (1M, pH=7.4), potassium hydroxide (KOH), sucrose, sodium chloride, potassium chloride, glutaraldehyde, glucose oxidase (GOx), glucose, silver/silver chloride ink and Prussian Blue (PB) carbon ink, dielectric ink, and Ecoflex 00-30 were used to evaluate the methods and devices implemented based on some embodiments of the disclosed technology. Chronoamperometric measurements can be performed using a potentiostat.

The electrodes for the finger-based glucose sensor are fabricated by screen-printing using a semi-automatic MMP-SPM printer and custom stainless steel stencils developed, with dimensions of 12 in×12 in and 75 μm thickness. The electrodes are printed layer-by-layer. Firstly, the silver/silver chloride ink is printed onto a polyethylene terephthalate (PET) substrate as the interconnection and reference electrode, followed by printing a layer of PB carbon ink as the working and counter electrodes. Each layer is cured at 80° C. for 10 min in the oven. The working electrode is modified with 2 μL of a GOx 40 mg/ml in 0.1 MPBS pH 7 containing 10 mg/ml BSA. After drying at room temperature, 0.5 μL of a 0.5% solution of glutaraldehyde in water is added to the GOx modified working electrode and left to dry overnight at 4° C.

For fabrication of the porous PVA hydrogel, the stock solutions of the PVA (MW ˜89,000) and KOH, dissolved in water, are prepared by 1:10 and 1:5 weight ratio, respectively. Then, 10 g of PVA solution is transferred to the beaker followed by dropwise adding 14 g of KOH solution and 2 ml of water containing 2.6 g of table sugar under mild stirring condition to form a hydrogel precursor. 15 g of the precursor is then poured into a Petri dish (diameter ˜9 cm) and left in a vacuum desiccator to remove excess water and allow cross-linking, until only ⅔ of the weight of the precursor is left. The crosslinked PVA gel is then soaked in 0.1 M PBS buffer to remove the sugar template and the excess KOH, until the gel reached a neutral pH. The gel (1 mm thick when soaked) can then be cut into desired sizes (1×1 cm²) and stored in PBS for subsequentuse.

In some implementations, an on-body evaluation on human subjects can be conducted as follows. The glucose response is recorded by measuring the current difference, between the background signal (PVA gel prior to touching) and the sweat glucose signal at an applied potential −0.2 V (versus Ag/AgCl) for 1 min. Patients are asked to clean their index fingers with wet (DI water). After cleaning, sweat is allowed to accumulate on the fingertip for 3 minutes, followed by touching the PVA sweat collector gel for 1 minute. Right after touching, the sweat glucose signal is recorded.

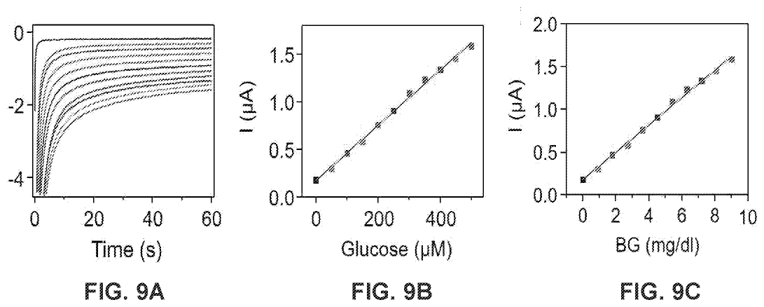

The touch-based non-invasive sweat fingertip glucose detection includes two steps of the sweat collection by touching of a sweat absorbing porous hydrogel membrane (covering an enzymatic biosensor) and the amperometric detection of the product of the biocatalytic reaction using the biosensor (FIG. 1). The high density of sweat glands in the fingertip ensures sufficient biofluid volume for reliable and reproducible glucose measurements. Sweat collection from the fingertip is performed upon direct contact of the fingertip with the sweat permeation layer 115, upon the fingertip touching the sweat permeation layer for a minimal amount of time, e.g., such as for about 1 minute. In some example embodiments, the sweat collection layer 115 includes a porous polyvinyl alcohol (PVA) hydrogel material placed over the sensor surface to facilitate the collection and transfer (i.e., permeation) of sweat fluid containing its constituents, including the target analyte, across the opposing sides of the layer. In some implementations, for example, the PVA hydrogel includes pores having a pore size of greater than 50 nm, which can include up to 1 m or up to 1 mm. From the direct contact of the fingertip with the sweat permeation layer 115, tiny volumes of sweat fluid are taken (collected) in and transfer through the layer 115, where the collected sweat diffuses to the recognition layer (i.e., the modified and/or unmodified electrodes of the electrochemical sensor) where an enzymatic reaction occurs for detecting a parameter about the analyte in the sweat, which can be processed to determine a parameter of the analyte in blood (discussed later in this disclosure). A flexible polyethylene terephthalate (PET) is used as a substrate to screen print the three-electrode (120) system electrochemical sensor (FIG. 1C). The sensor 100 is designed to fit a handheld potentiostat for decentralized analysis (FIG. 1A). As shown in FIG. 1D, the sensor 100 includes a substrate 110, electrodes (e.g., working electrode WE, counter electrode CE, reference electrode RE) 120, and a porous sweat permeation layer 115, such as the example PVA layer described above. The screen-printed Prussian blue working electrode transducer is modified with the enzyme glucose oxidase (GOx) and used for selective detection of the hydrogen peroxide product of the glucose/GOx, enzymatic reaction (FIG. 1D) with a sensitivity of 2.89 nA·μM⁻¹ as shows in FIGS. 9A-9C.

Such painless touch-based glucose sensor represents a promising non-invasive approach to improve diabetes monitoring by increasing the frequency of glucose testing. However, analyzing glucose from sweat is a challenging task. Sweat glucose levels can fluctuate depending on the methodology used for sweat collection. For example, sweat obtained during exercising can underestimate the glucose levels, while iontophoresis can overestimate the glucose levels due to accumulation of glucose on the iontophoretic gels. In addition, contamination from skin components, such as bacteria, body creams and even glucose itself can also influence in the measured glucose values. The glucose concertation in sweat ranges from 0.01-1.11 mM, are significantly lower than the blood concentrations (2-40 mM). Thus, the fingertip touch glucose sensors ensure user-friendly sweat collection as it does not involve exercising or chemical stimulation of the sweat glands. The electrochemical signal is then converted into blood glucose levels using the new personalized algorithm to account for the individual skin properties and sweat rate. The Pr values for different subjects increase from 0.77 to more than 0.95 when using such personalized approach (FIG. 1F).

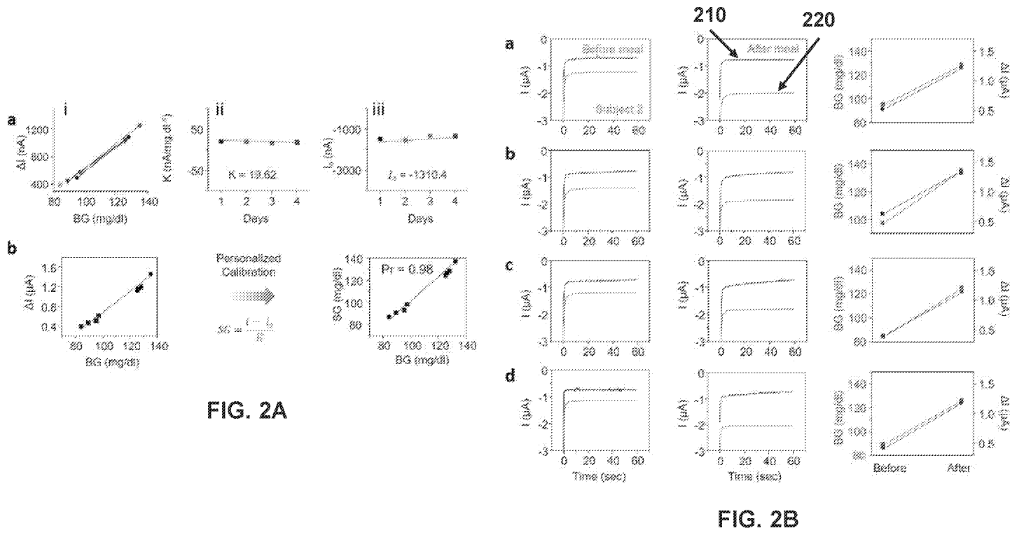

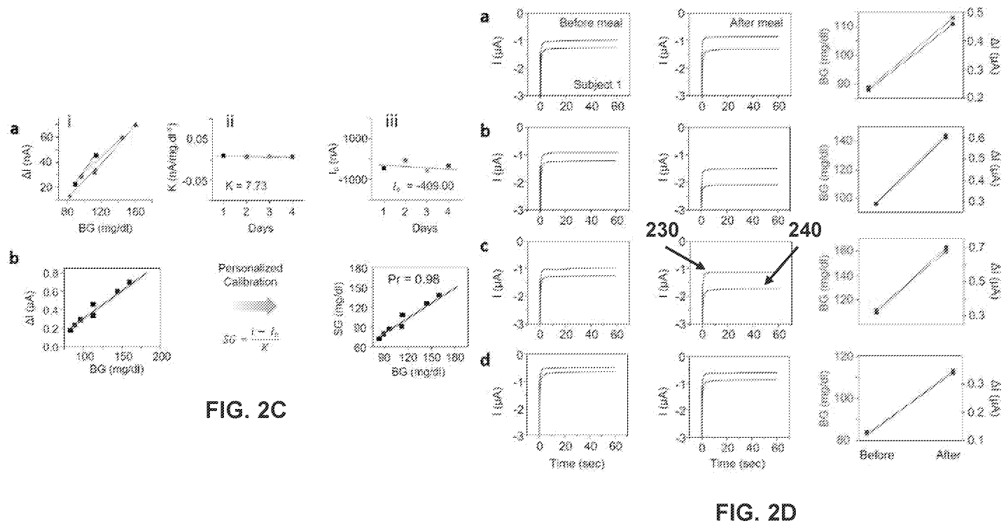

FIGS. 2A-2D show an example of data processing protocol for personalized transduction equation. Specifically, FIGS. 2A and 2C show individual values of the signal from sweat for each day are correlated with the blood values generating a linear plot with specific slope and intercept values (i-iii). The slopes and intercepts obtained for each day are then averaged and a personalized equation is generated for each user (b). Upon obtaining such personal transduction equation, the current signal from the glucose in sweat is used for the direct translation of the signal to blood glucose values. FIGS. 2B and 2D show data from sweat glucose monitored for two subjects for four days, twice a day. Top curves (e.g., 210, 230) correspond to the signal prior the sweat collection (only PVA gel) while bottom curves (e.g., 220, 240) correspond to the sweat glucose response. Prior to each analysis, a commercial blood glucose meter is used to measure the blood glucose values of the user

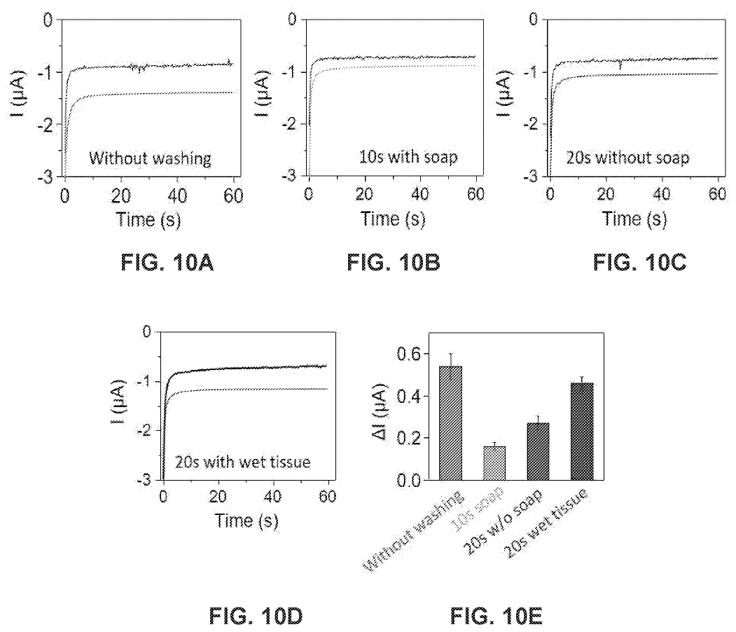

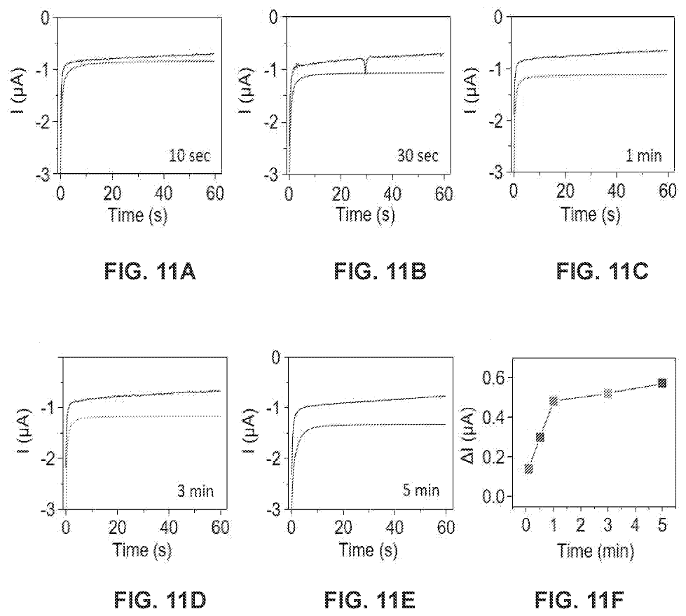

Following the successful implementation of the touch-based sweat-collection/electrochemical detection protocol sensor, the disclosed technology can be implemented in some embodiments to provide a new mathematical approach for correlating sweat glucose response to the blood glucose concentrations. Such personalized sweat-to-blood translation algorithm includes measuring the fingertip sweat glucose response and calibrating these current values using the blood glucose levels with a commercial glucometer. Measurements are performed daily at the same time (FIGS. 2 B, D). Sweat and blood glucose levels are measured before and 20 minutes after consuming a meal. An optimized protocol for the finger sweat analysis is strictly followed. First, patients are asked to clean their index finger using a wet tissue and wait for 3 minutes; next, they are asked to touch the sensor for 1 minute. Subsequently, the sweat signal is measured using chronoamperometry at a fixed potential of −0.2V for 60 seconds. It is noticed that the use of soap for cleaning the finger decreased the measured signal, due to potential interaction of surfactant residues with either the PVA gel or enzyme layer. Therefore, a mechanical cleaning with water is used FIGS. 10A-10E. This cleaning protocol is followed by an optimal touching time of 1 minute (FIGS. 11A-11F). The calibration plot for each day is analyzed and the average slopes and intercepts are calculated (FIGS. 2 A and 2C (i-iii)). The following equations are used to translate the sweat glucose response to blood glucose concentrations:

$$K = \frac{Δ {i}_{SG}}{Δ BG} \qquad ( Eq . 1 )$$

$${I}_{o} = {i}_{SG} - ( K \times BG ) \qquad ( Eq . 2 )$$

$$SG = \frac{{i}_{SG} - ( {I}_{o} )}{K} \qquad ( Eq . 3 )$$

The slope (K) and intercept (Iₒ) have been calculated using Equations 1 and 2, respectively. As shown in FIGS. 2A and 2C, the slope corresponds to the variations in the current obtained from the glucose sweat sensor (Δi), correlated with the changes in blood glucose concentration (ΔBG) obtained with a glucometer. i_{SG} represents the current response of the sweat sensor and BG is the blood glucose concentration (FIGS. 2A and 2C ii). Finally, as shown in equation 3, the concentration of sweat-based glucose can be estimated using equation 3, from the current response of the glucose sweat sensor, and the average K and Iₒ values (FIGS. 2A and 2C b).

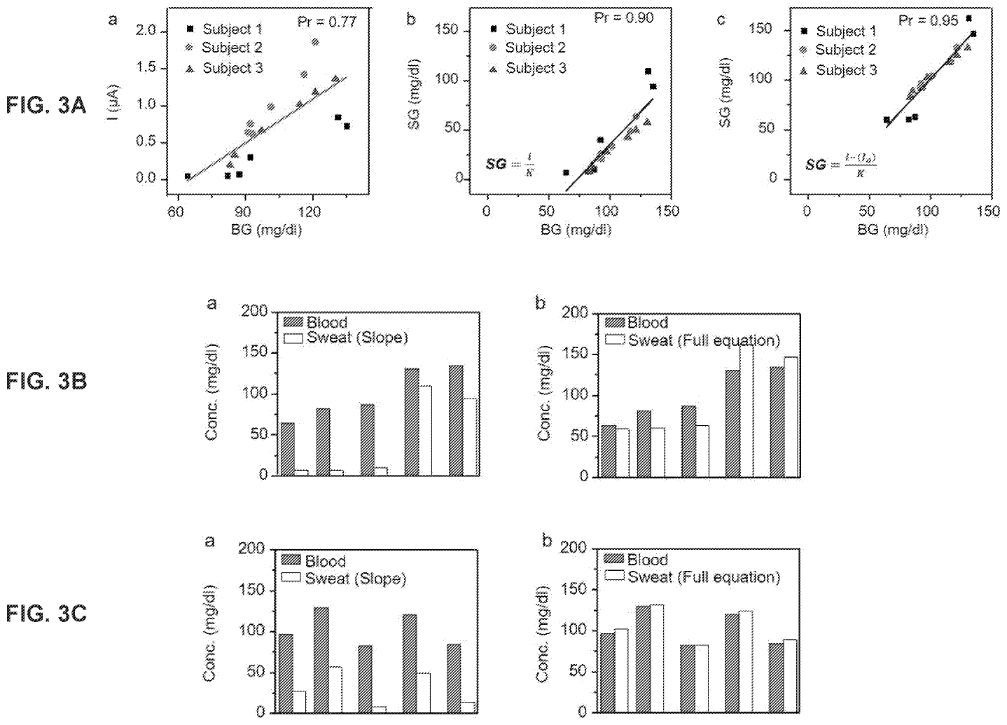

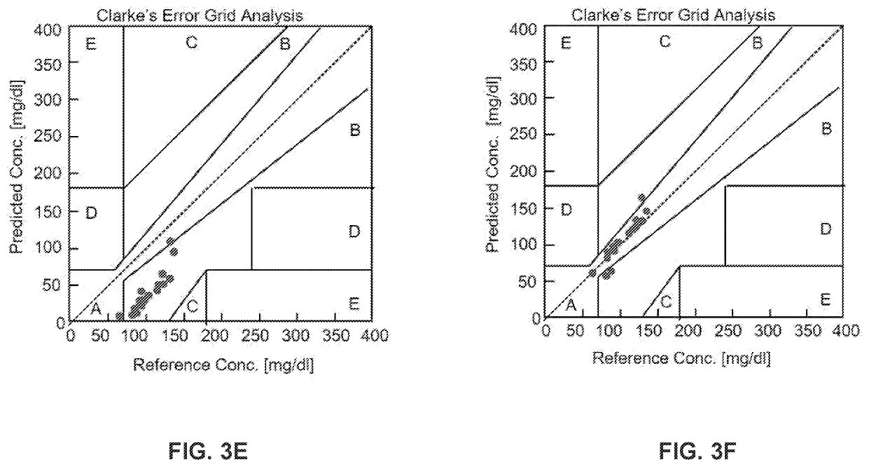

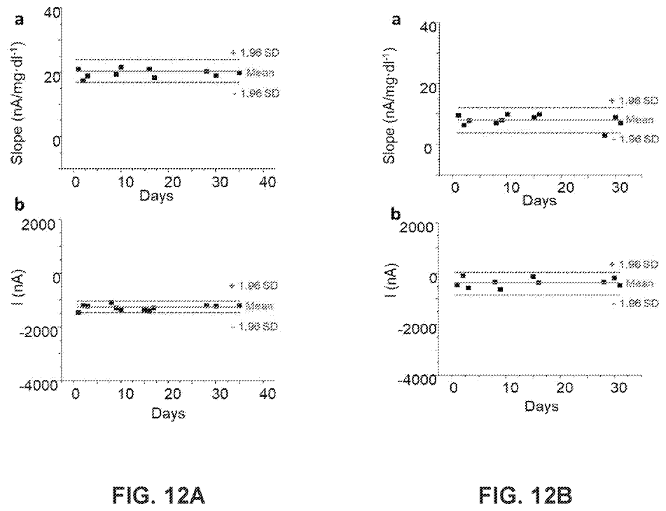

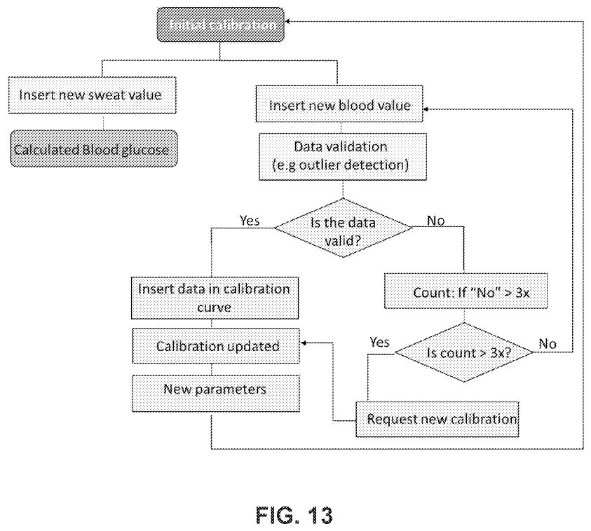

As shown in FIGS. 2A and 2C (b) (iii), the resulting sweat-based estimated blood glucose concentrations correlate closely with the reference fingerstick blood glucose concentrations (Pr=0.98, for both subjects), despite the large differences in the individual slope and intercept values. Note also from FIGS. 2A and 2C (a) (ii and iii), that these individual K and Jo parameters are stable throughout the month (FIGS. 12A-12B). A built-in software is used to assess the stability of these personal parameters, along with a single biweekly or monthly blood calibration point. This safety calibration point is analyzed by the custom software that identifies potential changes and updates the existing personal parameters (FIG. 13). The normalization of the sweat parameters represents the main advantage of this new approach once the matrix (sweat) variables are eliminated, increasing the correlation when data from multiple subjects are analyzed (FIG. 3).

FIGS. 3A-3F show examples of effects of different data transformation steps that translate current response of the sweat glucose sensor to the sweat-based blood glucose concentration. Specifically, FIG. 3A shows data transformation steps from the (a) current response to sweat glucose concentration applying (b) personalized slope only and (c) personalized slope and intercept from different subject (n=18). (FIGS. 3B-3D) Bar plot displaying the correlation of the measured blood glucose level and the sweat-based blood glucose concentration calculated by using (a) only the personalized slope and (b) the slope along the intercept from different subject. (FIGS. 3E-3F) Clarke error grid (CEG) analysis results using the personalized slope (E) alone and using both the personalized slope and intercept (F).

The personalized parameters are obtained for three subjects and applied to a new set of six measurements obtained for each user. The current signals of the sweat sensor are plotted against the reference blood values measured with a glucometer (FIG. 3A (a)). A Pr value of 0.77 is observed for the correlation between the sweat current response and the blood glucose values of the three subjects, indicating limited correlation of the fingertip sweat glucose response with the blood glucose levels. The mathematical personal treatment is subsequently applied to the results shown in FIG. 3A (a). In order to demonstrate the importance of each personal parameter, the personal slope (K) is initially used alone for converting sweat response to blood glucose values (FIG. 3A (b)). Such conversion has been commonly used in the literature for the signal translation. As shown in FIG. 3A (b), the use of the personal slope results in increasing Pearson's correlation from 0.77 to 0.90. This improvement reflects the personalized feature of the protocol associated with the use of the individual K values for each data set. However, as shown in FIGS. 3B-3D (a), such treatment based solely on the personal slope leads to poor accuracy, with the predicted blood values differing largely from the reference blood concentrations. In contrast, greatly improved accuracy is achieved by including also the personalized intercept values Iₒ in the mathematical treatment (along with the slope ones). This brings the sweat-based predicted blood glucose values significantly closer to the reference values (FIGS. 3B-3D (b)) and leads to further improvement of the Pr values to 0.95 (FIG. 3A (c)).

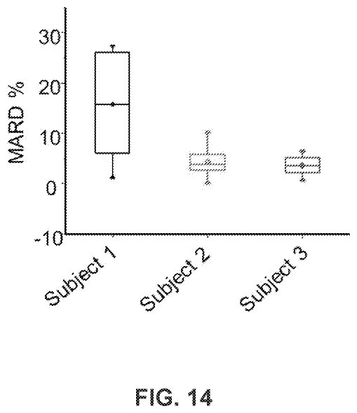

The importance of using personalized full equations can be clearly demonstrated by analyzing and comparing the CEG plot for both situations, involving the use of the slope alone (FIG. 3E) and when the full equation is applied (FIG. 3F). The CEG analysis is commonly used to evaluate the deviation of the clinical significance between the predicted BG concentration and the reference concentration (such as glucometer). Such analysis uses a Cartesian diagram on which the target and predicted BG values are paired. Each pair is in one of five regions of the CEG diagram with zone A indicating the values within 20% of the reference concentration. The pairs located in region A thus represent clinically correct predictions. Therefore, it is highly desirable to have all the results in this zone. Pairs of points within region B are still clinically acceptable (but not for therapeutic decisions), while pairs in regions C, D and E are considered significant clinical errors. The CEG diagram for the touch-based sweat assay using the slope alone shows that the majority (85%) of the points reside in the B section, with only 2 points (12.5%) located in area A (FIG. 3E), corresponding to a fair correlation to reference concentration. In contrast, when using personalization steps involving both the slope and intercept, the CEG analysis reveals that the majority (81.2%) of the points reside in region A while only 3 points positioned in region B (18.8%). Overall, the CEG analysis of FIGS. 3E and 3F demonstrates clearly that personalized calculation—based on both the slope and intercept—affects strongly the correlation of the sweat glucose measurements with the blood reference method towards reliable prediction of the blood glucose concentration. In addition, the measurements shown in FIG. 3A (c) are also used to calculate the mean absolute relative difference (MARD). The aggregate MARD for the touch glucose sensor is 7.79% (ranging from 3.5 to 15.0%. FIG. 14), based on all individual paired data points from the 18 recordings of 3 subjects. Such value (below 10%) reflects the high accuracy of the methodology.

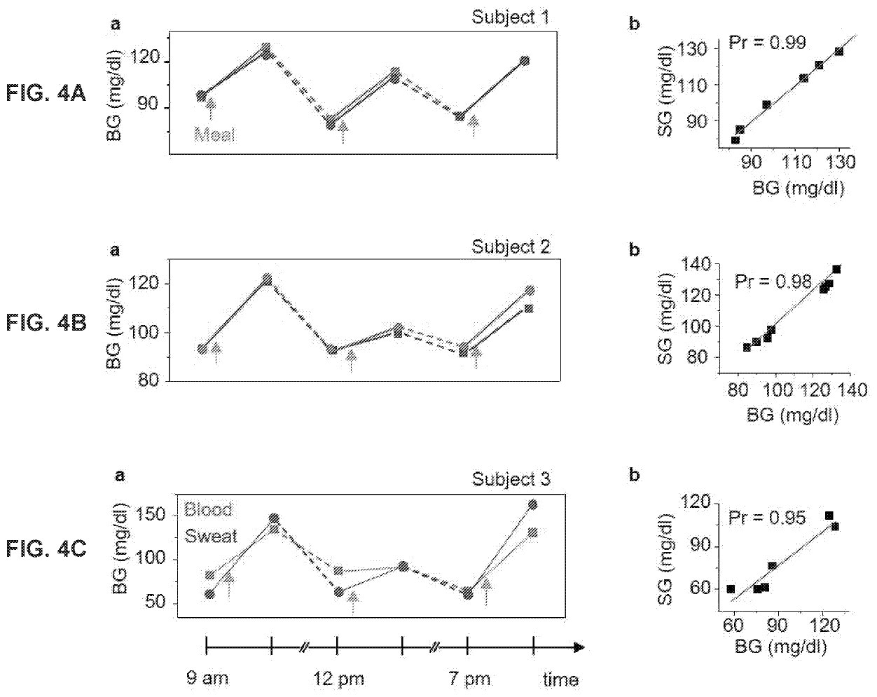

The performance of the touch-based sweat glucose sensor and the corresponding mathematical personalization treatment are evaluated in a “blind” test. The glucose levels from three patients, whose personalized equations are previously established, are monitored during a day long operation, involving 6 measurements obtained before and after the corresponding meal intakes. The same protocol is used for each sweat measurement (cleaning of index finger, waiting 3 minutes, touching 1 minute), along with a new sensor and gel each time. The sweat current signals are translated into predicted blood glucose concentrations using the personal equation of each subject. The calculated blood values from these “blind” tests are shown in FIGS. 4A-C blue (circle) plots. Prior to each sweat measurement, the blood values are measured and saved for comparison. The person responsible for calculating the expected blood concentrations did not have access to the reference blood values. FIGS. 4A-4C (b) show the correlation between the sweat-based calculated blood concentrations and the corresponding blood reference levels. These data show clearly that the dynamics of such sweat-based predicted blood glucose concentration throughout the day is in close agreement with the actual temporal blood glucose profile. Pearson's values are higher than 0.95 (ranging from 0.95 to 0.99) for the three subjects. It is important to notice that these “blind” tests are performed a week after the initial personal system training, reflecting the robustness of method (and the stability of the slope and intercept values). As is demonstrated in FIGS. 12A-12B, the personal equation is stable for at least one month, eliminating the need for intermediate blood fingerpicking. However, a periodic blood calibration (once or twice per month) is recommended to ensure the translation accuracy.

FIGS. 4A-4C show examples of measurement results obtained for whole day sweat glucose measurements (circle markers) along with the corresponding blood measurements (square markers). FIGS. 4A(a), 4B(a), and 4C(a) show glucose levels in sweat collected from the fingertip just before a meal and 20 min after completing the meal using the touch sensor device during the whole day after three meals (shown as arrows). The signal obtained from the sweat sensor is directly translated to blood glucose levels using the personalized translation equation of each user. FIGS. 4A(b), 4B(b), and 4C(b) show the resulting correlation plots and corresponding Pr values.

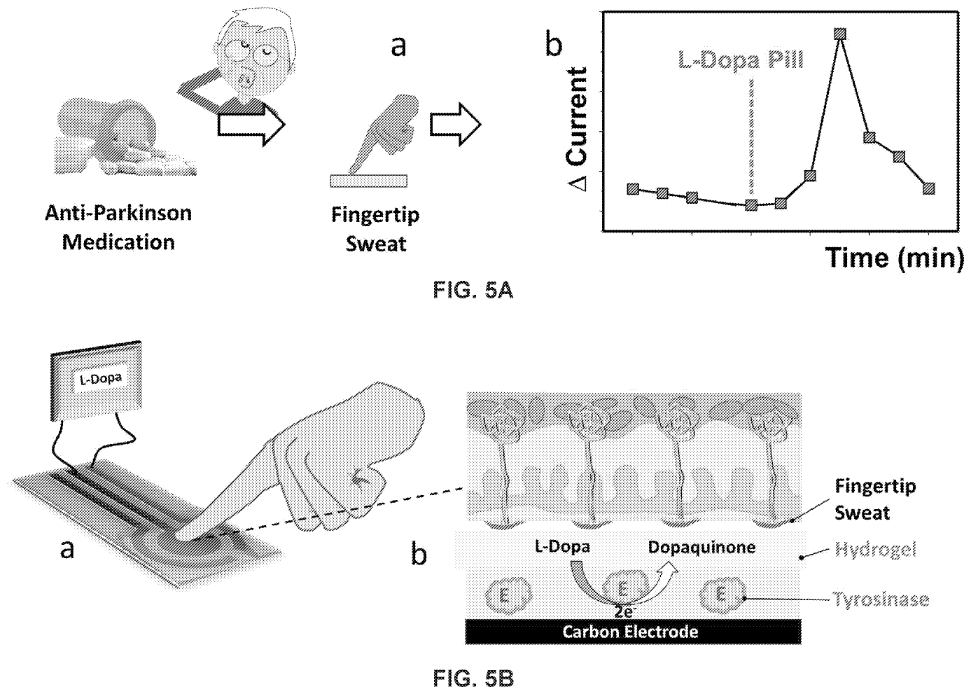

FIGS. 5A-5B show example principles of operation of a fingertip levodopa biosensor based on some embodiments of the disclosed technology. FIG. 5A shows a general procedure followed during monitoring of Levodopa, including (a) 100:25 Levodopa:Carbidopa pill intake is performed followed by the collection of sweat using a fingertip biosensor according to the disclosed technology, and (b) The same measurements procedure is repeated every 10 min over a period of 1 hour. FIG. 5B shows (a) depiction of the finger placed on top of the biosensor, and (b) Zoom image of the finger area shows the sweat secretion from the sweat glands, followed by sweat collection on a hydrogel membrane. The high porosity of the membrane and incubation time allows the diffusion of the sweat into the transducer modified with Tyrosinase. The electrochemical reaction takes place when a negative potential is applied to the biosensor, as a result, 2 electrons are donated from the electrode surface to the analyte collected from sweat, allowing the reduction of L-Dopa to L-Dopaquinone.

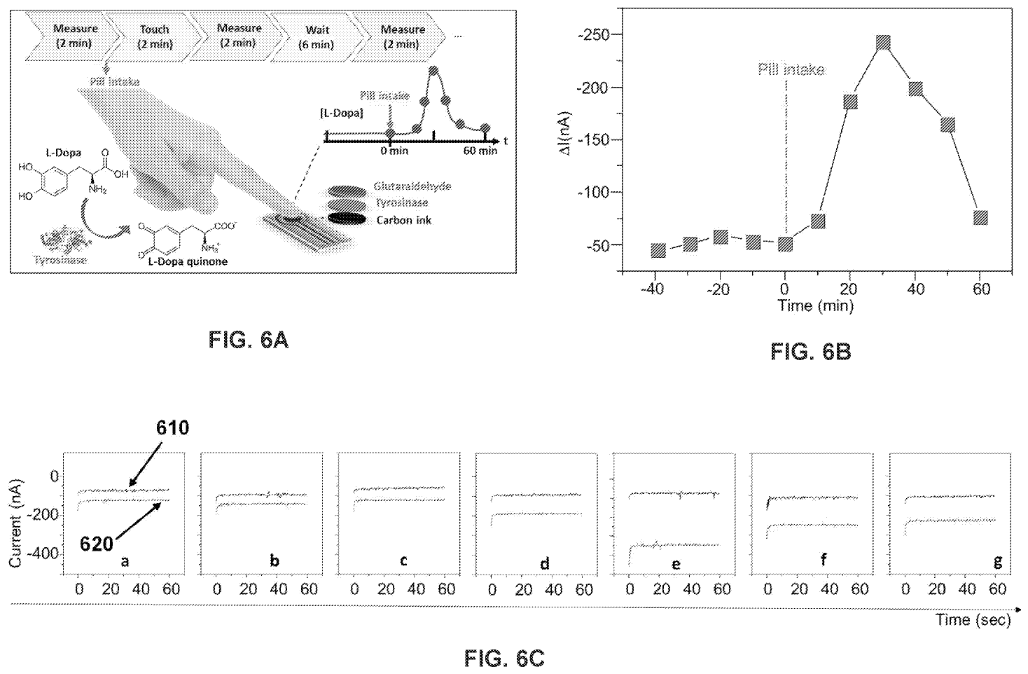

FIGS. 6A-6C show an example timeline followed during Levodopa monitoring in sweat. Specifically, FIG. 6A shows an on-body test starts monitoring the signal output without any sweat. Afterwards, a Levodopa pill is taken orally. After the pill intake, the subject is asked to place the tip of their finger into the hydrogel membrane for about 2 minutes. After the sweat collection step, potential step measurements are performed twice. In order to monitor every 10 min, subjects are asked to wait 6 minutes after this step until completing the 10 minute cycle. Subsequently, subjects repeat the touching and measuring step. The multilayer composition of the biosensor includes a carbon ink electrode modified with Tyrosinase enzyme. The Glutaraldehyde layer allows entrapment of the enzyme, providing stability for the extended operation of the sensor. The negative potential applied to the biosensor allows the reduction of the Levodopa in sweat to L-Dopaquinone. FIG. 6B shows current signals are obtained before and after touching the sensor, this allows the continuous obtention of the current difference (ΔI) every 10 minutes. The example shown displays an example profile obtained by monitoring the ΔI over a period of 1 hour after the pill intake, in which an increase in current is observed after a couple of minutes after the pill intake. FIG. 6C shows chronoamperograms obtained in every 10 minute step show the current obtained before (upper black lines, e.g., line 610) and after (lower lighter-black lines, e.g., line 620) taking the medication.

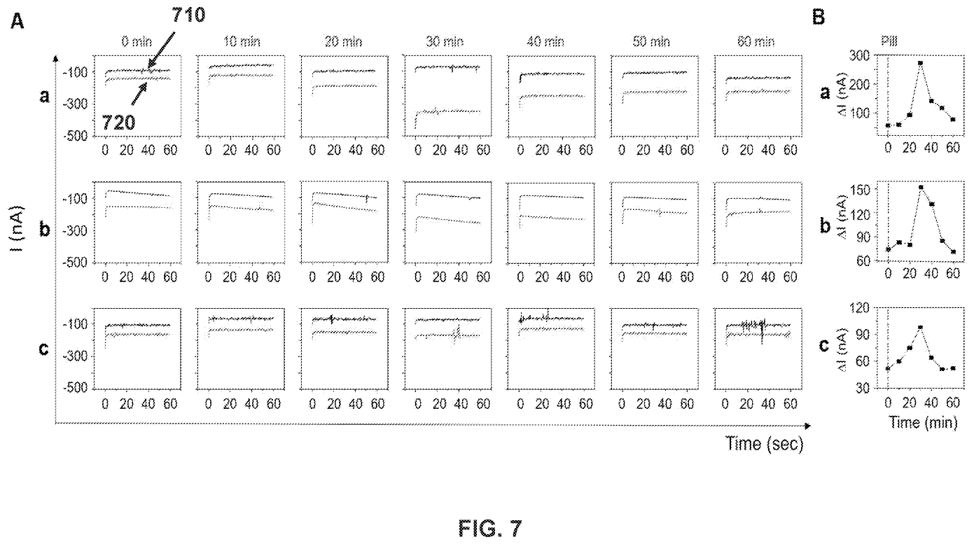

FIG. 7 shows example data collected during an example on-body demonstration of the Levodopa biosensor. The performance of the fingertip sensor is tested on 3 different subjects in 10 minutes intervals over a 1 hour long period of time. The left side of the image displays the choronoamperograms obtained on each time interval. Upper black lines (e.g., line 710) on each set show the current output before any sweat collection. Lower lighter-black lines (e.g., line 720) correspond to the current signal obtained after sweat collection and 100:25 pill intake. The right side of the image shows the current difference (AI) obtained on each 10 minute interval. The dotted vertical line labeled “Pill” sets the time at which the oral intake of the pill is performed.

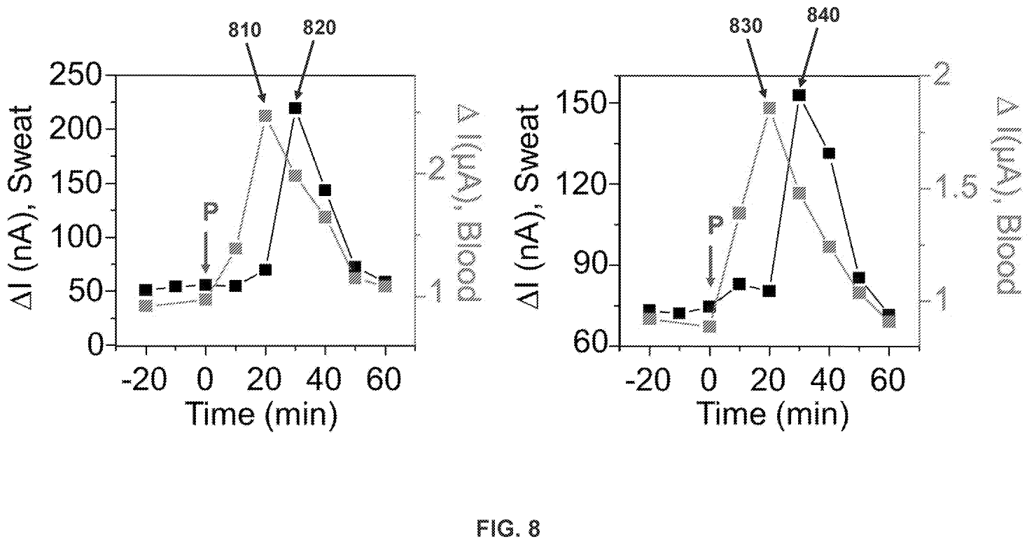

FIG. 8 shows example comparisons of Levodopa profiles obtained via Levodopa measurements in blood and sweat. The current profiles of Levodopa in blood (profiles 810 and 830 in FIG. 8) and sweat (profiles 820 and 840 in FIG. 8) are monitored in 10 minutes intervals for a period time of 1 hour. The arrows “P” mark time instances of the pill intake.

The disclosed technology can be implemented in some embodiments to provide a new non-invasive approach for fast, simple and accurate sweat glucose testing, and a new algorithm for addressing inter-individual variability and obtaining a greatly improved accuracy. Natural sweat from the fingertip is thus used for the electrochemical determination of glucose using a highly selective glucose-oxidase Prussian blue sensor in connection to a sweat collecting hydrogel. The resulting sweat glucose current values are translated into predicted glucose blood concentrations by applying a personalized equation to account for personal variations among the test subjects. In some implementations, using the personal parameters the Pearson (Pr) correlation values increase from a Pr value of 0.77 to 0.95, and lead to a MARD of 7.79% with 100% of paired points in the A+B region of the Clarke error grid. Such greatly improved correlation has been achieved despite of the large variability of the slopes and intercept values among the subjects. The simplicity, speed and accuracy of the new touch-based fingertip assay hold considerable potential for reliable frequent self-testing of glucose towards improved management of diabetes. Such blood-free assay thus represents an attractive non-invasive alternative to fingerstick blood glucose measurements (particularly when frequent glucose measurements are desired). The new personalized data processing approach could be applied for a wide range of electrochemical sweat assays of important analytes such as levodopa, alcohol or cortisol.

In some embodiments of the disclosed technology, a device for collecting sweat for the estimation of a concentration of a blood analyte or utilization of a redox reaction of such analyte for energy generation includes: a substrate; electrodes disposed on the substrate and operable to detect and/or perform energy harvesting from an analyte in sweat; and a sweat permeation layer having a first side and a second side located opposite to the first side, wherein the first side of the sweat permeation layer is in contact with the electrodes such that the electrodes are disposed between the substrate and the first side of the sweat permeation layer, and wherein the sweat permeation layer is structured to allow sweat applied to the second side permeate through the sweat permeation layer to reach the electrodes through the first side of the sweat permeation layer.

In some example embodiments, the electrodes are a part of one of: an electrochemical sensor, and affinity-based sensor, and optical sensor, a catalytic/biocatalytic fuel cell. In some example embodiments, the sweat permeation layer includes at least a layer of a hydrogel. In some example embodiments, the hydrogel includes at least one of: polyvinyl alcohol (PVA), poly acrylic acid (PAA), polyethylene oxide (PEO), polyacrylamide (PAM), cellulosic materials (e.g., cellulose, methylcellulose, ethylcellulose, hydroxyethylcellulose), agar, gelatin, agarose, alginate, glycerol, ethylene carbonate, propylene carbonate; wherein the hydrogel can be disposable after each use or reused, with a corresponding container for the storage and the placement. In some example embodiments, the analyte is glucose, and the electrodes include an electrochemical sensor comprising a reference electrode, a working electrode, and a counter electrode, wherein the reference electrode includes silver, and wherein the working electrode includes Prussian blue and glucose oxidase. In some example embodiments, the analyte or the fuel is lactate, and the electrodes includes an electrocatalytic anode and a cathode, wherein the cathode includes catalysts that can facilitate oxygen reduction reaction, or a oxidative material that itself can be reduced including silver oxide, nickel oxide, manganese oxide, and wherein the anode electrode includes lactate oxidase and reaction mediators such as tetrathiafulvalene (TTF), naphthoquinone (NQ), ferrocene and its derivatives (e.g. methylferrocene, dimethylferrocene), or the complex of such (e.g. tetrathiafulvalene tetracyanoquinodimethane). In some example embodiments, an electrode in the electrodes is constructed with a large thickness and high porosity, wherein the electrode is constructed with carbonaceous materials including graphite, carbon black, carbon nanotubes, or graphene, and wherein the electrode includes an elastomeric binder including styrene-based triblock copolymers (e.g. polystyrene-polyisoprene-polystyrene, poly styrene-polybutylene-polyethylene-polystyrene), fluorinated rubbers (e.g. poly (vinylfluoride-tetrafluoropropylene)), polyethylene vinyl acetate, polyurethane, Ecoflex, or Polydimethylsiloxane, and wherein the construction of the electrode includes a template particle that will be thereafter removed via dissolution or etching, including salt (e.g. sodium chloride, sodium bicarbonate), sucrose, metal (e.g. Mg, Zn), or polymers (e.g. styrene), and wherein the electrode includes redox reaction active materials including conductive polymers (e.g. poly(3,4-ethylenedioxythiophene) polystyrene sulfonate), 2-D materials (e.g. Molybdenum disulfide), two-dimensional inorganic compounds, e.g., having layers of a few atoms thick, such as MXenes (e.g. Ti₂C₃). In some example embodiments, an electrode in the electrodes is constructed with the electrodeposition of a conductive polymer including polypyrrole, polyethylenimine, and polyaniline by the application of a constant voltage or a voltage range scanned repeatedly for a controlled amount of time; and wherein the electrode is constructed with a redox-active material including mediators or organic dyes that is co-deposited onto the electrode during the electrodeposition of the conductive polymer; and wherein the electrode is constructed with the target analyte molecules of the sensors including cortisol, insulin, levodopa and proteins, which are thereafter eluded from the sensor electrode via applying a constant voltage, a voltage range scanned repeatedly, an aqueous solution, or an organic solution for a controlled amount of time to create a molecularly imprinted polymer electrode containing recognition cavities that can selectively bind with the sweat analytes from the finger. In some example embodiments, a device for collecting sweat for the estimation of a concentration of a blood analyte or utilization of a redox reaction of such analyte for energy generation comprise a voltage regulatory circuit, wherein the circuit increases the voltage which, when connected to the electrocatalytic electrodes, cause the input signal from the electrodes to increase and being able to be stored in energy storage devices such as a capacitor, a supercapacitor, a battery, or a combination of such. According to some example embodiments, the device includes a voltage regulatory circuit coupled to at least an electrode of the electrodes of the device and configured to harvest electric energy generated by the device and store that energy in an energy storage device.

In some embodiments of the disclosed technology, a method of generating power using the collected sweat analyte includes: placing a device according to the disclosed technology on a skin surface with sweat glands to collect the analyte for a biocatalytic reaction in the electrodes of the device to generate a current from the electrodes of the device, wherein the sweat is collected by the device from a finger of other sweat-gland covered skin through the sweat permeation layer of the device; sporadically or frequently applying pressure to the device against the skin via finger pressing to generate a current from the electrodes, collecting the energy directly or through a voltage regulatory circuit to the storage unit and to discharge such storage unit thereafter; collecting the energy directly within the highly porous electrodes of the device and discharge thereafter.

In some embodiments of the disclosed technology, a method of determining a concentration of a biofluid analyte includes: obtaining, for an individual, several measurements of a level of the analyte in sweat of the individual using a self-generated signal or an open-circuit voltage from a device according to the disclosed technology, wherein the sweat is collected by the device from a finger of the individual touching the sweat permeation layer of the device; for each measurement in the several measurements of the level of the analyte in the sweat of the individual, a voltage signal without external exertion of a constant voltage or current can be obtained by discharging via a load, usually a resistor with known resistance, between the anode and the cathode; for each measurement in the several measurements of the level of the analyte in the sweat of the individual, a discharge of the device according to the disclosed technology (BFC) resulting in discharge of energy that is regulated, stored, and/or to directly power electronics that obtain the signal from the electrodes.

In some embodiments of the disclosed technology, a method of determining a concentration of a blood/sweat/ISF analyte includes: obtaining, for an individual, several measurements of a level of the analyte in sweat of the individual using a signal from the sensor of a device according to the disclosed technology, wherein the sweat is collected by the device from a finger of the individual touching the sweat permeation layer of the device; for each measurement in the several measurements of the level of the analyte in the sweat of the individual, obtaining a measurement of a concentration of the analyte in the biofluid of the individual; obtaining an exponential power parameter, and exponential multiplier parameter, and an intercept parameter for a dependence between the obtained measurements of the concentration of the analyte in the blood of the individual and the obtained measurements of the level of the analyte in the sweat of the individual; and using the exponential power parameter, exponential multiplier parameter, and the intercept parameter to translate a new measurement of the level of the analyte in the sweat of the individual to an estimate of the concentration of the analyte in the blood of the individual.

FIGS. 9A-9C show in vitro calibration curve for the glucose sensor. FIG. 9A shows chronoamperogram for successive additions of 50 μM of glucose in 0.1M PBS pH 7 at −0.2V for 60 seconds. FIG. 9B shows calibration plot obtained from these chronoamperograms. FIG. 9C shows calibration plot of current signal vs blood glucose in mg/dl.

FIGS. 10A-10E show optimization of the hand washing step with three repeated experiments using different glucose sensor. Specifically, FIG. 10A shows touching the sensor without washing hands, FIG. 10B shows touching the sensor after 10 s washing with soap, FIG. 10C shows touching the sensor after 20 s washing without soap, and FIG. 10D shows touching the sensor after 20 s continuous swiping with wet tissue. FIG. 10E shows the bar plots for the optimization of the hand washing step.

FIGS. 11A-11F show optimization of the touching time. Specifically, FIG. 11A shows touching the sensor for 10 sec, FIG. 11B shows touching the sensor for 30 sec, FIGS. 11C shows touching the sensor for 1 min, FIG. 11D shows touching the sensor for 3 min, and FIG. 11E shows touching the sensor for 5 min. FIG. 11F shows response vs. touching time plot, and the optimal touching time is 1 minute.

FIGS. 12A-12B show stability of the personal factors, slope (a) and intercept (b) for two subjects (FIG. 12A, FIG. 12B) over a 4-week period with confidence bands of 95%.

Rationing for the Personalized Calibration Software

The initial calibration is acquired using equations 1-3 and the sweat personal values are computed in the software where the corresponding blood glucose concentration is calculated. Once or twice a month, a new validated data would be inserted in the software for the moving average calculation. This new inserted value is first validated (e.g., outlier detection) and if the value is within the expected range, it can be included in the initial calculated calibration curve to obtain the new averaged parameters. If the inserted blood value is outside the confidence interval, the value is rejected, and a new input is requested. If the software rejects three values consecutively, the software indicates the need of a whole new calibration plot (FIG. 13). This protocol ensures automation of the personal mathematical treatment and high quality for the output values. The friendly software is well accepted by the patients, showing good performance and readout values.

FIG. 13 shows a flowchart for the calibration and analysis of sweat glucose signals to blood glucose concentrations using the fingertip touch-based sensor. First, the initial calibration is acquired. Next, sweat values are computed in the software and the corresponding blood glucose concentration is calculated. Once a month, a new validated data is inserted in the software for the moving average technique implementation. In the moving average methodology, new averaged parameters are calculated using new and previously loaded data. For such, the user must input a blood value corresponding to the sweat reading. The blood value is initially validated (e.g., outlier detection) by the software and if the value is withing the expected range, it is included in the initial calculated calibration curve and new averaged parameters are obtained. If the inserted blood value is not within the expected range, another value is requested, if the input is rejected three times by the software, a whole new calibration must be realized. This protocol ensures automation for the mathematical treatment and quality for the output values.

FIG. 14 shows box plots for mean absolute relative difference (MARD) on successive measurements during the day for three subjects. Displayed are mean (diamonds), median (horizontal lines within boxes), 25th and 75th percentiles (lower and upper edge of the boxes), and minimum and maximum values (Whiskers).

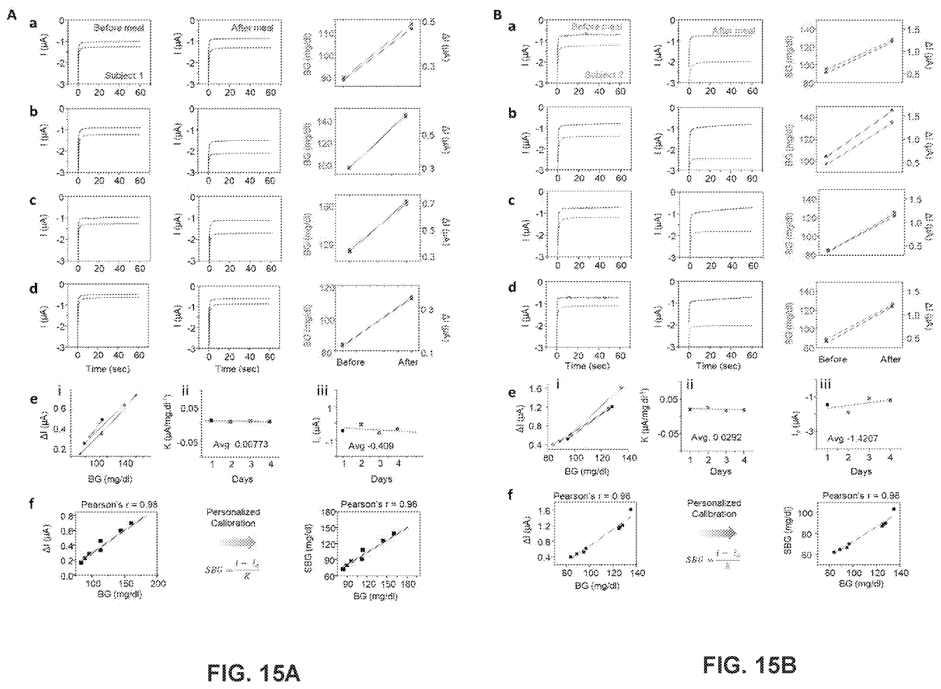

FIGS. 15A-15B show an example data processing protocol for personalized transduction equation. Sweat glucose is monitored for two subjects for four days, twice a day. Prior to each analysis, a commercial blood glucose meter is used to measure the blood glucose values of the user. The individual values of the signal from sweat for each day is correlated with the blood values generating a linear plot with specific slope and intercept values (a-d). The slopes and intercepts obtained for each day are then averaged and a personalized equation is generated for each user (e-i-iii). Upon obtained such personal transduction equation, the current signal from the glucose in sweat is used for the direct translation of the signal to blood glucose values.

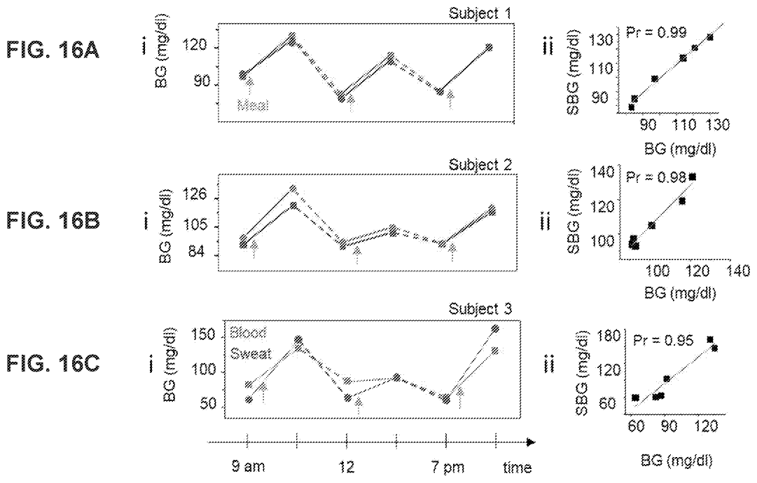

FIGS. 16A-16C show a whole day sweat glucose determination. Glucose levels in sweat collected from the fingertip using the touch sensor device during the whole day after three meals. The signal obtained from the sweat sensor is directly translated to blood glucose levels using the personalized translation equation from each user. The correlation and Pearson's r values are shown in (ii).

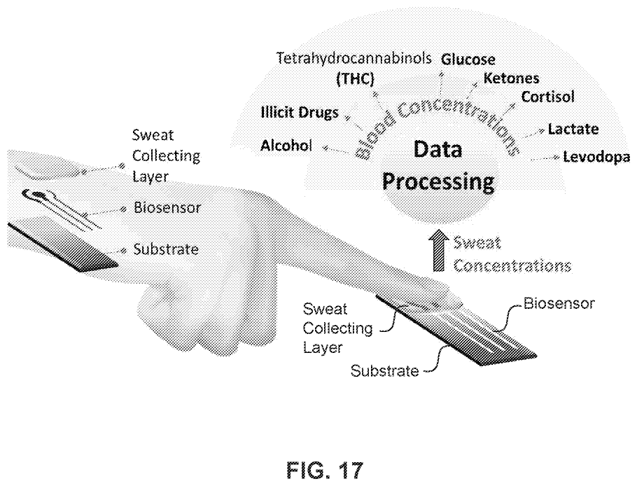

FIG. 17 shows an application of the fingertip sweat sensor. In some implementations, the applications of the data processing methodology can be combined with several biosensors, including but not limited to levodopa biosensor, modified via tyrosinase enzyme or non-enzymatic sensor via voltametric techniques, lactate biosensor modified via lactate oxidase enzyme (or other recognition elements), cortisol biosensor modified via molecularly imprinted polymerization (MIP) (or other recognition elements), ketones bodies biosensors using β-Hydroxybutyrate dehydrogenase enzyme modified sensors (or other recognition elements), glucose biosensor using glucose oxidase enzymes (or other recognition elements), THC sensors using either nanoparticle, CNTs or MIP modified sensors (or other recognition elements), illicit drugs such as cocaine using bare carbon electrode (or other recognition elements), and alcohol using the enzyme alcohol oxidase (or other recognition elements).

The disclosed technology can be implemented in some embodiments to provide a data processing approach for correlating sweat analyte response of biomarkers in natural passive perspiration and their blood concentrations. The new algorithm addresses inter-individual variability for accurate translation to blood values of these biomarkers. Such new personalized data processing is combined with a touch-based fingertip sweat analysis. A glucose oxidase-based biosensor is used for measuring sweat glucose and a molecular imprinted polymer (IP) based sweat sensor device for cortisol monitoring. The sweat collection device includes a biosensor realized by screen-printing, sputtering, inkjet or any other appropriated sensor fabrication technique, covered by a sweat collecting layer comprising but not limited to a hydrogel such as PVA, agarose or glycerol. Passive sweat is collected from the skin upon direct contact with the sweat collecting layer. After contacting the skin for a determined amount of time, the collected sweat diffuses through the hydrogel layer, reaching the recognition layer, where the analyte is measured. Several sensing techniques can be used for the analyte determination including but not limited to electrochemical, affinity, and optical sensors. After data acquisition the personalized correlation equation can be determined. For this, data is acquired for several days and validated with appropriated approaches. For example, the determination of sweat glucose can be validated using commercial blood glucometer. Blood sample is collected and analyzed prior each measurement for the validation steps. After data collection, the linear slope and intercept obtained each day is averaged and a personalized universal equation is derivate for direct conversion of the signal intensity to the blood concentration. The demonstration of such device and data processing is realized by measuring glucose levels in sweat collected from the fingertip. As discussed above, the working electrode of a screen printed 3-electrode system is modified with the enzyme glucose oxidase and a Polyvinyl alcohol (PVA) hydrogel is placed over the modified sensor to serve as the sweat collector layer. Sweat is collected from the fingertip during, e.g., 1-minute touching after proper washing of the hands. After collection, sweat glucose signal is obtained by chronoamperometry. The signal is obtained twice a day for one week and validate against a commercial blood glucometer. As discussed above, a linear correlation between the two points (sweat and blood glucose) is obtained for each day of analysis and an averaged slope and intercept is calculated for the user. These personalized values account for the individual sweat parameters such as sweat rate and composition. The personalized general equation is then used to direct translate the sensor signal into blood glucose values. Moreover, the advantage of this methodology can be expanded to access analytes from the fingertip sweat, such as levodopa, lactate, alcohol, illicit drugs, tetrahydrocannabinol (THC), and ketones bodies by simply modifying the electrode surface that suffices to the analyte.

As discussed above, sweat cortisol levels are also measured by touching the PVA gel with fingertip for 30 sec after 2 min of washing hands. The cortisol sensor comprises the molecular imprinted polymer (IP) layer containing the signal indicator (e.g., any materials that has redox characteristics such as Prussian blue, ferrocene, methylene blue, or else) and cavity for cortisol detection, promoting a label free MIP sensor, which does not need for additional external signal indicator for the measurement with high selectivity. The current response can be measured using chronoamperometry after 2 min of incubation time to have the binding process between the MIP layer and cortisol.

The disclosed technology can be implemented in some embodiments to provide a new treatment for sweat-to-blood signal translation. In some implementations, an application of the new methodology uses a fingertip sweat sensor for glucose or cortisol monitoring. Current sweat sensors rely on extensive exercising, heat or chemical stimulation for sampling sweat, these current protocols demand time, energy and power consumption. The disclosed technology relies on the processing of the signal obtained by the collection of passive natural sweat without the need of performing exercising or any additional sweat stimulation steps. Sweat is collected when the collecting hydrogel, located over the sensing area, is in contact with the skin, the collected sweat diffuses through the gel to the sensor, where sweat analytes are measured. In some implementations, the feasibility of the mathematical application by collecting sweat from the fingertip upon touching. Sweat glucose and cortisol is measured by chronoamperometry, the total time for the analysis is 2 minutes, including 1 minute sweat sampling and 1 minute sweat detection. The new data processing ensures that personal differences in sweat rate or skin properties are accounted for. Previous work brings conflicting discussion about correlation of sweat analytes (glucose, cortisol, lactate, etc.) and blood concentrations. The divergence in previous results is mostly correlated with the sweat collection steps and the data processing of the results. In some implementations, a methodology for sweat analysis includes the collection, sensing, and processing steps. The disclosed technology can be implemented in some embodiments to provide a reliable non-invasive option for the frequently monitoring of analytes such as levodopa, glucose, ketones bodies, lactate, alcohol, illicit drugs, tetrahydrocannabinol (THC), and cortisol. The existing commercial glucose meter requires a finger prick blood testing protocol which is invasive and is inconvenient and painful for repeated frequent testing. The new touch-based glucose test allows such frequent glucose measurements and obviate the need for periodic blood measurements and validations. The simplicity and speed of the new touch-based blood-free fingertip assay hold considerable potential for reliable frequent self-testing of glucose towards improved management of diabetes. On the other hand, there is no commercially available test for cortisol detection. Our method can easily translate the detect the glucose and cortisol levels in sweat to blood glucose values by simple touching with fingertip that does not need any invasive and sweat inducing protocol. For this, data is acquired daily and validated with appropriated approaches. For example, the determination of sweat glucose can be validated using commercial blood glucometer and cortisol can be validated using affinity tests (immunosensors). The initial data collection is used for estimating the personal slope and intercept, and these personal factors can be used over several weeks without the need for parallel blood testing. A personalized universal equation is thus used for direct conversion of the sweat signal intensity to the blood concentration (FIGS. 15A-15B).

The disclosed technology can be implemented in some embodiments to provide a new methodology that can be used to translate sweat biomarker measurements to reliable estimate of blood concentrations based on personalized data processing accounting for inter-individual variability. For this a non-invasive touch-based sweat sensor is used to measure sweat analytes. A biosensor covered by a sweat collection layer is used for determining sweat analytes in natural sweat (FIGS. 1A-1F). A screen-printed electrochemical sensor is modified with the enzyme glucose oxidase or the cortisol imprinted layer and covered with a layer of PVA hydrogel. The hydrogel layer is able to collect natural sweat upon contact with the body. Sweat from the fingertip is used in the analysis. Upon touching, sweat from the finger accumulates in the hydrogel during a fixed time and it diffuses through the PVA gel reaching the sensing layer of the electrode where it is measured. Chronoamperometry is used to monitor the glucose and cortisol concentration in the fresh collected sweat. The signal obtained for each user is then treated in order to build a personalized calibration for recovering the blood glucose and cortisol values. For the personalized calibration, prior to the testing, a commercial blood glucose meter or immune assay tests are used to measure the blood glucose and cortisol values of the user, respectively. Next, each user tested their sweat glucose or cortisol levels using the device. This procedure is repeated twice a day, for several days, and the individual values of the signal from sweat for each day is correlated with the blood values generating a linear plot with specific slope and intercept values. The slopes and intercepts obtained for each day are then averaged and a personalized equation is generated for each user (FIGS. 15A-15B). Upon obtained such personal transduction equation, the current signal from the analyte in sweat can be directly translated to blood glucose values (FIGS. 16A-16C). A simple software using moving average calculation can be implemented on the electronics for autonomous data treatment (FIG. 13). First, the initial calibration is acquired. Next, sweat values are computed in the software and the corresponding blood glucose concentration is calculated. Once a month, a new validated data is inserted in the software for the moving average technique implementation. In the moving average methodology, new averaged parameters are calculated using new and previously loaded data. For such, the user must input a blood value corresponding to the sweat reading. The blood value is initially validated (e.g., outlier detection) by the software and if the value is withing the expected range, it is included in the initial calculated calibration curve and new averaged parameters are obtained. If the inserted blood value is not within the expected range, another value is requested, if the input is rejected three times by the software, a whole new calibration must be realized. This protocol ensures automation for the mathematical treatment and quality for the output values. This new sweat platform and correlation methodology can be translated for the analysis of any sweat biomarker (such as, but not limited to levodopa, lactate, alcohol, illicit drugs, tetrahydrocannabinol (THC), ketones bodies, and cortisol) FIG. 17. More complex analysis can be performed by loading different reagents in the sweat collection gel itself. Sweat from different parts of the body can also be collected using a wearable epidermal platform for the device such as tattoos, textiles, or accessories (watches, headband, eyeglasses, etc.). Different hydrophilic hydrogels can be used as the sweat collection layer as long as their morphology allows rapid diffusion and stability.

The disclosed technology can be implemented in some embodiments to provide diabetes management methods and devices. Diabetes prevalence has been exponentially rising increasing the needs for extensive research on non-invasive approaches for glucose monitoring. Candidates to replace the current blood fingerstick glucose sensor include biosensors based on saliva, tears, sweat and ISF as surrogate for blood. Among these biofluids, sweat has been receiving greater attention due to its favorable composition and easiness of access. However, even though several sweat glucose sensors have been published, there are mixed reports on the correlation of sweat and blood glucose levels. The disclosed technology can be implemented in some embodiments to provide a new combination of finger sweat sampling sensor with a simple algorithm for the translation and normalization of sweat-to-blood glucose values. The non-invasive nature of finger sweat analysis increases patient compliance promoting better glucose management besides eliminating changes in sweat properties related to the different sweat collection methodologies. Thus, a finger sweat touch-based glucose sensor can be used to measure sweat glucose from diabetes patients and blood validated values can be used to generate a personalized equation for the signal translation, with largely different slope and intercept values obtained for different subjects and reflect their distinct sweat rate, composition, and skin properties. Such personal variations among individuals are related with age, gender, or race. Once the personalized conversion is established and is used for training the system and processing future results. Such system training leads to substantially improved accuracy with Pearson correlation coefficient (Pr) higher than 0.95, and overall mean absolute relative difference (MARD) of 7.79%, with 100% of paired points residing in the A+B region of the Clarke error grid (CEG). The glucose detection protocol leverages the fast sweat rate on the fingertip for rapid glucose assays ofnatural perspiration without the need for physical activity or iontophoretic or heat sweat stimulation protocols, and the new personalized sweat-to-blood translation allows to correlate different sweat constituents eliminating variables such as sweat rate, composition, and skin type.

The disclosed technology can be implemented in some embodiments to provide drug detection methods and devices. Driving under the influence of illicit or licit drugs such as cannabis and alcohol represents one of the major safety concerns due to the strong synergistic effect of these substances. Therefore, a rapid in-situ testing of such substances is needed to decrease the risks of road accidents. Thus, the disclosed technology can contribute to the accurate and fast decentralized, detection of drugs using finger sweat sensor combined with the mathematical approach. The disclosed technology can be used as a personal safety system for car ignition where the finger sweat sensor is directly integrated to the car's ignition, including but not limited to the on/off button, the car's keys, etc. Multiple sweat drug molecules can be detected simultaneously for drug screening and identification. The software used for personalized quantification of such drugs can include a drug data base for identifying the substance in sweat. The disclosed technology can promote such important and needed application for self-monitoring towards safety, besides enabling law enforcement personnel to screen drivers during traffic stop, addressing the growing concerns of drug-impaired driving.

The disclosed technology can be implemented in some embodiments to provide sweat biomarker monitoring methods and devices. The personalized processing of touch-based fingertip sweat assays offers simplified accurate tracking of key sweat biomarkers, such as levodopa, cortisol, alcohol, lactate, ketone bodies, or uric acid as well as illicit drugs or tetrahydrocannabinol (THC). Tracking cortisol level fluctuations is important in understanding the body's endocrine response to stress stimuli. Traditional cortisol sensing relies on centralized laboratory settings, while wearable cortisol sensors are limited to slow and complex assays. The disclosed technology can be implemented in some embodiments to provide a simple touch-based molecularly imprinted polymer (MIP) sensor for rapid cortisol detection. The sensor readily samples natural sweat from the fingertips onto the cortisol-imprinted polypyrrole, with embedded Prussian blue redox probes, obviating the need for stressful and lengthy sweat-extraction procedures. By eliminating time lags, such rapid (3.5 min) fingertip assay enables capturing of sharp variations in cortisol levels compared to previous methods. Such advantages are demonstrated by tracking cortisol response throughout day-long circadian rhythm, along with gold-standard immunoassay validation. The rapid touch-based cortisol sensor offers an attractive, accessible, stress-less avenue for quantitative stress management.

While current methodologies for sweat glucose and cortisol analysis involve either exercising of artificial sweat stimulation, the disclosed technology offers a fast, safe, and reliable methodology for sweat collection, measurement (IP), and personalized data processing. Correlating values of sweat biomarkers with the corresponding blood values is current a challenge for the sweat sensor industry, the new methodology disclosed here makes possible to account for the inter-individual variability for accurate estimate of the blood concentration.

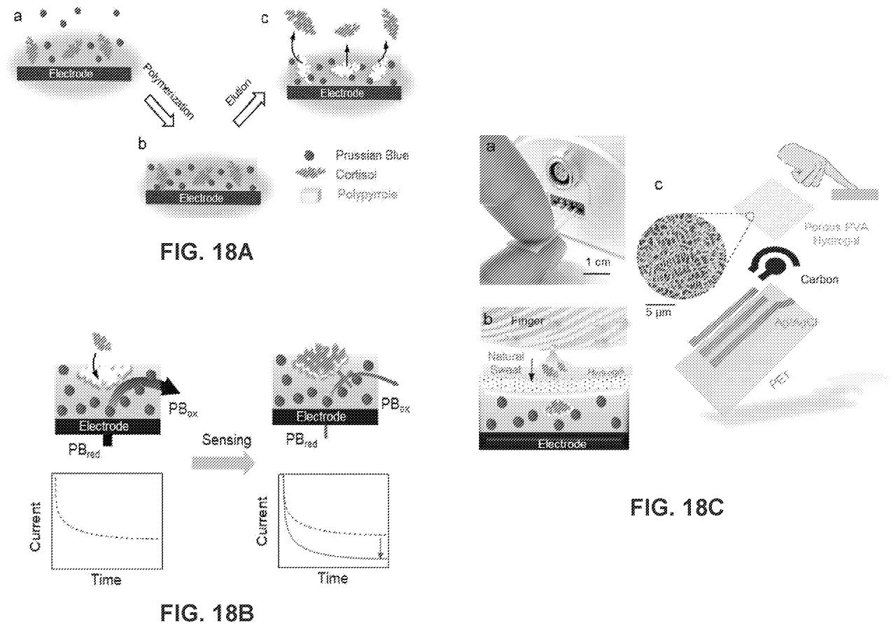

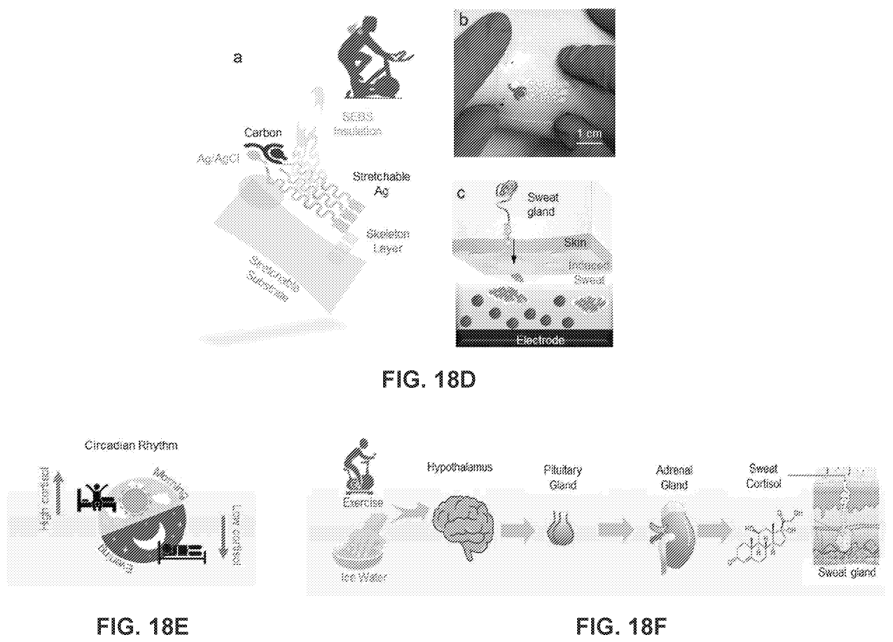

FIGS. 18A-18F show an example of molecular imprinted polymer (MIP)-based sensor for rapid, stressless cortisol sensing. FIG. 18A shows synthesis of the MIP layer for cortisol sensing: (a) PB, cortisol, and pyrrole are co-electrodeposited onto the printed carbon electrode; (b) the entrapped cortisol template is eluted from the polymerized PPy; (c) the corresponding MIP recognition layer after the cortisol elution, where cortisol-specific cavities are formed in the electrode. FIG. 18B shows the sensing mechanism of the MIP. The anodic current from the oxidation of the embedded PB is decreased after the binding of cortisol to the MIP. FIG. 18C shows the touch-based fingertip cortisol sensor, with: (a) photos demonstrating the single-touch sensor application; (b) illustration of the sensing mechanism, where the cortisol from the accumulated finger sweat diffuses through the hydrogel onto the MIP electrode; and (c) structural illustration of the fingertip cortisol sensor, with the cryogenic scanning electron microscopy (cryo-SEM) image of the porous PVA hydrogel (inset). FIG. 18D shows the stretchable epidermal cortisol patch, with: (a) the structure of the stretchable sensor, which the sensor adapts stretchable interconnection and substrate, shape-confining skeleton layers, and stretchable polymer insulation; (b) a photo demonstrating the usage of the patch on the skin after generating sweat from an exercise session; and (c) the sensing mechanism of the epidermal cortisol patch, where the sweat directly interact with the MIP electrode. FIG. 18E shows the fluctuation of cortisol through the circadian cycle. FIG. 18F shows the induction of cortisol secretion through acute physical stimulations.

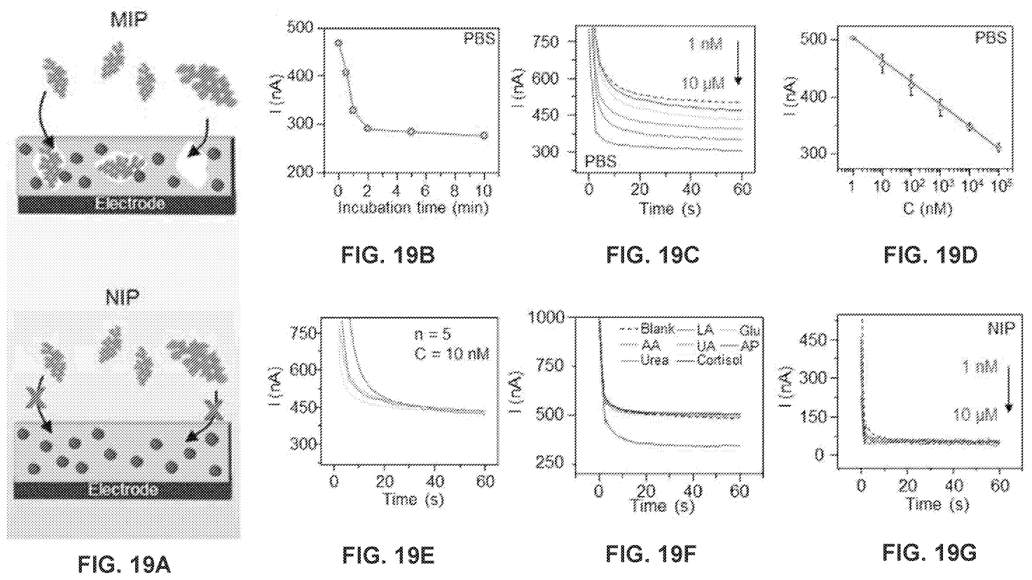

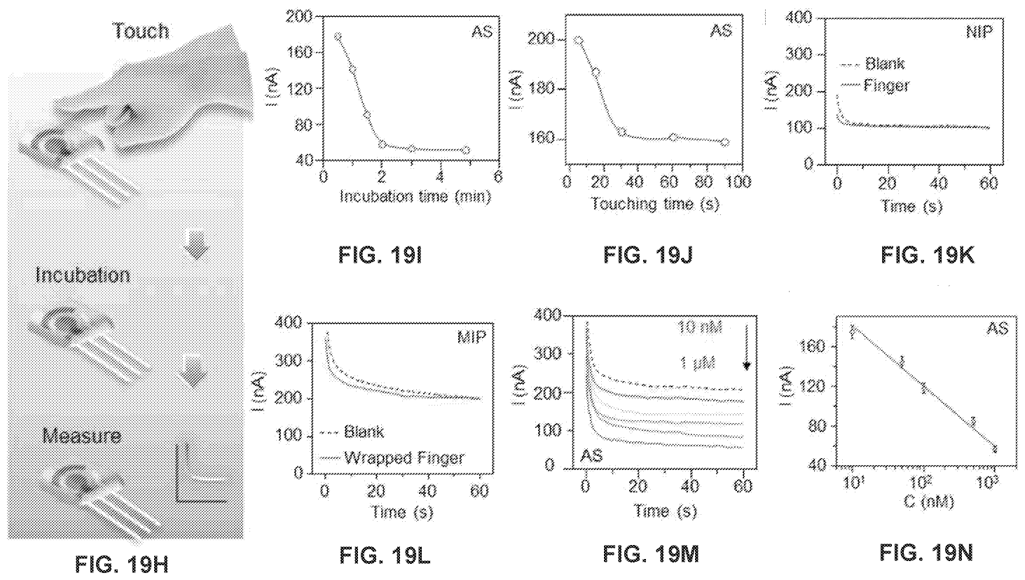

FIGS. 19A-19N show optimization and calibration of the MIP cortisol sensing in various media. FIG. 19A shows the interaction of cortisol in the MIP electrode compared to the lack of interaction in the NIP electrode. FIG. 19B shows the optimization of the incubation time prior to sensing in PBS medium. 2-min incubation is determined to be the most efficient and accurate incubation time for the cortisol to interact with the MIP electrode. FIG. 19C shows electrochemical response of the MIP sensor to different cortisol concentrations in PBS. FIG. 19D shows the corresponding calibration curve, showing a logarithmic response of the electrode current to the cortisol within the detection limit, FIG. 19E shows the overlaid CA of five sensors' response to 10×10⁻⁹ m cortisol, demonstrating the reproducibility of the fabricated cortisol sensor. FIG. 19F shows response of the MIP cortisol sensor in PBS to the addition of lactic acid, glucose, ascorbic acid, uric acid, acetaminophen, urea, showing no change in response, followed by the addition of 1×10⁻⁶ m of cortisol, which shows a clear response. FIG. 19G shows response of the NIP-based sensor to different concentrations of cortisol in PBS. FIG. 19H shows the one-touch cortisol sensing procedure. The finger is placed on the MIP sensor covered by an AS-based PVA hydrogel for 30 s, followed by 2 min incubation time, and the measurement. FIG. 19I shows optimization of the incubation time for cortisol to interact with the PVA hydrogel-covered MIP sensor in AS. FIG. 19J shows optimization of the touching time of the finger on the gel in the one-touch cortisol sensing procedure. FIG. 19K shows response of the touchedNIP electrode showing no response to the sweat on the finger. FIG. 19L shows the CA of the MIP electrode touched by a covered finger showing no sensor response to the mere pressing movement. FIG. 19M shows the CA response of the MIP cortisol sensors with AS-based PVA hydrogel with different cortisol concentrations. FIG. 19N shows the corresponding calibration curve, showing a logarithmic current-concentration dependence based on the signal.

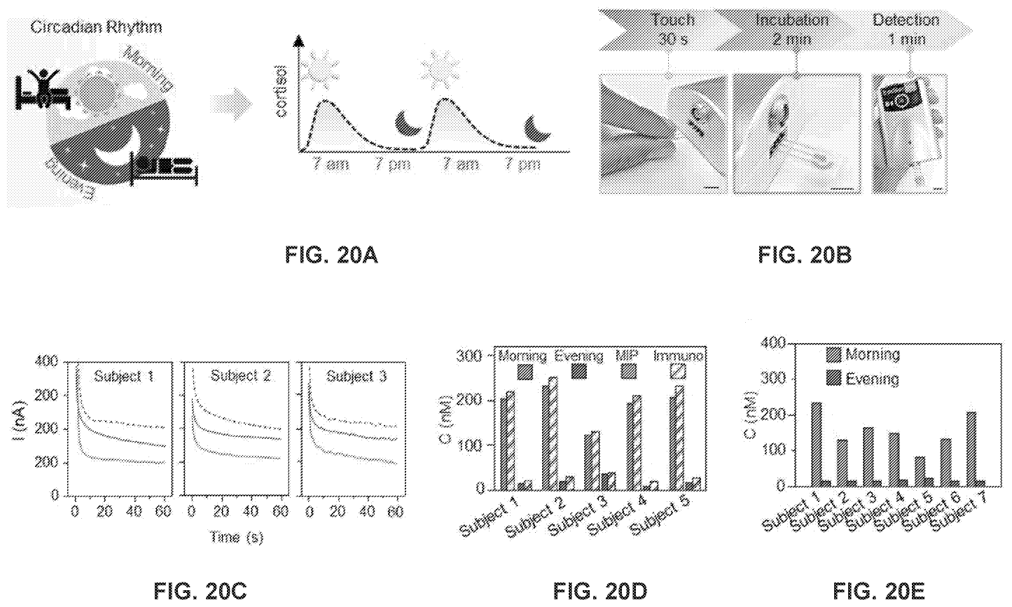

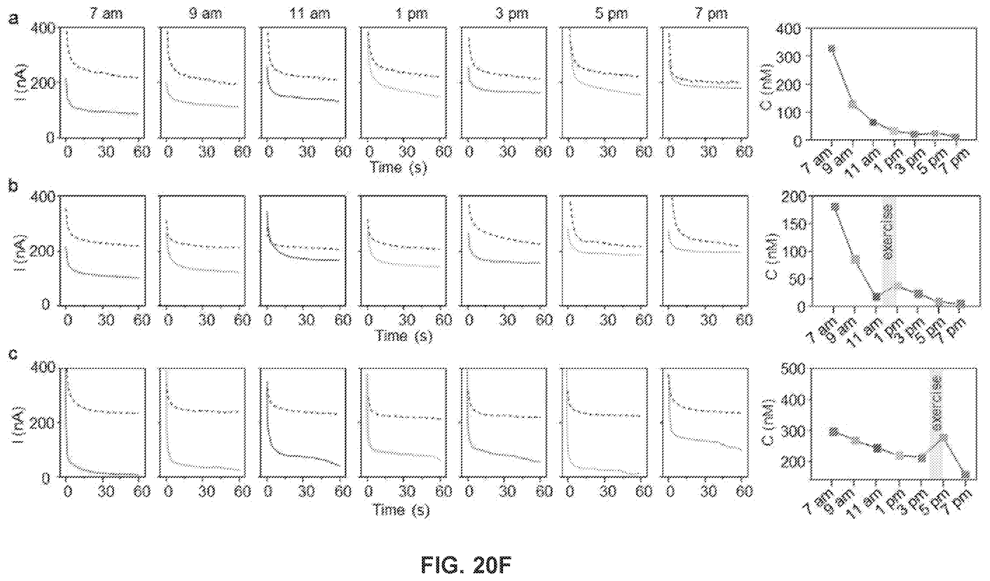

FIGS. 20A-20F show an example of endogenous cortisol monitoring. FIG. 20A shows cortisol levels with circadian rhythm. Cortisol levels are found to be higher during the morning, decreasing at night. FIG. 20B shows a protocol used for sweat finger analysis. Sweat is collected during 30 s in the collection gel, next, 2 min incubation time is allowed for MIP interaction with analyte followed by the signal acquisition using a handheld potentiostat (scale bar: 1 cm). FIG. 20C shows CAs of sweat cortisol response on the MIP modified electrode for subjects 1-3. The black solid line corresponds to the sweat collection gel background, the red line is the cortisol signal measured in the morning and the blue line corresponds to the signal measured in the evening. FIG. 20D shows a validation of the electrochemical signal (solid colors) obtained from the sweat finger sensor (red is the signal obtained during the morning and blue during the evening), and the immunosensor response (hatched bars) using sweat collected by pilocarpine IP stimulation. FIG. 20E shows a cortisol response during morning and evening obtained using the finger sweat sensor for seven patients. FIG. 20F shows continuous cortisol monitoring during the day using the finger sweat sensors on three subjects. Subjects b and c included a 30 min exercising routine (indoor biking) at 1 and 5 p.m., respectively.

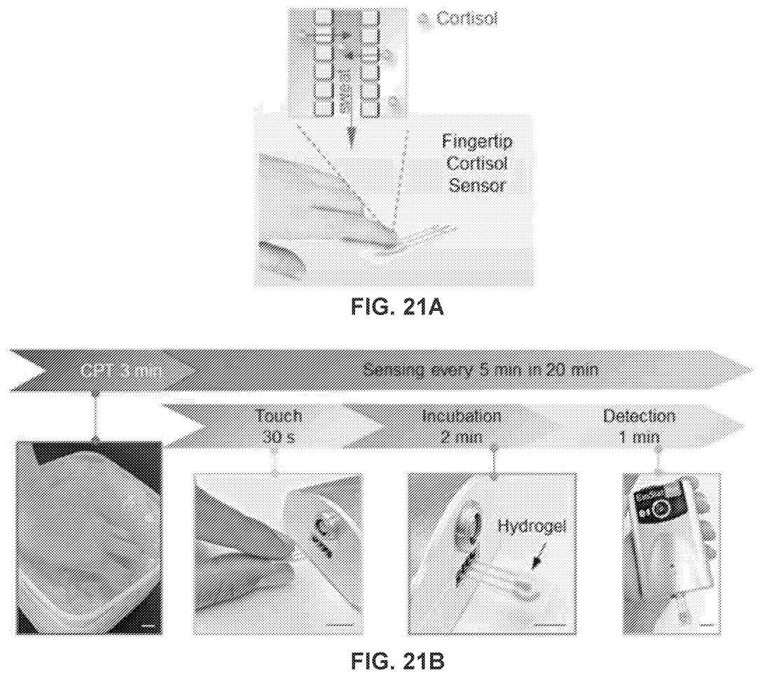

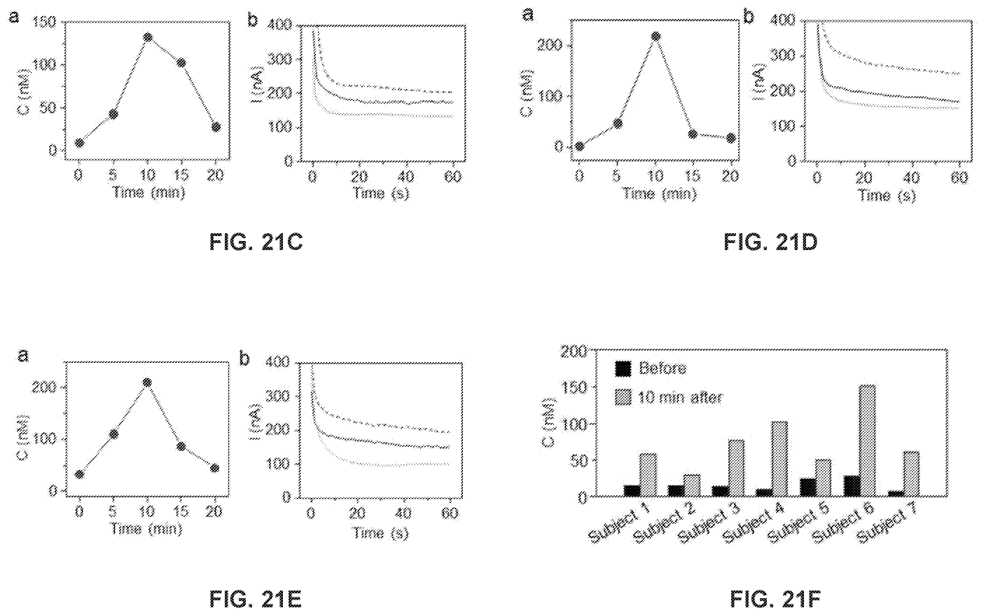

FIGS. 21A-21F show an example of cortisol sensing during acute stimulation via CPT. FIG. 21A shows the release of cortisol in the natural sweat from the fingertip sweat pores to the hydrogel. FIG. 21B shows the timeline of the sensing sequence during the ice-water CPT stimulation. The hand of the subject is submerged in ice water for 3 min, while the other hand of the subject is sampled using the touch-based cortisol sensor every 5 min (scale bar: 1 cm). FIGS. 21C-21E show the change in cortisol concentrations of three subjects during the 20 min after the CPT, showing that (a) in general, the cortisol level peaks at the 10 min mark, and (b) their corresponding amperograms as blank, 0 min and 10 min after the CPT. FIG. 21F shows the change in cortisol level in 7 subjects at 0 and 10 min after the CPT stimulation.

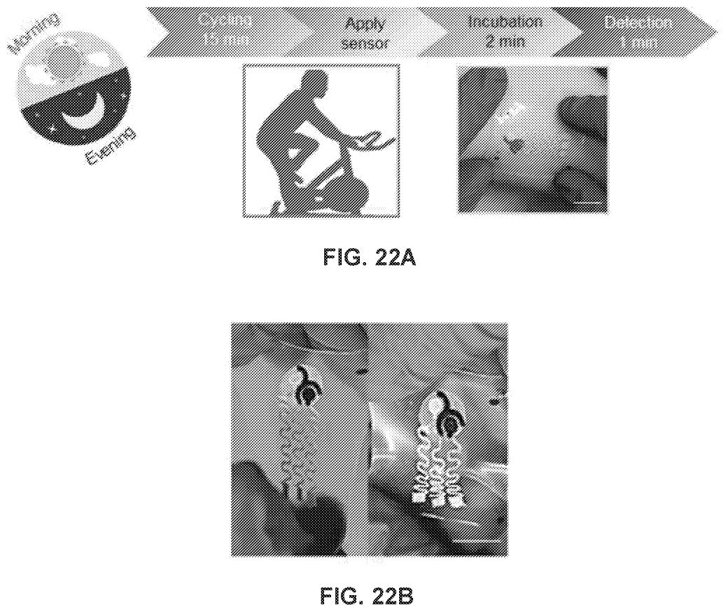

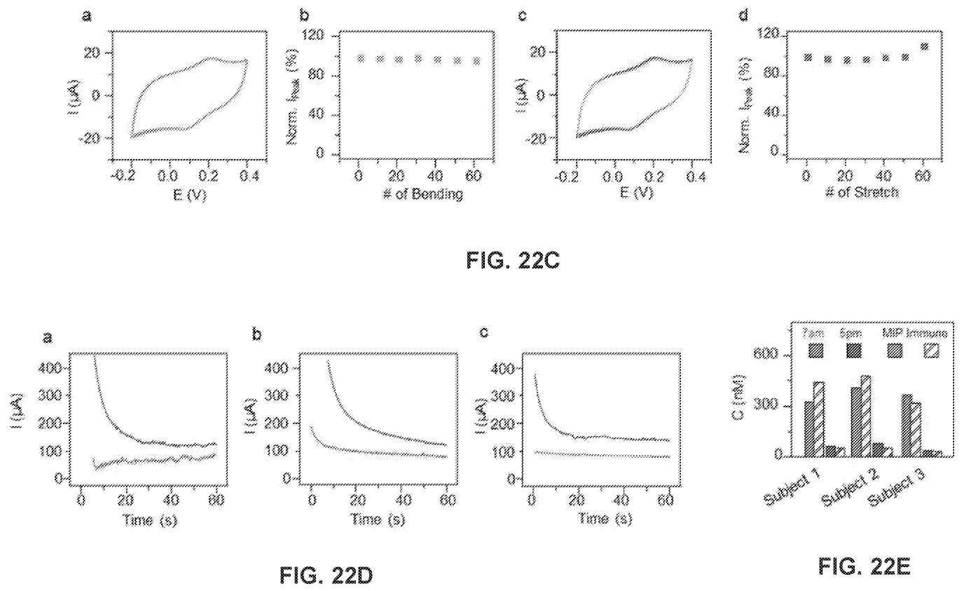

FIGS. 22A-22E show an example of on-body cortisol detection using the wearable sensor patch. FIG. 22A shows the schematic illustration of the adapted protocol used for on-body testing, relying on applying the sensor onto the forearms of subjects after a 15 min indoor cycling exercise, followed by 2 min incubation and 1 min CA (scale bar: 1 cm). FIG. 22B shows the designed stretchable wearable electrode during stretching and bending (scale bar: 1 cm). FIG. 22C (a)-(d) show CV and the corresponding peak currents of the cortisol sensor patch in 1.0 mm [Fe(CN)₆]^{3-/4-} while undergoing repeated bending (FIG. 22C (a), (b)) and stretching (FIG. 22C (c), (d)). FIG. 22D shows CA response of the sensor for on-body detection of sweat cortisol in three subjects (a-c) at 7 a.m. (red curves) and 5 p.m. (blue curves). FIG. 22E shows the correlation between IMIP-based wearable cortisol sensor and the immunosensor for the detection of cortisol concentration in human sweat.

The disclosed technology can be implemented in some embodiments to provide a touch-based stressless cortisol sensing methods and devices.

Tracking fluctuations of the cortisol level is important in understanding the body's endocrine response to stress stimuli. Traditional cortisol sensing relies on centralized laboratory settings, while wearable cortisol sensors are limited to slow and complex assays. Here, a touch-based non-invasive molecularly imprinted polymer (MIP) electrochemical sensor for rapid, simple, and reliable stress-free detection of sweat cortisol is described. The sensor readily measures fingertip sweat cortisol via highly selective binding to the cortisol-imprinted electropolymerized polypyrrole coating. The MIP network is embedded with Prussian blue redox probes that offer direct electrical signaling of the binding event to realize sensitive label-free amperometric detection. Using a highly permeable sweat-wicking porous hydrogel, instantaneously secreted fingertip sweat can be conveniently and rapidly collected without any assistance. By eliminating time lags, such rapid (3.5 min) fingertip assay enables the capture of sharp variations in cortisol levels, compared to previous methods. Such advantages are demonstrated by tracking cortisol response in short cold-pressor tests and throughout day-long circadian rhythm, along with gold-standard immunoassay validation. A stretchable epidermal MIP sensor is also described for directly tracking cortisol in exercise-induced sweat. The rapid touch-based cortisol sensor offers an attractive, accessible, stressless avenue for quantitative stress management.

Cortisol is a steroid hormone, released by the human body in response to psychological and physiological stress, and hence plays a major role in the body's stress response and in regulating metabolism and immune response. Chronic stress, reflected by high cortisol levels, is associated with high risks of anxiety, depression, cardiovascular diseases, and weakening immune response. Effective, rapid, and reliable cortisol detection is thus extremely valuable for dynamic stress-response profiling toward comprehensive self-monitoring, wellness management, and personalized healthcare. In a fast-evolving world, where personal wellness becomes the center of attention, simple fast decentralized testing, and non-invasive monitoring of cortisol are critical for providing guidance for personal stress management.

Cortisol can be found in various biofluids, including saliva, blood, urine, sweat, and interstitial fluids. Traditional detection of cortisol in these biofluids, carried out in centralized laboratory settings, relies on competitive immunoassays between the target cortisol and the enzyme-tagged analyte, followed by optical or electrochemical measurements of the enzymatic reaction product. While providing high sensitivity, such multi-step, complex, and lengthy immunosensing procedures are hardly adaptable for decentralized settings or wearable applications. Among the cortisol-containing biofluids, sweat and saliva are the most accessible ones. However, compared to saliva, sweat does not exhibit major matrix and biofouling effects. Accordingly, recent efforts have demonstrated the translation of immunosensors for decentralized sweat cortisol sensing, including the ability to track the cortisol diurnal cycle. Yet, such competitive immunoassay approach includes 5 min sweat stimulation, 15 min competition time, along with tagging and washing steps which are not practical for personalized, hassle-free cortisol monitoring. Label-free impedimetric immunoassay detection has also been proposed toward wearable cortisol sensing, based on monitoring the cortisol-biding induced inter-facial changes. The limited stability and high costs of cortisol antibody bioreceptors and enzyme-tagged cortisol represent another challenge to wearable and decentralized cortisol immunoassays. Artificial receptors, based on molecularly imprinted polymers (MIP), have been shown useful toward selective recognition of sweat cortisol. Yet, such MIP-based sensing commonly involves the addition of external redox signaling probes, for example, ferrocyanide which limits their on-body operation. These earlier sweat cortisol sensing protocols require lengthy physical activity or additional chemical iontophoretic (IP) sweat stimulation procedures for generating and collecting sweat. Such sweat generating methods can be disruptive to the user's daily workflow and may induce psychological or physiological stress which alters the cortisol level, resulting in inaccurate stress assessment. Therefore, the sweat collection step for cortisol monitoring represents a major barrier that hinders the development of simple, rapid, and accurate sweat cortisol sensing. Given the challenges, there are urgent needs for new stress-free non-invasive rapid cortisol sensing strategies.

The disclosed technology can be implemented in some embodiments to provide an effective novel stress-free cortisol sensing platform that allows fast, reliable, and simple detection of cortisol in sweat via a fingertip touch. Natural perspiration has been recently shown to be advantageous for sweat sampling compared to commonly used active sweat stimulation methods (exercise, heat, and IP). Unlike other body locations, the fingers—with the highest density of eccrine sweat glands—are able to generate high sweat volumes. The disclosed technology can be implemented in some embodiments to leverage such natural sweat sampling method to develop a new stress testing platform, relying on the highly scalable screen-printed electrode modified with a selective MIP recognition layer. The simple, rapid, and user-friendly cortisol sensing is realized through a series of material innovation. In order to rapidly and effectively collect sweat from the fingertip, a highly porous, permeable, and sweat absorbing polyvinyl alcohol (PVA) hydrogel is developed using sucrose as a water-soluble template to create a porous network (FIG. 18C (c) inset). Compared to the non-porous hydrogel, such templated porous hydrogel demonstrated superior permeability and a lower impedance. Natural perspiration is thus readily collected via simple fingertip touch, ensuring that only the endogenous cortisol level is measured, compared to exercise-based contrasting sweat cortisol sensors. To enable the one-step, rapid, reproducible, highly sensitive, and selective cortisol sensing, electropolymerized polypyrrole (PPy) MIP electrodes are synthesized in the presence of cortisol as the template, along with Prussian blue (PB) as the embedded redox probe, hence obviating the need for complex labeling procedure or external redox probes. The subsequent elution of cortisol from the membrane is achieved via overoxidation of PPy, which induces a structural change in the polymer that releases the template cortisol molecule (FIG. 18A). This change is confirmed using various surface characterizations and molecular simulations. The template elution resulted in surface recognition cavities that are complementary to the shape and size of the target cortisol molecule. The incorporation of PB within the MIP PPy network leads to a “built-in” electrochemical signaling probe that obviates the need for external redox probes, and hence greatly simplifying the on-body testing compared to common MIP sensors based on such solution-phase redox probe. The resulting user-friendly cortisol sensor, integrating the MIP recognition and the built-in PB-transduction element, thus relies on chronoamperometric measurements (CA) of the PB oxidation current. The selective binding of cortisol within the imprinted cavities, leads to blocking of the PB electron transfer pathways and thus to a decreased PB oxidation current. The extent of such current diminution reflects the sweat cortisol concentration and can thus serve as the analytical signal (FIG. 18B). Such change in the current is also confirmed with cyclic voltammetry (CV) and electrochemical impedance spectroscopy (EIS). The incorporation of the built-in PB redox transducer within the imprinted polymer, and systematic optimization of the experimental parameters, have enabled fast (3.5 min) label-free CA cortisol sensing at a low potential of +0.1 V, as determined by CV.

The resulting MIP-based electrochemical sensing, along with the low-cost, scalable single-use screen-printed fingertip cortisol sensor and the compact hand-held instrumentation (FIG. 18C), offers convenient semi-continuous profiling of changing cortisol levels. To demonstrate the applicability of such MIP sensing platform, a stretchable epidermal patch (FIG. 18D) is also developed for tracking cortisol levels during physical activity. Utilizing formulized stretchable ink and adapting the “island-bridge” structure with skeleton layer reinforcement, the sensor demonstrated stable performance after repeated bending and stretching for on-body cortisol monitoring applications. The entire “touch-incubate-detect” protocol requires only 3.5 min, which is over ten times faster than common cortisol measurements, thus offers a distinct advantage for capturing sharply fluctuating cortisol levels in response to acute stimulations. Using such fast and simple cortisol testing platform, effortless and stress-free cortisol sensing can be realized toward tracking changing cortisol levels within a diurnal cycle (FIG. 18E). The variation of cortisol level during physical stimulations, which alters the endogenous cortisol level and is of importance to indicate injury, fatigue, dehydration/malnutrition, can also be captured using such sensing platform (FIG. 18F). The coupling of the simplicity and speed of the touch-based fingertip sweat analysis with a label-free MIP-based electronic detection thus enables dynamic stress-response profiling toward personalized healthcare and the management of personal stress and mental health.

Example Implementations for Optimization, and Characterization of the Touch-Based Cortisol Sensing

The new MIP detection relies on the selective binding of cortisol to the imprinted PPy membrane to impede the electron transfer process of the embedded PB redox probes. Non-imprinted PPy layers lack such recognition capability and exhibit no change in their signals in the presence of cortisol. FIG. 19A shows these processes at the MIP and non-imprinted polymer (NIP) surfaces, where the incoming cortisol molecules can occupy the MIP cavities to hinder the charge transfer of PB. The extent of such interaction, and overall sensitivity of the touch-based assay, are dependent on the cortisol concentration, and need to be carefully optimized. The interaction between cortisol and the MIP is studied first in 0.1 m phosphate buffer solution (PBS) as it establishes a stable, interference-free environment. The effect of the incubation time, which allows the binding of cortisol to the MIP layer, is tested from 5 s to 10 min using a 10×10⁻⁶ m cortisol solution along with amperometric detection at +0.1 V (FIG. 19B). The PB signaling current decreases rapidly upon increasing the incubation time, reflecting the increased cortisol interaction with the MIP, until reaching a near steady-state at 2 min.

Accordingly, a 2 min incubation time is used in all subsequent experiments. FIGS. 19C and 19D displays the detection of cortisol over a wide concentration range, from 1×10⁻⁹ m to 10×10⁻⁶ m in the PBS. A well-defined response is observed over this concentration range, leading to a logarithmic dependence of the current level with the cortisol concentration. A regression equation of I [nA]=(−38.9±0.4) log (C_{cortisol}) [×10⁻⁹ m]+(504.1±0.3), with the R2 of 0.9996 (n=3), is obtained. Notice the well-defined current response to the first 1×10⁻⁹ m cortisol concentration change. In contrast, a control experiment using the non-imprinted PPy electrode (FIG. 19G) shows a negligible response to similar additions of cortisol, reflecting the lack of cortisol binding cavities within the PPy layer. As the MIP sensors are intended for use as single-use disposable devices, the reproducibility of the sensors is crucial for obtaining reliable results. Five MIP cortisol sensors are thus fabricated and their response to 10×10⁻⁹ m cortisol is used for assessing the reproducibility of the synthesis and sensing of the MIP electrodes. As shown in FIG. 19E, the sensors exhibited highly reproducible cortisol responses with a relative standards deviation (RSD) of 1.42%. Selectivity is another important parameter essential for obtaining accurate stress profiling. The MIP sweat cortisol sensor offers selective recognition of the cortisol target, along with effective discrimination against a wide variety of common sweat constituents which can potentially interfere during the transduction step. FIG. 19F displays the current response of the sensor after incubating with physiologically relevant concentrations of different common interfering species, including glucose (Glu, 50×10⁻⁶ m), lactate (LA, 5 mm), urea (5 mm), ascorbic acid (AA, 50×10⁻⁶ m), acetaminophen (AP, 50×10⁻⁶ m), and uric acid (UA, 50×10⁻⁶ m), followed by the addition of 1×10⁻⁶ m cortisol. While no response is observed in the presence of the large excess of all these potentially interfering species, the MIP sensor displays a well-defined signal in the presence of cortisol, reflecting the highly specific MIP cortisol recognition.

After confirming the reproducibility, selectivity, and concentration dependence in the controlled PBS media, the MIP sensors are further characterized and evaluated in artificial sweat (AS) environment in the porous PVA hydrogel to simulate practical sweat sensing applications of the touch-based fingertip platform. As shown in FIG. 19H, the touch-based sensing is performed by collecting natural sweat from the fingertip by touching the hydrogel over a pre-selected time, followed by the incubation and amperometric detection at +0.1 V. Accordingly, the touching and incubation times are evaluated and optimized. A PVA hydrogel soaked in AS containing 1×10⁻⁶ m cortisol (1×1 cm², 50 mg) is used to simulate the interaction of cortisol in the hydrogel during the incubation process. The results, displayed in FIG. 19I, indicate that a 2 min incubation time, corresponding to a sweat volume of 300-30 000 nL (Notes S3, Supporting Information), is optimal for the touch-based sensing operation. The touching time is optimized by placing the subject's finger onto the hydrogel for variable time periods before the incubation step. As shown in FIG. 19J, the current steadily diminished upon increasing the touching time from 5 to 30 s and leveled off with longer touching times. Thus, the optimal conditions for the touch-based sensing are determined to be a 2-min incubation time and a touching time of 30 s, which are adopted for subsequent experiments. In agreement with the solution-phase experiment of FIG. 19G, no change in the current signal is observed using the NIP surface layer, confirming that the observed response is solely due to the specific MIP recognition of cortisol present in the fingertip natural sweat. To ensure that the measured signal reflects the interaction of the finger sweat cortisol with the MIP electrode, and not due to other mechanical factors (pressure, friction, etc.), the finger is wrapped with Saran plastic film prior to touching the hydrogel. As shown in FIG. 19L, no current response is observed using the wrapped finger, reflecting the lack of sweat transfer to the hydrogel. The quantitative aspects of the MIP fingertip sweat sensor rely on monitoring the decreased PB current in the presence of increasing cortisol concentrations. Using the hydrogel soaked in different cortisol concentrations in AS, a calibration plot is constructed over the 10×10⁻⁹ m to 1×10⁻⁶ m cortisol range. The resulting current response depended logarithmically on the cortisol concentration, following regression equation of I [nA]=(−60.3±4.2) log (C_{cortisol}) [×10⁻⁹ m]+(241.0±10.9) with R2 of 0.9853 (n=3) (FIGS. 19M and 19N). Itis worth noting that the calibrations in FIGS. 19D and 19N have shown good reproducibility in both PBS and AS (with the hydrogel environment) over the entire concentration range, given the similar error range shown in both plots. Calibration using the immunosensor is also performed in AS. Such calibration equation is used for all subsequent in-vivo experiments, considering the similarity of the AS medium to natural sweat from the fingertip. Note also the well-defined response to 10×10⁻⁹ m cortisol that reflects the remarkable sensitivity of the new fingertip-based MIP cortisol sensor. Such high sensitivity of the cortisol sensor corresponds to 38.8 nA log [nm]⁻¹ and 60.31 nA [nm]⁻¹ in PBS and AS, respectively. The signal-to-noise ratio (SNR) is SNR_{PBS}=116 and SNR_{AS}=25 while the limit of detection is 0.9 and 0.2×10⁻⁹ m using PBS and AS, respectively. The signal-to-noise ratio and the limit of detection of the cortisol sensor, as well as the response of the sensor to different pH, temperatures, and pressing pressures, have also been characterized; the sensor exhibited good stability using these different operational conditions.

Cortisol Monitoring and Validation in Circadian Cycles

The performance of the new cortisol sensor is first evaluated by monitoring the variations of endogenous cortisol levels during the diurnal cycles. Numerous studies have shown the correlation of cortisol levels with the circadian rhythm, where larger cortisol concentrations are present during the morning, decreasing during the day, and finally reaching lower levels in the evening (FIG. 20A). Dynamic tracking of such cortisol levels semi-continuously is of considerable importance for assessing the chronic stress level of individuals. Daily variations in the response of the touch-based sweat cortisol sensor are thus monitored and validated. The cortisol levels of 5 patients are measured at 7 a.m. and 5 p.m. on the same day using fingertip sweat, along with validation via immunoassay of pilocarpine-stimulated sweat samples. The optimized touching and incubation times are used, and the cortisol signal is acquired with a portable potentiostat, with the entire assay requiring 3.5 min (FIG. 20B). Considerable differences, ranging from 86 to 200×10⁻⁹ m cortisol, are observed for all subjects using the finger-based cortisol sensor. FIG. 20C displays the amperometric cortisol response of three patients for this morning/evening experiment. The background signal is measured with only the sweat collector gel on the sensor surface, followed by the sweat cortisol measurements in the morning (red curve) and the evening (blue curve); a new sensor is used for recording each response. The immunosensor-based validation of the fingertip MIP sensor involved stimulated sweat using 10 min pilocarpine-based iontophoretic (IP) extraction on the user's forearm, followed by 20 min collection with a PDMS microfluidic epidermal device, placed on the sweat stimulated area (FIG. S13, Supporting Information). FIG. 20D displays the correlation between the MIP-fingertip sweat sensor and the corresponding immunoassays (solid and hatched bars, respectively). These data indicate a strong correlation (with Pearson's r=0.96) between the cortisol sweat concentrations estimated by the fingertip MIP sensor and by the corresponding immunoassay. Following the sweat validation testing, the new sweat finger sensor is successfully used to monitor the morning and evening cortisol levels of additional patients, displaying clear differences in the concentrations during these periods (FIG. 20E). The fast and convenient use of the finger cortisol sensor is demonstrated by monitoring the cortisol levels of several patients throughout the day (FIG. 20F). For this, the sensor response is recorded every 2 hours, over 12 hours, from 7 a.m. to 7 p.m. A gradual decrease in the sweat cortisol level is observed for all patients between the morning to the evening measurements. Further, exercising during the day increases momentary the circulating cortisol levels (FIG. 20F (a), (b)). Two patients are thus asked to include an exercising routine (involving 30 min indoor biking) in their daily activities during such semi-continuous cortisol monitoring. Sweat cortisol levels increased right after exercising, decreasing to the endogenous levels within 2 h. It is worth noting that the sweat-inducing protocol is used only at rest once natural perspiration occurs during exercise. Attempting sweat stimulation during physical activity would result in a mixture of chemically and exercise-induced sweat. Therefore, in order to assess and validate the exercise stress stimuli, the sweat induced protocol is used right after the exercise routine (FIG. 20F (b), (c)). This experiment clearly shows the need for having effortless sweat cortisol sensing, since exercising inducing cortisol can affect the endogenous cortisol levels. Besides the substantially simpler operation, the use of the fingertip sweat MIP sensor has distinct speed advantages-compared to the induced-sweat immunoassays-which is extremely important for capturing instantaneous and sharp variations in cortisol levels.

Monitoring Cortisol Response to Acute Physical Stimuli

The ability to monitor in near real-time fast variations in sweat cortisol is demonstrated by using a stress-inducing cold-pressor test (CPT) (FIGS. 21A-21F). The CPT, performed by immersing the subject's hand into an ice water container for 3 min, is a common and well-validated laboratory stressor that directly activates the hypothalamus-pituitary-adrenal axis to release cortisol. Thus, to monitor the fluctuation of the cortisol level due to the induced stress from the CPT, participants are asked to immerse their non-dominant hand in an ice water bath for 3 min (FIG. 21B), followed by measuring their cortisol level using the touch-based cortisol sensor every 5 min up to 20 min. Each experiment is conducted at 5 p.m. using the same protocol, involving a 30 s touching time and 2 min of incubation time. FIGS. 21C-21E displays the dynamic cortisol profile and the current signals at 0 min (blank solid) and 10 min (blue solid) after removing the hand from ice water for three different subjects, indicating that the sharp fluctuations of the cortisol level can be rapidly captured from the fingertip-collected sweat. In all three cases, the maximum concentration of cortisol is reached after 10 min and almost recovered after 20 min. The touch-based fingertip cortisol sensor offers a distinct advantage for tracking such rapid CPT-induced fluctuations of the cortisol level compared to other cortisol sensing mechanisms that require lengthy biofluid extraction or complex sensing procedures. It is worth noting that the pilocarpine-induced sweat collection procedure, which takes over 20 min (IP time+collection time), is not used for validation, as the time lag of the corresponding collected sweat samples cannot temporally resolve the rapid CPT-induced variations. Moreover, additional healthy patients (n=7) measured their cortisol concentration before and after 10 min of CPT, as shown in FIG. 21F. All participants displayed significantly higher cortisol levels following the CPT stimulation compared to the level before the CPT. Overall, the data of FIGS. 21A-21F clearly show the ability to detect short duration spikes in circulating cortisol induced by external stimulus. The commonly used IP sweat stimulation technique is not able to detect such fast variations in sweat cortisol owing to its corresponding long time lag. This is indicated from the corresponding cortisol concentrations in stimulated sweat after cold water stress.

Stretchable Epidermal Cortisol Sensor Patch for Sweat Sensing

Beyond its use in finger-based natural sweat sensing, the reliable and highly selective IP-based cortisol sensing mechanism can be readily adapted onto various wearable form-factors for different sensing applications. As is demonstrated from FIGS. 21A-20F, an acute physical stimulus, such as exercising, can effectively increase cortisol levels in individuals. The finger-based cortisol sensor requires the subject to steadily press the sensor for sweat collection. However, as sweat can be rapidly accumulated during the exercise sessions, the continuous sensing of cortisol from the exercise-induced sweat is possible, with no additional collection step, flexible IP-based epidermal patch is thus fabricated using soft, stretchable substrate and stretchable, screen-printable inks, including serpentine structures and skeleton-layer shape confinement to limit the deformation on the MIP sensing region (FIG. 18D). In particular, a stretchable silver ink is printed as the interconnecting “bridges” whereas the dielectric skeleton layer is printed below the electrodes and contact points as islands to ensure no strain is applied to the electrodes, hence establishing a stable “island-bridge” configuration. A soft bilayer soft substrate with Ecoflex and polyurethane is fabricated to ensure the conformal contact of the sensor to curved body surfaces while ensuring the substrate bonding with the inks. Previous studies have demonstrated the advantageous combination of the soft substrate and the “island-bridge” configuration that ensures the mechanical durability of the electrodes against rigorous movements. As the structural engineering and selection of the substrate material do not affect the electrochemical performance of the sensor, the epidermal patch displays a similar analytical performance to that of the finger-based cortisol sensor, with the addition of the superior mechanical durability. Building on the attractive performance of the flexible cortisol MIP sensor in AS medium, on-body evaluation of the epidermal patch is thus carried out on three patients at 7 a.m. and 5 p.m. on the same day, where high and low cortisol levels are expected based on the established circadian rhythm. FIG. 22A shows the experimental protocol used for on-body trials; this involved application of the epidermal patch to patients' forearms after 15 min of indoor cycling activity and measuring the amperometric response following the 2-min incubation time, while the subjects are still in motion.

The mechanical stability of the sensor is tested first using CV, aiming to assess the electrochemical behavior of the new MIP sensor during severe mechanical deformations. CV in PBS solution containing 1.0 mm [Fe(CN)₆]^{3-/4-} redox probe is thus used to evaluate the effects of bending and stretching deformations on the electrochemical performance. The sensors are both bent to 90° and stretched to 20% strain repeatedly, with their CVs recorded every 10 cycles to compare the sensor performance throughout the deformation cycles. As a single-use sensor, 50 cycles of deformations are considered significant, and the deformation is carried out over 60 cycles to ensure the sensors' stability. As shown in FIG. 22C, the wearable sensor is able to maintain its stable performance within bending 60 cycles, yielding highly reproducible CV and peak currents throughout this bending experiment (FIG. 22C (a), (b)). Similarly, no visible change in the voltammograms, including the corresponding peak currents, is also observed during these 50 stretching deformation cycles (FIG. 22C (c), (d)).

After confirming the mechanical and electrical resiliencies of such sensors, their application in on-body tests is carried out. FIG. 22D displays the current signals obtained for the three patients, showing a very similar trend for all participants with the lower current signals (i.e., higher cortisol levels) in the morning compared to the results in the afternoon. These clear signals, along with the low noise levels and background signals, demonstrate the reliability of the sweat uptake and the sealed electrical connections. Comparing these current signals with the calibration plot of FIG. 19C resulted in morning cortisol levels of 324, 405, and 363×10⁻⁹ m, and afternoon concentrations of 63, 78, and 37×10⁻⁹ m, respectively. Immunoassay measurements are also employed to validate the cortisol concentrationusing sweat samples collected from the subject at the same time. These data show a good agreement between the on-body results of the wearable patch sensor and the immunoassay test. Thus, the exercising sweat-sensing of such epidermal sensor indicates great potential toward rapid sensing of cortisol level in real-time even during highly dynamic physical movements.

The disclosed technology can be implemented in some embodiments to provide a simple, label-free, effort-less, low-cost detection platform for the rapid sensing of cortisol concentrations in natural fingertip sweat using an electrochemically synthesized MIP membrane with a built-in PB redox probes. Using the developed porous PVA hydrogel, the cortisol in the passive natural sweat, accumulated on one's fingertips, can be easily sampled without the need for stress-inducing exercise nor lengthy extractions. Upon short touching and incubation time, the synthesized PPy-based cortisol MIP allows the label-free, rapid, and direct measurement of cortisol concentrations from the decreased current response of the PB redox probe embedded in the polymeric network. Such fast fingertip assay eliminates time delays characteristic of common cortisol assays, thus enabling near real-time monitoring of rapidly changing cortisol concentrations. Using this touch-based fingertip sweat sensing platform, the long-term cortisol level fluctuations of multiple subjects within the circadian cycle can be monitored and the measurements can be validated using an established immunoassay involving IP-stimulated sweat. By avoiding the need for any stress-inducing activity (e.g., exercise) for sweat sampling, the new sweat-based MIP-based method offers accurate cortisol measurements in a stressless fashion. In addition, the rapid and effortless sampling of fingertip sweat allows the capturing of cortisol level fluctuation during an acute stimulation event, such as CPT. As a platform detection technology, the MIP-based sensing is also adapted to a form factor of a stretchable and wearable patch for the direct sensing of sweat cortisol levels during exercising, eliminating the sampling time and further expedited the sensing speed. The scope of such MIP-based fingertip can be expanded for the detection of other hormones and biomarkers. Further improvement could be achieved by parallel use of pH, temperature, and flow-rate sensors to account for the fluctuations in the sweat and body parameters. Overall, the new MIP-based fingertip cortisol sensing, leveraging numerous material innovations, offers a reliable and practical approach for rapid and stress-free stress monitoring, and it can be used for managing personal stress or mental health, guiding future research in this area, thus having a profound implication to the fields of wearable sensors, mobile health, and personalized healthcare.

In some implementations, a finger-based sensor electrode can be fabricated as follows. The electrodes for the finger-based cortisol sensor are fabricated by screen-printing using a semi-automatic MMP-SPM printer and custom stainless-steel stencils developed using AutoCAD software, with dimensions of 12 in.×12 in. and 75 μm thickness. The electrodes are printed layer-by-layer. First, the silver/silver chloride ink is printed onto a poly (ethylene terephthalate) (PET) substrate as the interconnection and reference electrode, followed by printing a layer of carbon ink as the working and counter electrodes. Each layer is cured at 80° C. for 10 min in the oven. Lastly, a polymer insulator composed of SEBS, dissolved in toluene (35 wt %), is printed onto the electrodes to define the working electrode area and insulate the exposed interconnections.

In some implementations, a stretchable sensor patch can be fabricated as follows. A stretchable substrate is fabricated by printing a thin layer of Ecoflex onto the adhesive side of a Perme-Roll Lite film. A stretchable silver ink is formulated by mixing a SEBS resin (31.5 wt % in toluene) with silver flakes in a planetary mixer at 1800 rotations per minute (RPM) for 5 min. A stretchable carbon ink is formulated by mixing the same SEBS resin, toluene, graphite, and Super-P in a 12:3:8.5:1.5 weight ratio at 2250 RPM for 10 min. The dielectric ink is first printed onto the Perme-Roll side of the stretchable substrate as the skeleton layer and cured in the oven at 80° C. for 10 min.

The stretchable silver is then printed as the interconnect and the stretchable carbon as the working and counter electrodes. Both inks are cured in the oven at 80° C. for 5 min. The Ag/AgCl ink is printed as the reference electrode and is cured in the oven at 800 for 10 min. Lastly, the SEBS resin is printed to define the electrode area and insulate the interconnections and cured in the oven at 80° C. for 10 min.

In some implementations, a molecularly imprinted polymer can be synthesized as follows. The screen-printed electrodes are cleaned with CV over the potential range of −1.5 to +1.5V in a (0.5 m H₂SO₄) solution for 10 cycles (using a scan rate of 50 mV s⁻¹). Then, the sensors are washed twice with deionized water and left to dry at room temperature. The fabrication procedure for the MIP film is performed via electropolymerization using CV at −0.2 to +0.9 V potential range with a scan rate of 50 mV s⁻¹ for 10 cycles in PBS solution (pH=7.4) containing 0.02 mol L⁻¹ pyrrole, 5 mmol L⁻¹ FeCl₃, 5 mmol L⁻¹ K₃[Fe(CN)₆], 6 mmol L⁻¹ cortisol, and 0.1 mol L⁻¹ HCl. After the electropolymerization process, the electrode is washed with deionized water twice to remove the remaining compounds. The embedded cortisol molecules are then extracted from the PPy-PB matrix through over-oxidation of PPy-PB by CV at the potential range from −0.2 to +0.8 V for 20 cycles (at 50 mV s⁻¹) in PBS to produce the complementary cavities.

For the preparation of NIP, the same preparation method is applied as MIP, excluding the cortisol molecule as a template during the polymerization step. Although the polymerized layer did not contain the template, still the PPy over-oxidation step is performed to make sure the other experimental condition is the same as the MIP sensors. Finally, the prepared NIP based electrode is washed twice with deionized water and dried at room temperature until use.

In some implementations, the porous PVA hydrogel can be fabricated as follows. The fabrication of the porousPVA hydrogel is based on previous studies with modifications. First, solution of the PVA (MW≈89 000) dissolved in water in a 1:10 weight ratio and KOH dissolved in water in a 1:5 weight ratio is prepared. Then, 14 g of KOH solution is added dropwise to 10 g of PVA solution with stirring, followed by dissolving 2.6 g of sucrose into the mixture to form the hydrogel precursor. 15 g of the precursor is then poured into a Petri dish (diameter≈9 cm) and left in a vacuum desiccator to remove excess water and allow cross-linking until only ⅓ of the weight of the precursor is left. The crosslinked gel is then soaked in 0.1 m PBS buffer to remove the sucrose template and the excess KOH until the gel is in neutral pH. The gel could then be cut into desired sizes and shapes and stored in PBS or AS for subsequent use. The resulted hydrogel had a uniform thickness of 400 m.

In some implementations, the artificial sweat can be prepared as follows. The AS is prepared in PBS 0.1 m, pH 7.4 by adding the major sweat constituents: NaCl (85×10⁻³ m), KCL (13×10⁻³ m), lactate (17×10⁻³ m), and urea (16×10⁻³ m). A buffered solution is used in the AS formulation to prevent signal fluctuations due to changes in the sweat pH. For the fingertip sweat cortisol testing, the PVA gel is loaded with 40 μL of AS prior to touching the sensor.

In vitro sensors can include the following features. All electrochemical performances of MIP based sensor are evaluated in a 0.1 m PBS (pH 7.4), AS, and PVA gel with each solution. The CAs are conducted under the potential at +0.1 V (vs Ag/AgCl) for 60 s. The calibration plots for MIP and NIP based sensing platform are obtained by measuring the concentration range of cortisol from 1×10⁻⁹ m to 10×10⁻⁶ m in PBS or 10×10⁻⁹ m to 1×10⁻⁶ m in AS. The selectivity is examined by measuring the response to differentrelevant interference species such as 50×10⁻⁶ m glucose, 5×10⁻³ m lactate, 5×10⁻³ m urea, 50×10⁻⁶ m ascorbic acid, 50×10⁻⁶ m acetaminophen, and 50×10⁻⁶ m uric acid, respectively, and further measured the response to the addition of 1×10⁻⁶ m cortisol in the presence of all the interferences. The reproducibility is evaluated by measuring the response to 10×10⁻⁹ m cortisol at five differentMIP-based sensors in PBS solution. The mechanical resilience of the MIP based wearable sensor is evaluated by transferring it to transparent plastic substrate to mimic the flexible properties of the skin and measuring the CV response in 1.0×10⁻³ m [Fe(CN)₆]^{3-/4-} solution after repeated 90° bending and 25% stretching. The CV response is recorded every 10 times of the repeated stretching and bending up to 60 times, respectively.

For the circadian rhythm measurements (7 a.m. and 5 p.m.), each healthy user washed their hands before the experiment and touched the PVA gel for 30 s. After 2 min of incubation time, the CA is recorded at an applying potential of +0.1 V for 60 s and the concentration is calculated based on the previous calibration plot obtained from the in vitro experiment. Meanwhile, sweat is induced using IP and collected to validate the concentration of cortisol with immunosensor. The continuous cortisol monitoring is conducted with three subjects (one without any exercise and two with exercise at 12:30p.m. and 4:30p.m. for 30 min) recording signal from 7 a.m. to 7 p.m. at every 2 h. A fresh sensor is used for each measurement.

Three patients participated in the cold pressor test (5 p.m.) by immersing their left hand in a container with ice water for 3 min. After 3 min, participants removed their hands from the ice water and measured the cortisol levels every 5 min interval to track the fluctuation of cortisol using the right hand. Moreover, seven healthy patients contributed to the cold pressor test by gauging before and after 10 min of dipping their hands. The procedure of the measurement is conducted by CA, with sequentially washing hand, touching for 30 s, and incubate for 2 min. A fresh sensor is used for each measurement.

High Energy Return on Investment Harvesting from Fingertip Passive Perspiration

Self-powered wearable systems relying on bioenergy harvesters commonly require excessive energy inputs from the human body, and are highly inefficient when accounting for overall energy expenses. A harvester independent from external environment for sedentary state has yet to be developed. The disclosed technology can be implemented in some embodiments to provide a touch-based lactate biofuel cell that leverages the high passive perspiration rate of fingertips for bioenergy harvesting. Powered by finger contact, such harvesting process can continuously collect hundreds of mJ of energy during sleep without active input, representing the most efficient approach compared to any reported on-body bioenergy harvesters. To maximize the energy harvesting, complementary piezoelectric generators are integrated under the biofuel cell to further scavenge mechanical energy from the finger presses. The harvesters can rapidly and efficiently power sensors and electrochromic displays to enable independent self-powered sensing. The passive perspiration-based harvester establishes a practical, high energy return-on-investment example for future self-sustainable electronic systems.

Wearable electronics have witnessed a tremendous growth over the past decade. Current wearable electronics are predominately powered by miniaturized electrochemical energy storage devices (e.g., batteries, supercapacitors), with limited energy and power density that cannot power the electronics over extended operational time. To address this challenge, researchers have focused on reducing the energy consumption while introducing energy harvesters to offer extended system runtime. Self-powered sensors that autonomously generate signals can reduce the system power consumption but cannot provide sufficient energy to the electronics for the actual measurement or data transmission. Recent progress in energy harvesters has enabled self-sustainable systems that continuously harvest energy from sunlight, movements, temperature gradients, or biofuels to power the sensors and electronics intermittently or continuously. However, harvesters based on an inconsistent external environment cannot supply energy on command, while mechanical and biochemical energy harvesters require vigorous movement and with high mechanical energy investment, thus are highly inefficient, inconvenient, and lack practicality. An energy harvester relying on a passive constant input from the human body, not relying on from irregular external environment nor movements and exercises, is therefore considered a holy grail of energy harvesting devices.

Among all aforementioned energy harvesters, lactate-based biofuel cells (BFCs) have shown considerable promise as self-powered sensors and bioenergy harvesters for powering electronics. Relying on the high lactate concentration in human sweat, epidermal BFCs can readily generate energy using a lactate oxidase (LOx) bioanode complemented by the oxygen reduction reaction (ORR) on the cathode. However, despite of their great potential for powering wearable electronic devices, the ability to exploit the rich sweat bioenergy has been hindered by the inherent inaccessibility of natural sweat. While sweat is autonomously generated from the human body in most of the epidermal spaces, its flow rate is extremely low for realizing efficient bioenergy harvesting. Thus, wearable BFCs commonly require vigorous and extended exercise before a sizable amount of sweat can accumulate onto the bioelectrodes for power generation. While epidermal BFCs with high power density have been reported, the operation of such BFC-powered systems requires massive energy input towards continuous sweat generation, resulting in extremely low conversion efficiency (<1%) when accounting for the mechanical energy input (Table 1 below). Alternative approaches for accessing sweat biofuels without intensive exercise are thus urgently needed for routine and practical applications of BFCs in wearable systems.

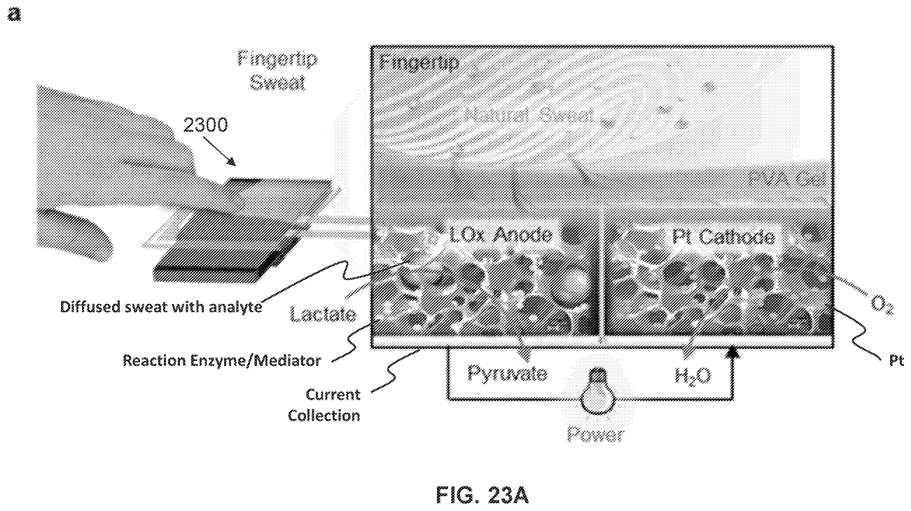

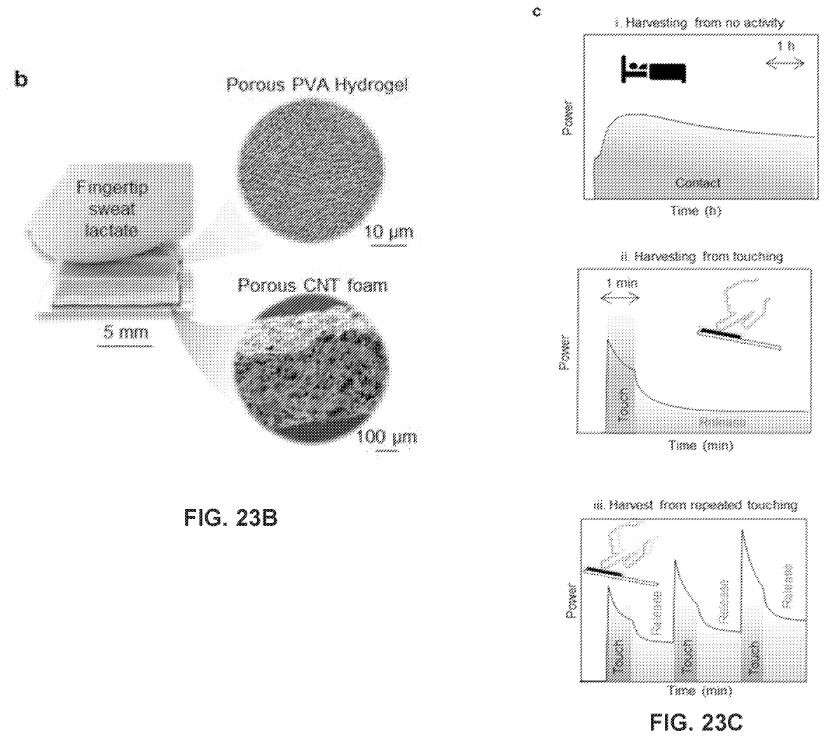

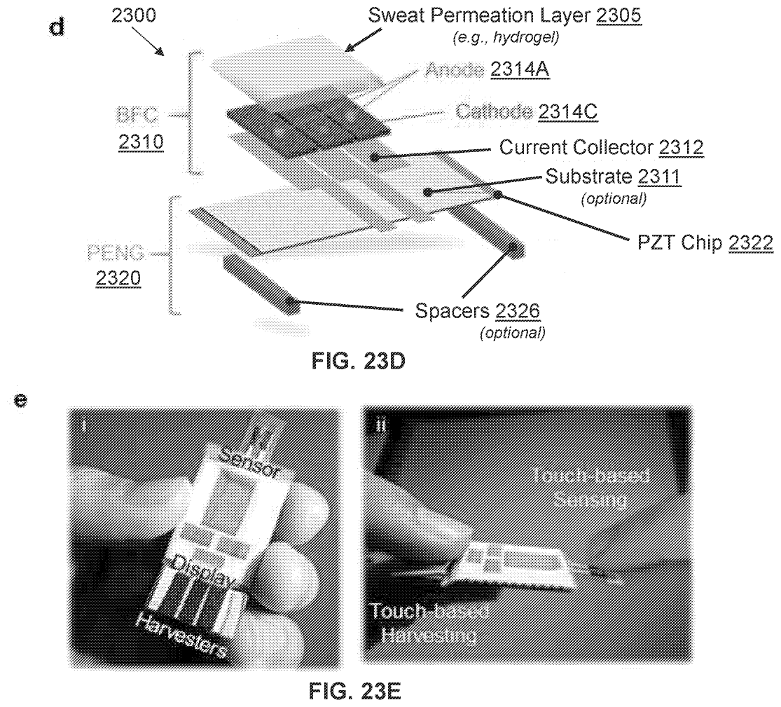

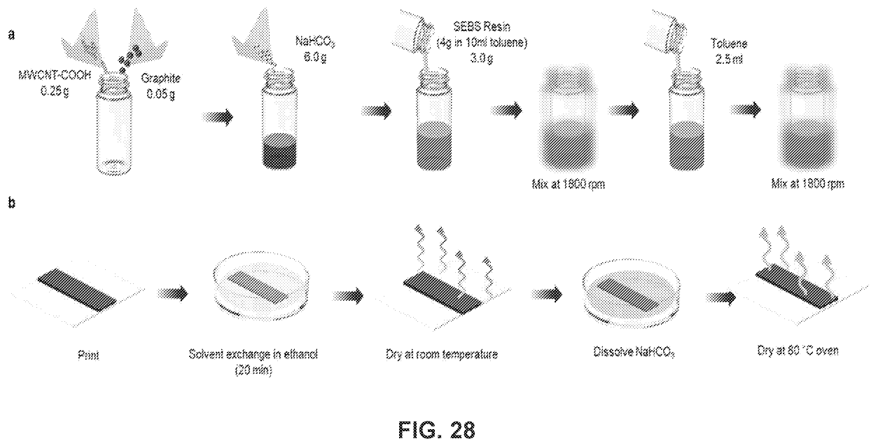

The disclosed technology can be implemented in some embodiments to provide a high energy return-on-investment (EROI) harvesting device powered by natural, passive fingertip sweat and does not require mechanical input to instantly generate power. Optimized for collecting the natural perspiration from a finger, the disclosed technology can be implemented in some embodiments to provide a flexible, porous, water-wicking 3-dimensional (3D) carbon nanotube (CNT) foam (e.g., some examples shown in FIGS. 28-30 and 32) to be implemented as the BFC electrodes (e.g., anode and cathode electrodes), in which the 3D CNT foam BFC electrodes can be decorated with LOx and nanoporous Pt on the anodic and cathodic sites for lactate oxidation and oxygen reduction, respectively, for bioelectrocatalytic powergeneration (FIG. 23A).

FIGS. 23A-23D show diagrams and data plots depicting example embodiments of and implementations for operation of a touch-based biofuel cell (BFC) and bioenergy harvesting system in accordance with the present technology. FIG. 23A shows schematic illustration of an example analyte-harvesting BFC device designed for BFC-harvesting of a lactate biofuel from the natural finger sweat, which includes a LOx-modified anode and Pt-modified cathode formed from the 3D CNT foam and disposed under an example embodiment of a sweat permeation layer (e.g., a templated porous PVA hydrogel) and above an example lead zirconate titanate (PZT) chip. FIG. 23B shows optical and SEM images of the templated porous PVA hydrogel and CNT foam. FIG. 23C shows illustration of three operating conditions of the BFC, harvesting energy from (i) passive continuous contact, (ii) active pressing, and (iii) repeated active pressing. FIG. 23D shows exploded view of the example integrated BFC-piezoelectric energy generation analyte-harvester device, configured to generate chemical and mechanical energy harvested from constituents in natural sweat transferred across the sweat permeation layer from the press of fingers upon the device. FIG. 23E shows photo images of (i) self-powered sensing system with integrated harvesters, sensor, and ECD, and (ii) device sensing sweat composition from the natural finger sweat.

Referring to FIGS. 23A and 23D, an example of the touch-based biofuel cell and bioenergy harvesting (BFC-BH) system 2300 based on some embodiments of the disclosed technology. The example BFC-BH system 2300 can include a biofuel cell (BFC) assembly 2310 integrated with a piezoelectric energy generation (PENG) assembly 2320 and an example embodiment of the sweat permeation layer 115, shown as sweat permeation layer 2305 in FIG. 23D. The BFC assembly includes two or more electrodes 2314, comprising an anode electrode 2314A and a cathode electrode 2314C, which are coupled to a current collector 2312, which may include two or more electrically-conductive material structures (e.g., configured to be planar or have other geometries) to electrically couple at least one electrically-conductive material structures to the anode and cathode, respectively. In some embodiments, the BFC assembly 2310 may include a substrate 2311, upon which the current collector 2312 is disposed, which is coupled to the electrodes 2314. The sweat permeation layer 2305 is configured to be coupled to the plurality of electrodes 2314, and may include a flexible, porous hydrogel material, e.g., such as a PVA gel embodiment (described herein). In some implementations, the plurality of electrodes 2314 may include a flexible, porous, water-wicking 3-dimensional (3D) carbon nanotube (CNT) foam, also shown in FIGS. 28-30 and 32. In some implementations, the anode 2314A may be modified by substances configured to facilitate a reaction with the target analyte to create a detectable electrical signal, such as enzymes and/or mediators, which in the example shown in FIG. 23 A includes LOx; and the cathode 2314C may include nanoporous Pt (e.g., Pt particles or Pt-coated particles embedded into the example 3D CNT foam), which together facilitate for lactate oxidation and oxygen reduction, respectively, for bioelectrocatalytic power generation. In some implementations, the sweat permeation layer 2305 may include a porous polyvinyl alcohol (PVA) hydrogel, e.g., which is capable to eliminate the Laplace pressure of sweat droplets for facilitating continuous sweat transfer from the fingertip to the BFC electrodes, while retaining the fuel toward continuous harvesting. The PENG assembly 2320 includes a piezoelectric substrate or chip 2322 able to undergo non-destructive mechanical deformation upon depression of the BFC-BH system 2300 by user's fingertip, in which electrical energy is generated from the mechanical deformation. In some embodiments, the piezoelectric chip 2322 includes PZT. The piezoelectric chip 2322 is located directly below the BFC energy harvester assembly, e.g., under the optional substrate 2311, and is activated upon a slight finger press. The power generated on the piezoelectric material (e.g., PZT) increases upon raising the pressing force, frequency, and deformation. In some embodiments, the PENG assembly 2320 optionally includes two or more spacers 2326 disposed under the piezoelectric chip 2322 and above an optional base substrate (not shown). In implementations, for example, the spacers 2326 can be used to control and effect the power generated by the piezoelectric material 2322, e.g., based on controlling the thickness of the spacers 2326.

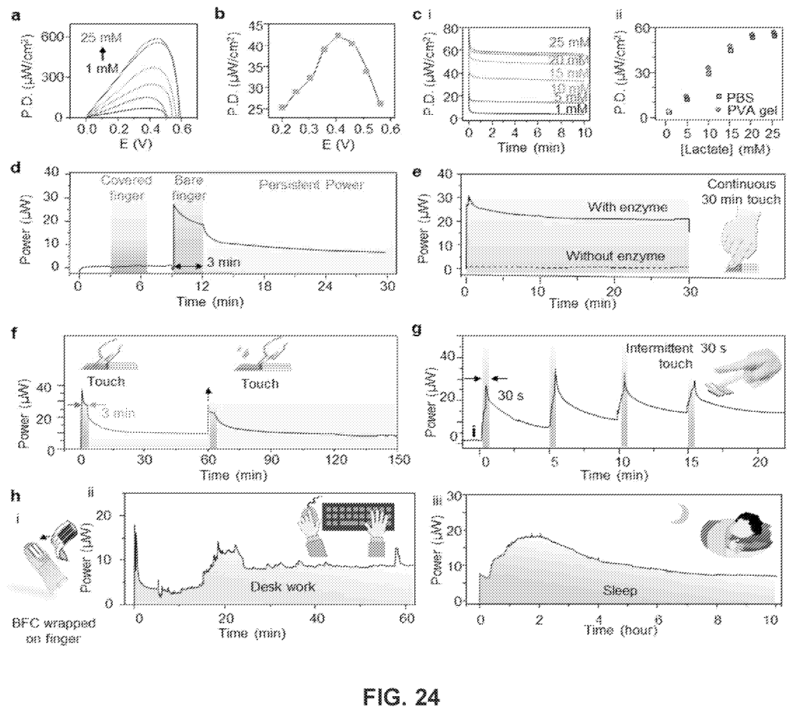

FIG. 24 shows data from an example in-vitro and in-vivo characterization implementation of the example touch-based BFC and bioenergy harvesting system: (a) the areal power density of the BFC at different lactate concentration (1, 5, 10, 15, 20, 25 mM) characterized using LSV at 5 mV/s; (b) the areal power density of the BFC at different potentials, characterized via 10-min CA; (c) power of the BFC (i) at 0.4 V in PBS with different lactate concentrations and (ii) the power calibration plot of the BFC with different lactate concentrations in PBS and with PVA gel; (d) comparison of the power of the BFC touched by a covered finger for 3 min and by a bare finger for 3 min; (e) power profile of the BFC during 30-min continuous pressing, using anode decorated with and without LOx enzyme. (f), Power profile of refuelling by pressing the BFC for 3 min after 1 h of resting. (g), Power profile of the BFC during repeated 30 s pressing every 5 min. FIG. 2d-g, pressing pressure, 50 kPa; CA voltage, 0.4 V. h, (i) illustration for the BFC attached to the finger for long-term continuous energy harvesting. (ii) Power profile during a 1-h normal desk work involving intermittent BFC pressing. (iii) Power profile of the BFC passively harvesting bioenergy overnight (10 h sleep) from a finger.

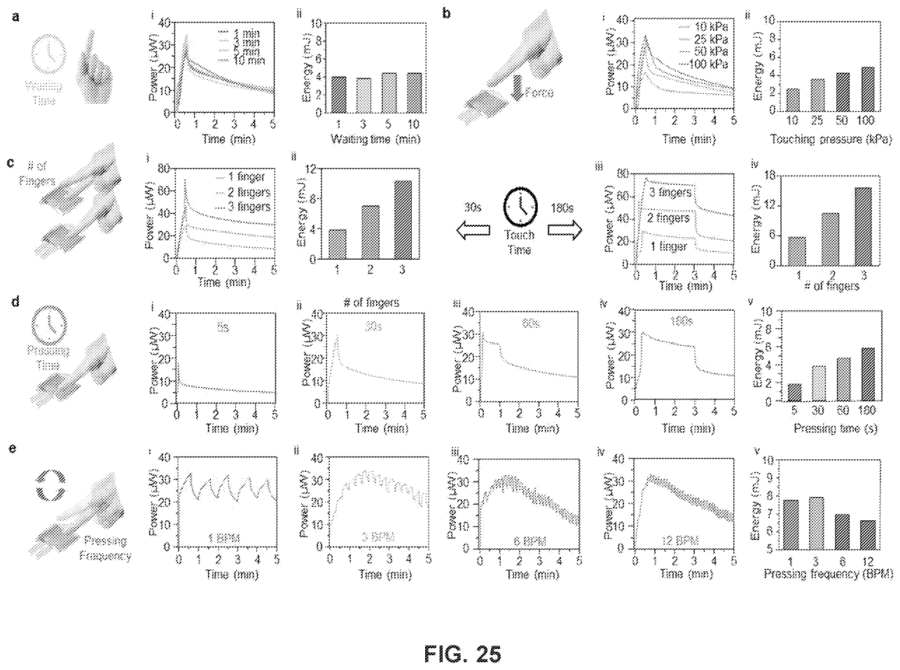

FIG. 25 shows data from an example optimization implementation for BFC usage patterns of the example touch-based BFC and bioenergy harvesting system: (a) the power generation profile and energy harvested within 5 min of the BFC touched with the pressing pressure of 50 kPa by a bare finger that has been cleaned and waited for various time periods before touching once for 30 s; (b) the power generation profile and energy harvested within 5 min of the BFC touched by a bare finger once for 30 s with different pressing weights; (c) the power generation profile and energy harvested during 5 min of the BFC touched with the pressing weight of 50 kPa by 1-3 fingers paired with corresponding number of BFCs for (i) 30 s and (ii) 3 min; (d) the power generation profile and energy harvested within 5 min of the BFC touched with the pressing weight of 50 kPa by a bare finger once for different time periods (5-180 s; i-iv); and (e) the power generation profile and energy harvested during 5 min using different pressing frequencies with the pressing pressure of 50 kPa by one finger.

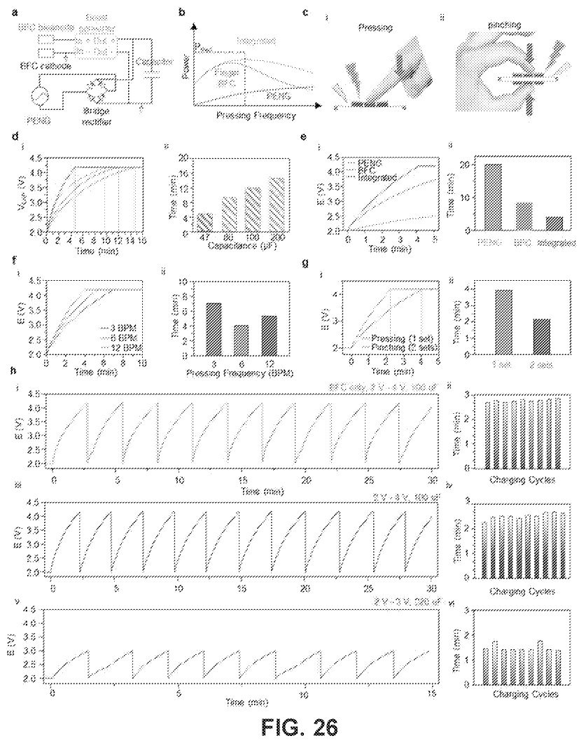

FIG. 26 shows data from an example performance implementation of the touch-basedBFC and the integrated harvesting system: (a) system diagram of the integrated BFC-PZT touch energy harvesting system, including an energy boost and regulation circuit; (b) illustration of finding the optimal energy harvesting operation setup; (c) the two modes of operation based on (i) pressing with 1 finger with 1 set of integrated harvesters and (ii) pinching with 2 fingers and 2 sets of integrated harvesters in a sandwich configuration; (d) One BFC harvester pressed with 6 BPM frequency (i) charging a capacitor with different capacitance and (ii) its corresponding charging time; (e) (i) The charging of a 100 μF capacitor using only one PZT harvester, one BFC harvester, and one integrated harvester pressing at 6 PBM frequency, and (ii) their corresponding charging time; (f) (i) The charging of a 100 μF capacitor using one integrated harvester pressing at different frequencies, and (ii) their corresponding charging time; (g) (i) The charging of a 100 μF capacitor using only one and two sets of integrated harvesters pressing at 6 PBM frequency, and (ii) their corresponding charging time; (h) (i) The charging of a 100 μF capacitor from 2 V to 4 V (i) using only two BFC harvesters pressing at 6 PBM frequency, and (ii) their corresponding charging time, and (iii) using two integrated BFC-PZT harvesters pressing at 6 BPM frequency, and (iv) their corresponding charging time, and (v) the charging of a 220 μF capacitor from 2 V to 3 V using two integrated BFC-PZT harvesters pressing at 6 BPM frequency and (vi) their corresponding charging time.

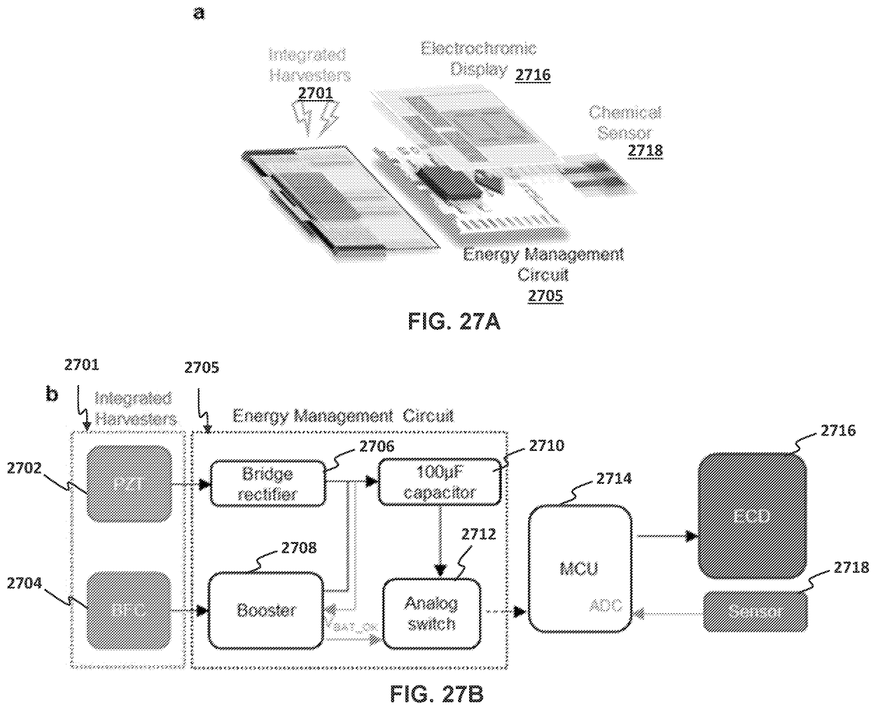

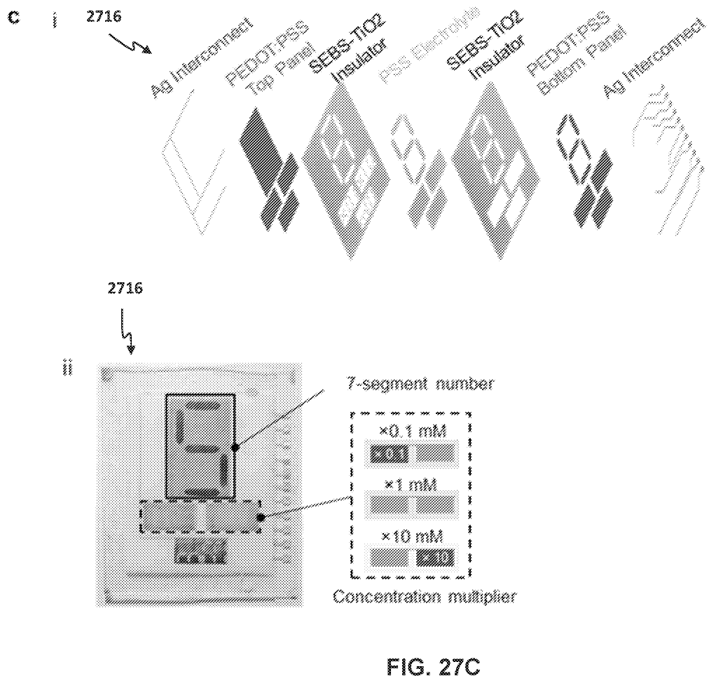

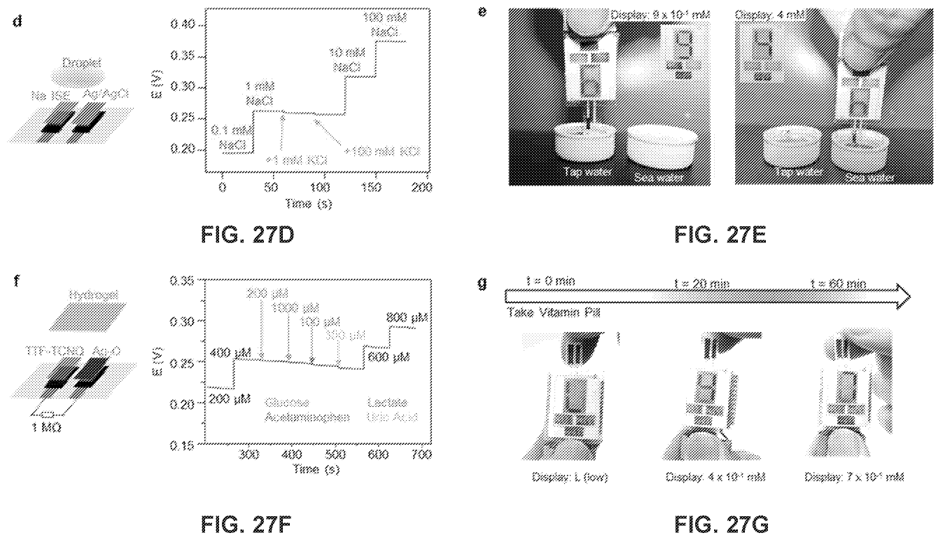

FIGS. 27A-27G show diagrams and data plots depicting example embodiments of and implementations for operation of self-powered sensor-display system in accordance with the present technology. FIG. 27A shows an exploded view of the device schematics which includes two pairs of BFC-PZT harvesters, 2-electrode sensor, ECD panel, and related MCU and power management circuit. FIG. 27B shows an example system diagram of the self-powered system. FIG. 27C shows an example of low-power ECD in (i) exploded view schematics and (ii) illustration of the readings on the display panel. FIG. 27D shows (i) illustration of the 2-electrode ion-selective sodium sensor, and (ii) the calibration and selectivity of the sodium sensor. FIG. 27E shows photos of the self-powered sensing system, detecting sodium concentration in tap water and in 1:100 diluted sea water. FIG. 27F shows (i) illustration of the 2-electrode vitamin-C sensor, and (ii) the calibration and selectivity of the vitamin-C sensor. FIG. 27G shows an example of time scale of the Vitamin C testing after taking a Vitamin pill (top) and corresponding photo images of the ECD reading at different time points after taking the pill tested using the self-powered sensing system.

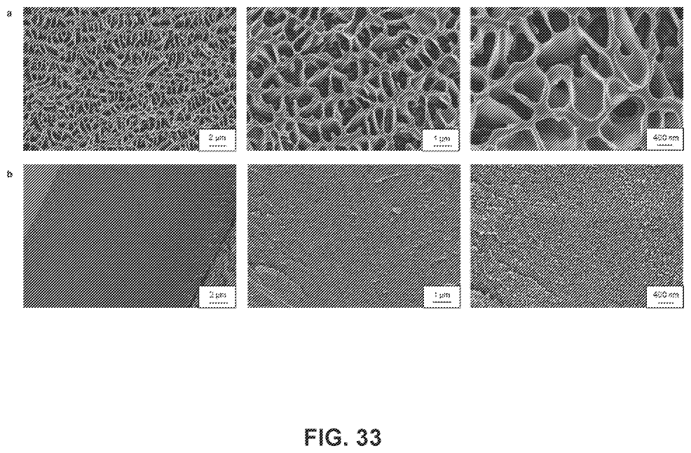

Unlike other body locations, the sweat rate on the fingertip is considerably high (80-160 gh⁻¹). Recent reports demonstrated the advantages of such fingertip natural perspiration for sweat analysis compared to common sweat stimulation methods (such as exercise, iontophoresis, or heats). Such efficient fingertip sweat generation is extremely attractive for powering BFCs without the need for any sweat-inducing exercise. A porous polyvinyl alcohol (PVA) hydrogel is further employed to eliminate the Laplace pressure of sweat droplets for facilitating continuous sweat transfer from the fingertip to the BFC electrodes, while retaining the fuel toward continuous harvesting (FIG. 23B, FIG. 33). The finger contact-based BFC can harvest continuously hundreds mJ of energy per cm² over 10 h of sleep without any mechanical input or harvests over 30 mJ energy per hour from a single press of a finger that consumes merely 0.5 mJ mechanical energy, resulting in a 6000% high EROI; repeated touching results in refuelling and enhanced convection, and can further boost the power to harvest more energy over a shorter time period (FIG. 23C). Implementing the microgrid design concept for self-powered electronic systems, this contact-based BFC has been combined with a PZT piezoelectric generator to further increase the harvesting efficiency from the press of a finger, thus achieving synergistic energy collection (FIG. 23D). As a practical application, such efficient hybrid harvesters are used to power an electronic sensing system that contains vitamin C or sodium ion sensors with dedicated low-power electrochromic display (ECD) to operate independent from external devices (FIG. 23E). Overall, the described touch-basedBFC harvester demonstrates extremely high harvesting efficiency and EROI compared to any previously reported on-body bioenergy harvesters (Table 1 below). The paradigm shift from “work for power” to “live to power” enhances the practicality of existing on-body bioenergy harvesting technologies, and offers new and unique possibilities of establishing reliable and independent next-generation self-sustainable electronics systems.

Characterization and Optimization

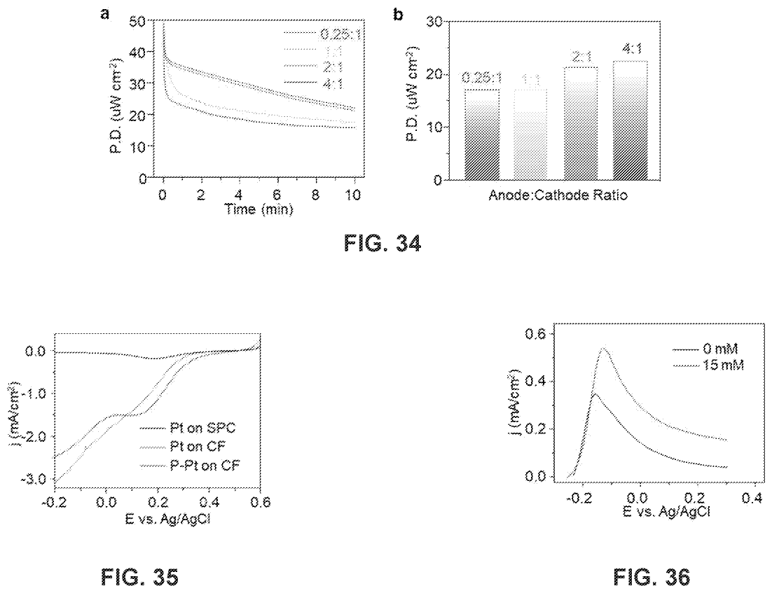

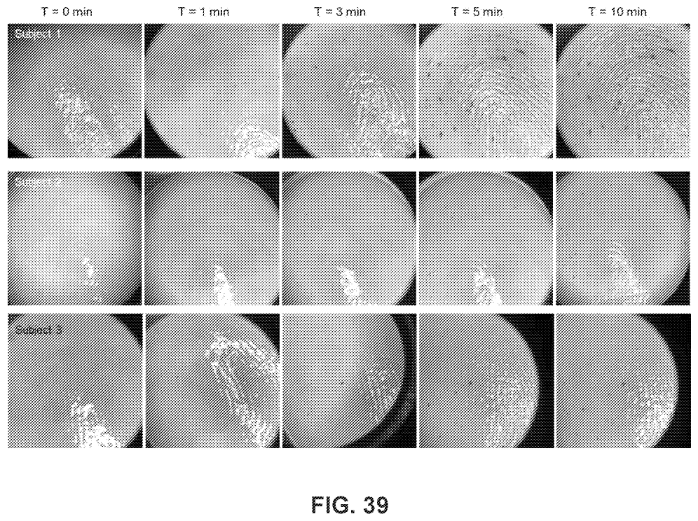

The fabrication of a touch-based BFC that effectively utilizes the natural fingertip sweat pumping, under repeated pressing, relies on soft, durable, porous, sweat-wicking CNT foam electrodes. These flexible CNT foam electrodes are prepared by using a water-soluble particle template and solvent exchange in a formulated CNT-elastomer composite. Through optimization (FIG. 34), the CNT-foam-based fingertip BFC is designed with the total size of 1×1 cm², with one cathode electrode paired with two anodes (FIG. 23B). The operational conditions of the BFC are optimized first using in-vitro tests. The BFCs are traditionally characterized using linear scan voltammetry (LSV) with the scan rates around 5 mV s⁻¹, which is used to gauge the power of the BFC against different fuel concentrations and the peak power potential (FIG. 24 (a)). However, such method cannot accurately depict the long-term harvesting performance in equilibrium since capacitive charging current makes up a significant part of the measured power output. Thus, extended chronoamperometry (CA) of 10 min is employed, with potential steps from 0.55 V to 0.2V to accurately evaluate the power and optimal operating conditions of the BFC. The open-circuit voltage of the cell demonstrated a value of ca. 0.55 V, in agreement with the onset potential of LOx/NQ-driven oxidative and Pt-catalyzed reductive reactions, set at −0.2 V and 0.35 V vs. Ag/AgCl, respectively (FIGS. 35-36). The Pt-based ORR is chosen as a cathodic half-cell over enzymatic biocathodes to minimize the risks involved in enzyme immobilization and eliminate energy harvesting fluctuations due to environmental changes. A potential of 0.40 V offered the most favourable performance with the power density of 43 μW cm⁻² (based on the anode area) in the presence of 15 mM lactate concentration (FIG. 24 (b)). Expectedly, the response of the BFC to increasing lactate concentrations, ranging from 1 mM to 25 mM, in both the liquid PBS and the PVA hydrogel media (under 50 kPa applied pressure), has been tested at the optimized (0.4 V) potential (FIG. 24 (c) i and ii) and led to higher bioelectrocatalytic currents. FIG. 24 (c) ii demonstrates that compared to the PBS medium, the additionally applied pressing pressure of 50 kPa using the PVA gel results in a slightly higher power output. This behaviour, characterized with electrochemical impedance spectroscopy (EIS), is attributed to the low impedance of the porous PVA gel as well as to the reduced electrode resistance of the electrodes upon applying pressure (FIG. 38).

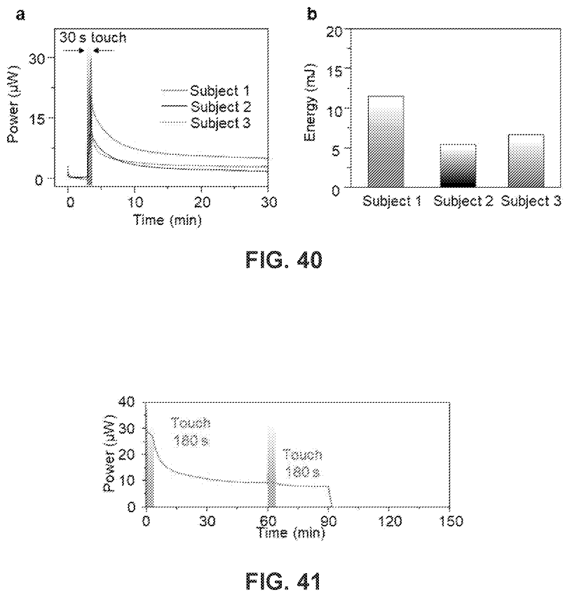

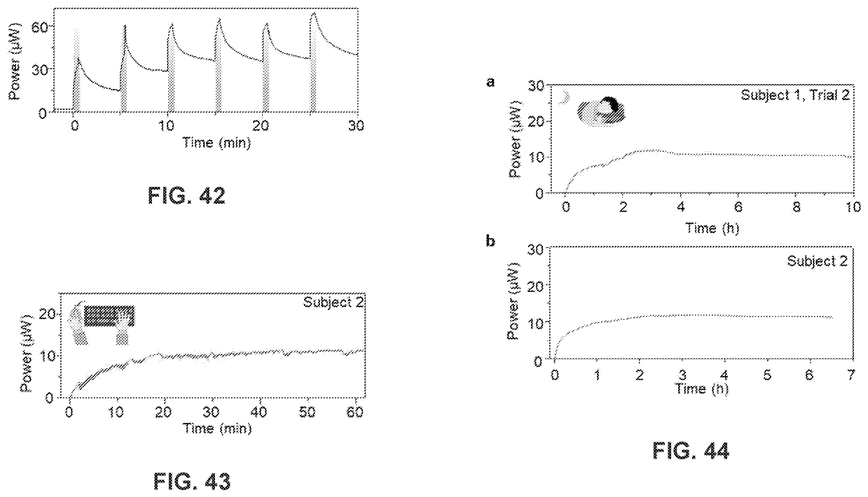

FIG. 24 (d) shows an example proof-of-concept power response of the touch-based BFC. This power-time temporal profile displays a rapidly increasing power, to around 30 μW, upon pressing the BFC with a bare finger (green section). In comparison, no power generation is observed for similar touching of the BFC using a covered finger (black section), reflecting the absence of fuel transfer. Such comparison clearly demonstrates that the power generation in a BFC is sheerly fuelled by the fingertip's natural sweat. Due to the difference in sweat rate and lactate concentration in different individuals, the harvestable power can vary from person to person, giving an advantage of BFC-produced power to individuals with higher fingertip sweat rate (FIGS. 39-40). The harvesting behavior of BFC during continuous pressing is further validated over a 30 min period, which generated over 20 μW of power per finger and harvested over 39.5 mJ energy over 30 min (FIG. 24 (e)). In comparison, the BFC without LOx enzyme is not able to generate any sizable energy. The ability of the touch-based BFC to continuously harvest energy from the sweat transferred from a brief (3 min) touch is demonstrated in FIG. 24 (f), where the BFC is able to harvest energy over an hour and can be refuelled upon touching the porous PVA hydrogel. It is worth noting that without enclosure, the collected sweat fails to maintain the PVA gel hydrated due to faster evaporation kinetics and the gel is rehydrated every hour (FIG. 41). As shown in FIG. 24 (g), repeated and frequent pressing on the BFC is beneficial for increasing its harvesting power, with its power increasing after each press. Such behaviour can be utilized to increase rapidly the power harvested without exerting constant force upon the device and can be further exploited with more BFCs for multiple fingers to reach higher power (FIG. 42). The simplicity and practicality of this touch-based BFC harvester are demonstrated in different scenarios, such as during regular desk work that involves typing and mouse-clicking (FIG. 24 (h) and FIG. 43), or during overnight sleep when no mechanical input is exerted (FIG. 24 (i) and FIG. 43). These data demonstrate that the BFC is able to harvest energy continuously in both scenarios, scavenging over 28.4 mJ during 1 h of desk work, or up to 389 mJ energy over 10 h of sleep, without the need for any environmental or mechanical energy input.

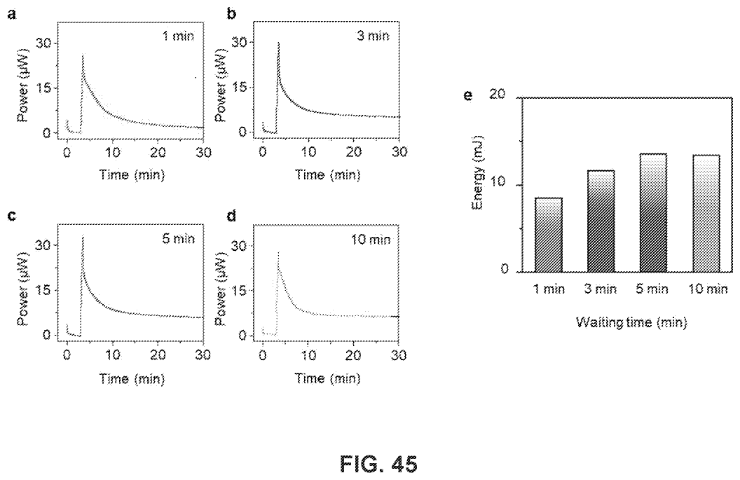

To further investigate and optimize the utilization of the touch-based bioenergy harvesting process, several variables that affect the power generation, including the sweat accumulation time (after cleaning and prior to the touching), the touching pressure, the duration of touching, the number of fingers employed, and the touching frequency, have been systematically studied. Towards the practical goal of quickly powering a device within a short period after touching, the power and the total energy generated during a 5-minute touching are monitored and compared. First, the effect of the sweat accumulation time before touching the BFC is examined using a 1 to 10 min time range and the corresponding power generation is monitored during a 30 sec touching time. While longer waiting times are expected to increase the power due to accumulation of lactate on the fingertip, no significant difference in the power is observed for the different waiting times (FIG. 25 (a)). Such difference, however, is more pronounced through a longer operation time of 30 min, when the stabilized power and total energy collected from BFC increased slightly upon increasing the waiting time (FIG. 45). Subsequently, the effect of the applied finger pressure on the BFC performance can be shown by touching the 1 cm² device with increasing pressures of 10, 25, 50, and 100 kPa. FIG. 25 (b) shows that a stronger press force leads to a higher power, which translates to a larger harvested energy within a fixed time. In this example implementation, the pressure weight of 50 kPa was determined as the most appropriate since applying a larger pressure required extra effort with only negligible gain in the energy payback.

Example embodiments of the sweat permeation layer including the hydrogel, such as the example porous PVA gel in FIG. 25(b), has a structure that facilitates the transfer of naturally-produced sweat (containing the analyte) from the fingertip of the subject, such that the device does not require sweat inducement, whether by requiring the subject to exercise or otherwise generate heat to induce sweating or by requiring an iontophoretic effect or a chemical stimulate to induce sweat production from the user. The sweat permeation layer including the hydrogel is capable of permeating the naturally-produced sweat (including in low volume, e.g., microscopic droplets) across the side of the sweat permeation layer in contact with the fingertip to the side in contact with the sensor (e.g., electrodes). Furthermore, the sweat permeation layer is structured to enhance the quality of the detectable electrical signal from the facilitated electrochemical reaction of the analyte in the permeated sweat (e.g., tiny droplets having a dimension in the tens or hundreds of nanometers or tens or hundreds of microns). For instance, the applied pressure by the user's finger minimizes diffusion pathways and reduces electrode impedance (see example data of FIG. 38) at the detecting electrode, e.g., based on an increased electrical conductivity of the example carbon-foam-based BFC structures and porous hydrogel layer (e.g., PVA gel) when under compression. Moreover, due to the flexible and durable structure of the porous carbon-foam-based BFC and a porous PVA gel, for example, no mechanical damage is observed throughout the process of compression of the sweat permeation layer.

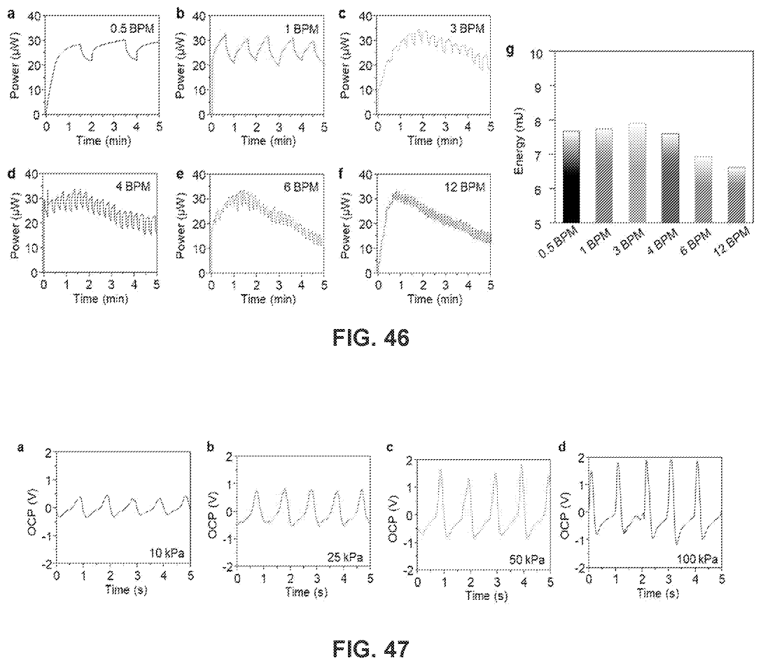

As shown in FIG. 25 (b), the power harvested from fingers is directly proportional to the number of fingers with device deployed with different pressing duration (30 s and 3 min), where the 3-min pressing with 3 fingers harvested as high as 17 mJ over 5 min, translating to an average power of 56.7 μW and energy ROI over 1000% considering the small amount of energy (˜0.5 mJ/finger/press) used in pressing the fingers. As is shown earlier in FIG. 24, pressing can increase the instantaneous power of the BFC, with the press time affecting the total amount of energy harvested within a short time period. As expected, the BFC pressing time profoundly affects the energy generation (FIG. 25 (d)). These data display the power-time profiles recorded upon increasing the BFC touching time from 5 to 180 s. Such profiles show that longer touching times lead to higher power generation and the collection of additional energy, indicating that the sweat is able to continuously diffuse through the gel during the touching period. To further examine the benefit of repeated fuelling to the power harvesting within a short amount of time (towards quickly powering electronics), the 180 s touching time and the remaining 120 s idling time are divided into 5, 15, 30, and 50 segments, which correspond to touching frequencies of 1, 3, 5, and 12 beats per minutes (BPM), respectively. Such characterization is crucial also for further integration with PENG harvesters that requires repeated pressing for energy harvesting. As shown in FIG. 25 (d) and FIG. 46, the total energy harvested within 5 min increases to a total of 8 mJ per finger at a touching frequency of 3 BPM and starts to decrease upon raising the BPM to 12, reflecting the rapidly decreasing period when the finger is taken off from the gel.

Integrated Touch-Based Energy Harvesters

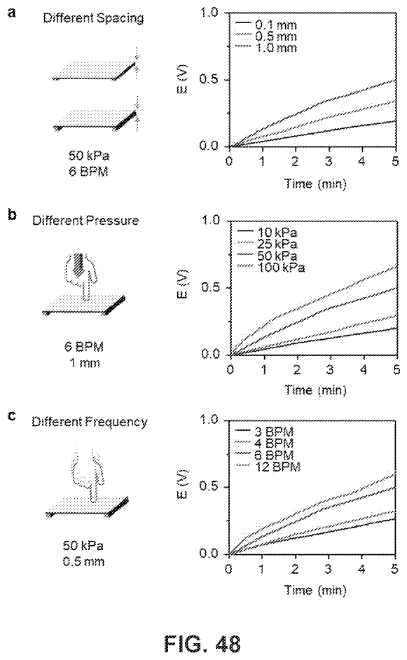

After optimizing the operation of the fingertip BFC, the potential of the efficient bioenergy harvesting approach towards practical autonomous and sustainable powering of wearable devices is evaluated. To ensure the applicability of the self-powered device, the system is expected to store a sufficient amount of the harvested energy with the ability to boot the electronics as quickly as possible for the pulsed operation mode. To this end, the energy input from the harvesters, the energy storage for regulation, as well as the system energy consumption have to be characterized carefully along with budgeting of the energy flow for ensuring highly efficient system operation. The energy harvesting capability of the BFC is thus tested first via charging a capacitor that can be subsequently used for powering electronics in a pulsed manner. Due to the low potential input from the BFC, a low-power booster with energy regulation circuit is designed to boost the BFC voltage for charging the capacitor up to 4 V. Furthermore, to fully exploit the energy input associated with the finger pressing action, a PZT-based PENG has been integrated with the BFC in a judicious layout using the same device footprint—to harvest the corresponding mechanical energy simultaneously. Such integration allows the synergistic harvesting of bioenergy associated with the same finger-pressing motion and requires careful considerations of the characteristics of the individual harvesters to maximize their power generation while minimizing their limitations. Due to the PENG's high alternating voltage nature, its input is regulated via a bridge rectifier before connecting to the capacitor. The system diagram of the integrated BFC-PENG harvester is shown in FIG. 26 (a). The PENG's energy harvesting relies on the mechanical deformation of the PZT chip, located directly below the BFC energy harvester, and is activated upon a slight finger press. The power generated on the PZT increases upon raising the pressing force, frequency, and deformation (controlled by the thickness of the spacer) (FIGS. 47-48). Therefore, and as shown in FIG. 26 (b), the best performance of the integrated system is expected at a touching frequency exceeding that of the BFC system alone. After the successful integration of a single set of the mechanical and biochemical energy harvesters (FIG. 26 (c)(i)), an identical set of PENG harvester is attached to the opposite side of the BFC, in a sandwich-like manner, to effectively harvest the mechanical energy through a pinching motion, hence harvesting the maximum amount of power without expanding the device footprint (FIG. 26 (c)(ii)). Adopting the optimal pressing frequency of 6 BPM and a pressure of 50 kPa, the charging rate of BFC is tested against external capacitors ranging from 47 to 470 μF (FIG. 26 (d)). The capacitors' charging time increases upon increasing the capacitance, with the prevailing contribution of BFC as the primary energy source. To gauge the charging behaviour of the energy harvesters, a 100 μF capacitor with a set voltage window between 2 V and 4 V is used to examine their corresponding charging times. Whereas the independently acting biochemical and mechanical energy harvesters are able to charge the selected 100 μF capacitor within 8 and 20 min, respectively, the integrated system completed this task within only 4 minutes (FIG. 26 (e)). It should be noted that such synergistic behaviour is expected to surpass the mere addition of the power from both harvesters, as the increase in the total energy input enhances also the conversion efficiency of the booster circuit.

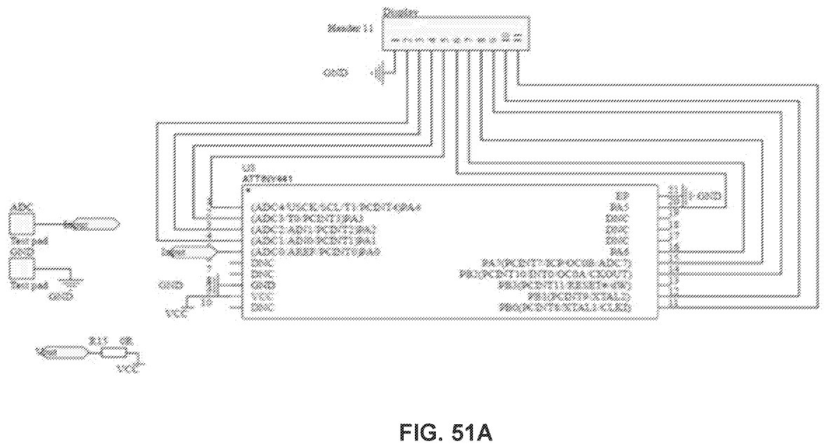

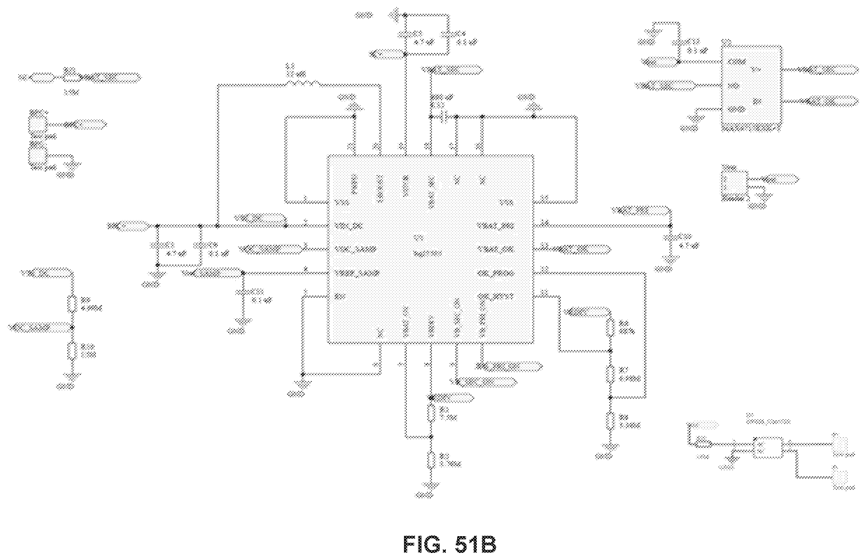

Referring to FIG. 26 and FIG. 27A-27B, an energy management circuit includes a low-power booster (e.g., booster 2708) that can be configured as a DC-to-DC boost converter, which steps up voltage and steps down the current to supply sufficient voltage (e.g., >2V) to power electronic devices. The example BFC 2704 in FIG. 27B has an input voltage up to only 0.5-0.7 V, and for its powering of electronics, the boost converter (e.g., booster 2708) was designed to lift its output voltage and store such voltage in an energy storage device (e.g., capacitor, supercapacitor, battery, etc.). An integrated circuit containing the boost converter as well as a charging regulator, which prevents over-charge and over-discharge of the energy storage device (like that shown in FIG. 51B), can be used to regulate the power output from the biofuel cell for its subsequent powering of electronics such as a microcontroller unit (as shown in FIG. 51A).

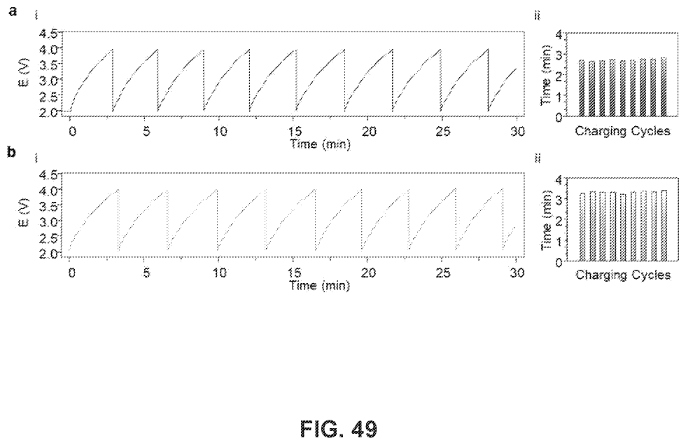

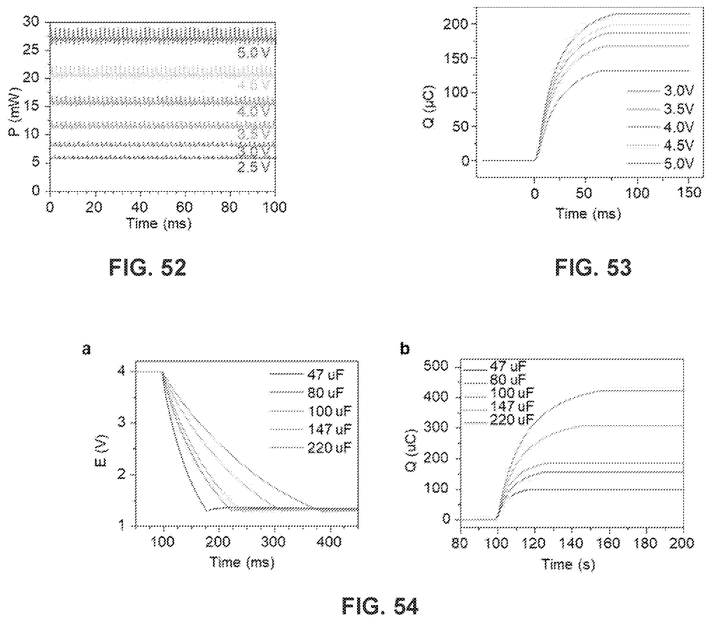

The energy harvesting operation is also optimized in terms of the pressing frequency of the finger. As is discussed earlier, the 50 kPa pressure is found to be optimal in terms of convenience-to-power output ratio. Therefore, the influence of the touching frequency upon the bioenergy harvesting is evaluated using the 50 kPa pressure at pressing frequencies of ranging from 1 to 24 BPM to determine the optimal pressing frequency that can charge the 100 μF capacitor in the shortest time. As shown in FIG. 26 (f), a charging rate of 6 BPM pressing pattern offers faster charging of the capacitor compared to the 3 and 12 BPM pressing frequencies, and leads to the fastest charging speed. The trend observed in FIG. 26 (f) is in agreement with the profiles shown in FIG. 26 (b), as the lower frequency (3 BPM) provided less mechanical energy input for the PZT, while the higher charging frequency reduces the biochemical energy harvesting efficiency (FIG. 25 (e)), leading to an optimal charging rate at 6 BPM with the lowest charging time. With the optimized pressing frequency of 6 BPM, the performance of a single BFC harvester can be compared to its sandwiched configuration (two back-to-back integrated devices), FIG. 26 (g). As expected, the double-sided harvesting device, employing two fingers' pressing motions for energy harvesting at 6 BPM, charges the 100 μF capacitor to 4 V within about 2 minutes, compared to the 4 minutes charging time observed with the single PZT-integrated BFC setup. Lastly, the continuous energy-harvesting ability of the double-sided BFC-PZT is examined at the previously optimized conditions (50 kPa at 6 BPM). As shown in FIG. 26 (h)(i)-(ii), two sets of BFC harvesters can effectively and consistently charge the capacitor in ˜ 2.8 min over the 30 min period. Similarly, the integrated BFC-PZT harvester pair also delivered consistent energy production, offering a faster charging time of about 2.3 min (FIG. 25 (h)(iii)-(iv)). The charging process is also tested on a subject with lower sweat rate, which results in a slight increase in the charging time (FIG. 49). Furthermore, in order to further reduce the charging time of the system, a similar amount of charge can be harvested at a lower voltage employing capacitors with larger capacitance. As shown in FIG. 26 (h)(v)-(vi), a 220 μF capacitor is charged in the voltage window between 2 V and 3 V, which takes only about 92 s, and is significantly faster compared to charging 100 μF capacitor to 4 V. Such change can be beneficial to the rapid powering of electronic devices, and the lower voltage can also limit the power consumption of MCUs (FIG. 52). The PZT-integrated sandwiched BFC system is shown to be the most efficient, continuously and repeatedly charging the capacitor following by its polarization. The integrated system allows substantial energy harvesting using the pinching motion and natural sweat flow with negligible energy input from the fingertip. Considering the energy input of pressing the fingers every 10 s (˜1 mW), such energy harvesting behavior is attractive compared to the typical tribo/piezoelectric harvesters and BFCs that require movements or exercise as energy input (>100 W). These results exemplify the potential of the hybrid BFC-PZT harvesters integration for practical applications, demonstrating the most favorable energy ROI ever reported among all bioenergy harvesters and setting new standards in the bioenergy collection efficiency of wearable harvesters.

Self-Powered Sensing System

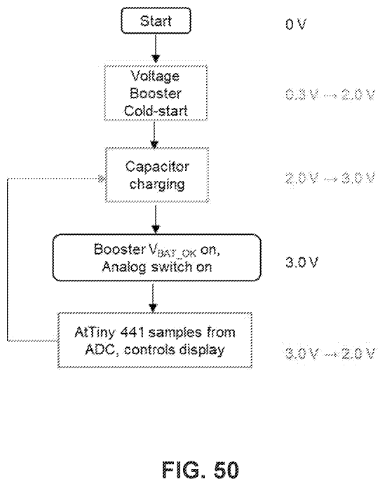

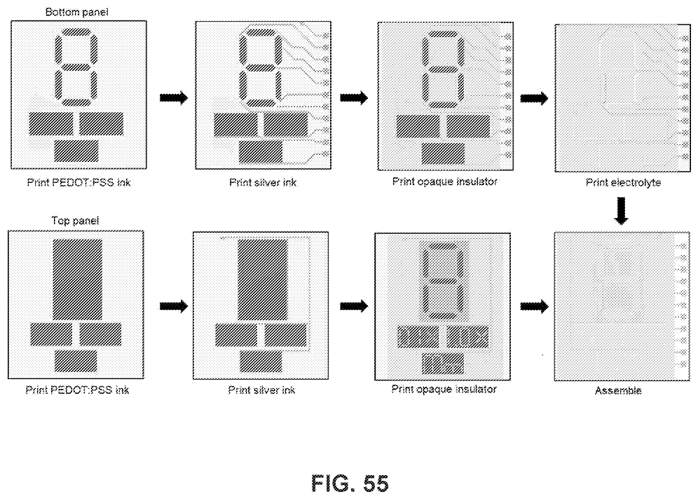

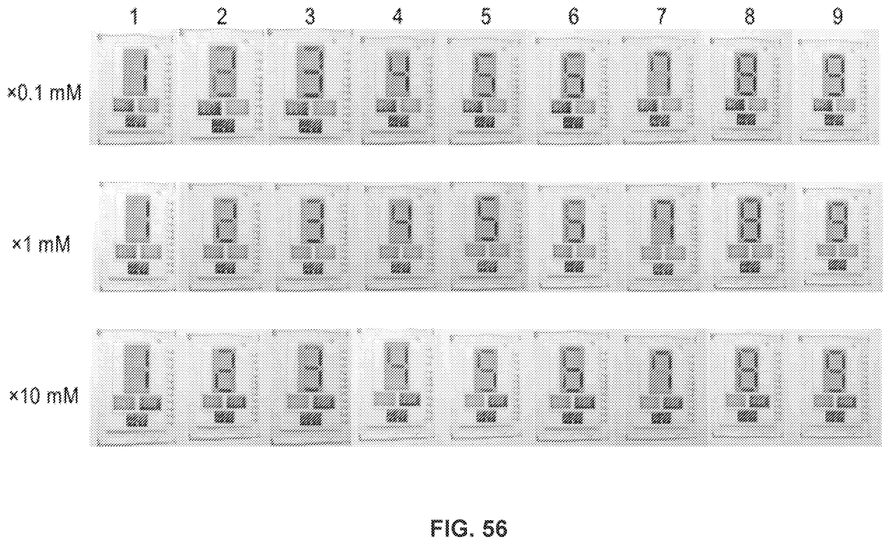

Referring to FIG. 27B, the self-powered sensing system implemented based on some embodiments of the disclosed technology includes an energy harvester 2701 and an energy management circuit 2705, a microcontroller unit (MCU) 2714, an electrochromic display (ECD) 2716, and a sensor 2718. In some implementations, the energy harvester 2701 includes a piezoelectric generator 2702, such as PZT, and a biofuel cell (BFC) 2704, such as a touch-based biofuel cell. In some implementations, the energy management circuit 2705 includes a bridge rectifier 2706, a voltage booster circuit 2708, an energy storage device 2710, and an analog switch 2712. To demonstrate the practical utility of the finger-based integrated bioenergy harvester for powering electronics, a potentiometric sensing system with an ECD panel, operated in pulsed sessions, is developed (FIG. 27A). Such a system is composed of the energy regulation components that separately manage the low-voltage, continuous input from BFCs 2704 via a booster circuit 2702, and the high-voltage, alternating, and pulsed input from PZT 2702 chips via a bridge rectifier 2706 (FIG. 27B, FIGS. 50-51). Both rectified energy inputs are collected in the energy storage device 2710 such as a capacitor, a supercapacitor, or a battery. The overvoltage protection function of the booster circuit 2702 is utilized, which is connected to an analog switch 2712 that controls the supply energy to an MCU 2714 from the capacitor 2710. In some implementations, the energy regulation components may further include a regulation circuit to regulate output voltage of the booster circuit 2702 (e.g., to prevent over-charge and/or over-discharge). A low-power MCU 2714 is chosen with a 10-bit analog-to-digital (ADC) resolution to read the voltage input from the sensor and control the “on” and “off” of 10 individual ECD pixels. The ECD, fabricated via layer-by-layer screen-printing (FIG. 27C (i), FIG. 55), is chosen for its low energy consumption, as it requires energy only while refreshing the displaying content. The pixels contain a 7-segment number display, along with two pixels for displaying the range (“×0.1” and “×10”) of the sensing and one pixel displaying “mMf” as the unit of the chemical sensing when the system boots for the first time (FIG. 27C (ii), FIG. 56). The system design obviates the integration of any wireless communication electronics, as such system would require external electronics (e.g., smartphone, smartwatch, computers) for data transmission and processing for obtaining the sensing results. To maximize the system energy utilization efficiency while ensuring the operation of the ECD, the power and charge consumption for the MCU and the ECD are carefully characterized, the capacitance for energy storage is optimized at 220 μF, and the operation window of 3 V-2 V (FIGS. 52-54 and 57). Two sets of integrated BFC-PZT harvesters configurated back-to-back are connected to the system to supply the harvested biochemical and mechanical energies from the pinching motions of the thumb and the index finger. Sensors can be connected to the system ADC channel for data acquisition, and the results are displayed via the ECD in the resolution of 1 significant figure (Tables 2-3 below).

Two types of sensors are employed for demonstrating the applicability of such a self-powered sensing system: a potentiometric sodium sensor and a vitamin-C sensor. The potentiometric sodium sensor relies on measuring the potential difference between the sodium-ion-selective membrane on the working electrode and the silver/silver chloride (Ag/AgCl) reference electrode when in contact with the sodium sample solution (FIG. 27D (i)). The electrode-electrolyte interface results in a sodium concentration gradient (between the membrane and the solution) leading to a potential signal that depends logarithmically on the sodium concentration. Such potentiometric sensing applies to a wide range of clinically or environmentally important electrolytes. FIG. 27D depicts the calibration of the fabricated sodium sensor, demonstrating a slope of 59.3 mV per decade of sodium concentration. It also indicates a good selectivity against potassium ions, which display a negligible change in the sensor potential. As shown in FIG. 27E, the system can boot upon pressing the energy harvesters monitoring different sodium concentrations in tap water and 1:100 diluted sea water.

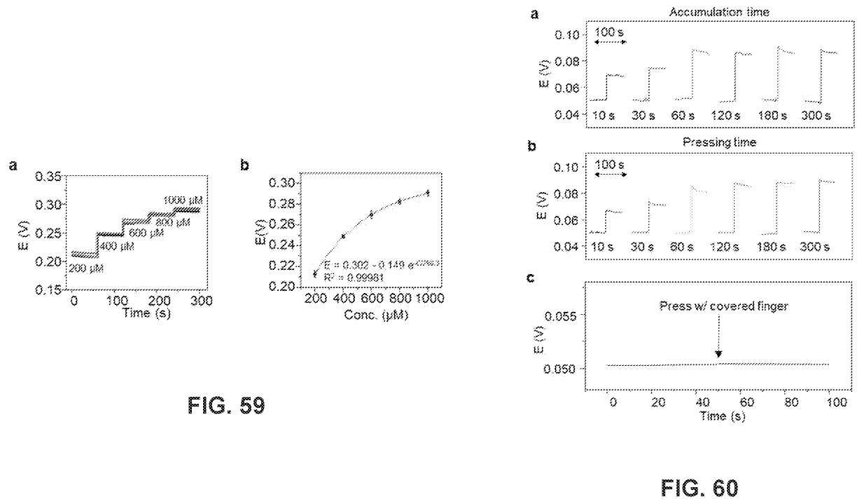

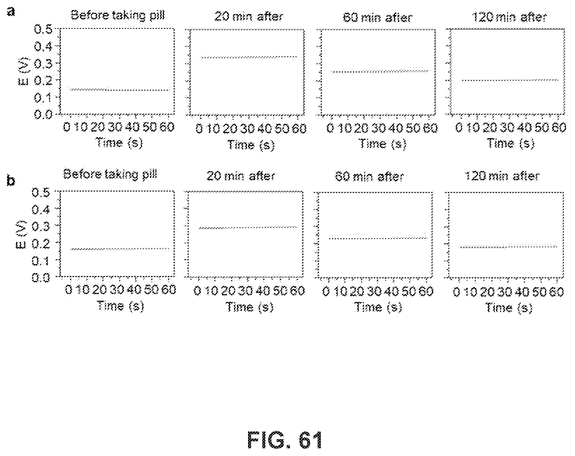

Vitamin C sensing commonly relies on amperometric measurements converted here into potentiometric ones via a controlled load. Such sensors, usually referred to as “self-powered” sensors, rely on the autonomous oxidation reaction on the working electrode along with a complementary reduction reaction on the counter electrode, analogous to those of BFCs (e.g., enzymatic glucose, lactate, or alcohol sensors). In this case, the sensing principle is based on electrocatalytic oxidation of the vitamin, generating a proportional current flow, which is further converted into a potential difference signal (ΔE) under the applied load. The vitamin C sensor relies on the selective, non-enzymatic oxidation of ascorbic acid (AA) on the anode catalyzed by the immobilized tetrathiafulvalene-tetracyanoquinodimethane (TTF-TCNQ) charge-transfer complex; silver oxide (Ag₂O) is used as the cathode material, which delivers a stable potential throughout its reduction (FIG. 27F (i)). The sensing of vitamin C in stimulated sweat is described previously and is adapted here to detect the vitamin C levels in the natural fingertip sweat. The load between the two electrodes is optimized at 10 MΩ. The calibration experiment in FIG. 27F (ii) and FIG. 59 demonstrates the sensitivity of the vitamin-C sensor, while the corresponding interference study shows the high selectivity of the sensor against common sweat constituents, including glucose, urea, lactate, and acetaminophen. For this, a hydrogel, similar to that used in the BFCs, is pre-soaked in artificial sweat and placed over the sensor to absorb the fingertip sweat upon touching. The on-body usage of the touch-based vitamin C sensing is optimized for sweat generation time (60 s) and sweat collection time (120 s) (FIG. 60). As shown in FIG. 27G, a human subject is asked to take a vitamin pill and sense the vitamin C level continuously over 30 min. The ECD can quickly update the resulting vitamin C concentration (every 1-2 min), and the sensing system is able to capture the dynamics of the rise and fall of the vitamin C concentration within the natural fingertip sweat (FIG. 61). Unlike other studies that use various energy harvester for self-powered sensing and require rapid movements and rigorous exercises, the present system can boot rapidly and continuously, and efficiently harvest energy from the slow pressing action of fingers and effortlessly supply power to complex electronic systems. Thus, the integrated harvesting system has shown its distinct advantage in practical application as an independent, self-powered electronics system toward personalized health and nutrition wellness, or environmental monitoring.

The disclosed technology can be implemented in some embodiments to provide a biofuel energy harvester with extremely high energy ROI, that effectively harvests energy from the natural fingertip sweating and the fingers' pressing motion, and its practical application in self-powered and fully integrated sensing device. The demonstrated concept of utilizing continuous naturally pumped sweat and intuitive finger pinching motion for energy generation and operation of low-power electronics shifts the current paradigm of bioenergy harvesting devices from “work for power” into “live to power”. This concept is demonstrated by energy harvesting while sleeping or low-intensity desk work, converting traces of kinetic and chemical energies, resulting from our daily activity, into electric form. Utilizing the effortless and continuous fingertip sweating as the energy source, the BFC harvester is further boosted by a piezoelectric PZT harvester that fully exploit the intuitive finger motion of pinching. With a small footprint of 2 cm², this system delivers similar energy collection performance while exhibiting a high energy harvesting efficiency compared to any previously reported bioenergy harvesters which require vigorous motions or extreme sweat-inducing exercises. Pairing a low-power ECD with the touch-based harvester platform presented an energy-efficient electrochemical sensing system that can be applied to a wide variety of sensors for personalized health and nutrition monitoring applications, beyond the demonstrated sweat vitamin C and sodium sensors.

The integrated system has been designed around smart and highly efficient utilization of limited bioenergy to realize a fast-responding, extended, and autonomous operation in connection to complementary, synergistic harvesters, optimized energy storage units, low-power energy management integrated circuit, MCU, and displays. The possibility of utilizing the passive sweat for self-powered sensor can added, where the sensor's power or open-circuit voltage can be correlated with the concentration of the target analyte in the sweat. Such highly efficient, user-friendly biocompatible energy harvesting technology, coupled with the system integration and corresponding judicious energy budgeting, offers considerable promise for establishing self-sustainable, reliable, and independent next-Generation Epidermal Electronics Systems for Tracking Healthcare and Wellness.

Example Fabrication Techniques

Fabrication of the Example Flexible CNT Foam

The fabrication procedure of the flexible CNT foam is described in FIG. 28. In detail, MWCNT-COOH (0.25 g), graphite (0.05 g), and NaHCO₃ (6.0 g) are mixed well with motor and pestle to obtain gray-colored powder followed by adding 3.0 g of SEBS (dissolved in toluene with 4 (SEBS):10 (toluene) wt %) and stirred at 1800 rpm for 5 min. Subsequently, 2.5 ml of toluene is added and mixed again at 1800 rpm for 5 min to attain homogeneous paste. The resulting paste is then cast in 1 mm high rectangular-shaped structure, carefully controlled by placing two glass slides (1 mm thick) with controlled spacing in between. Immediately, the cast portion is transferred to ethanol for 20 min to make a solvent exchange that prevents collapse of the structure during evaporation of the solvent (toluene) and then dried in ambient conditions. After that, the dried CNT foam is soaked in 0.5 M HCl for 3 hours to completely remove the NaHCO₃ template; this process resulted in the highly porous structure of the CNT foam. The resulting porous CNT foam is washed with distilled water for several times and dried at 80° C. to obtain flexible CNT foam.

Fabrication of the Example Biofuel Cell

Each CNT foam is cut into 0.3 cm² (1 cm×0.3 cm) and two of them (for anodes) are immersed in 10 mM EDC/NHS solution for 6 h to activate the carboxylic acid groups of the MWCNT. After washing the CNT foams with DI water for several times, they are attached on the silver current collector with carbon ink placing the cathode between the two anodes. Each bioanode is fabricated by drop casting 10 μl 0.2 MNQ (dissolved in 1:9 ratio of acetone: ethanol), followed by the addition of LOx (40 mg ml⁻¹ in 10 mg ml⁻¹ of BSA, 10 μL) for 3 h. For immobilizing the enzyme, 5 μl each of 1% chitosan in 0.1 M acetic acid and of 1% glutaraldehyde are drop cast on the anode then kept in 4° C. overnight. Otherwise, the cathode is fabricated by a fixed-potential co-electrodeposition of Pt and Cu at −0.75 V for 600 s followed by de-alloying the Cu with cyclic voltammetry over the potential range of 0 V to 1.5 V for 40 cycles (scan rate 50 mV s⁻¹). After rinsing with DI water for several times, 1% of Nafion is drop cast on the cathode and kept in the room temperature until use.

Fabrication of the Example Porous PVA Hydrogel

The fabrication of the porous PVA hydrogel is adapted from previous studies. Firstly, solutions of the PVA dissolved in water in a 1:10 weight ratio and KOH dissolved in water in a 1:5 weight ratio are prepared. Then, 14 g of the KOH solution is added dropwise to 10 g of PVA solution with stirring, followed by dissolving 2.6 g of sucrose into the mixture to form the hydrogel precursor. 15 g of the precursor is then poured into a Petri dish (diameter ˜9 cm) and left in a vacuum desiccator to remove excess water and allow crosslinking until only ⅓ of the weight of the precursor is left. The crosslinked gel is then soaked in 0.1 M PBS buffer to remove the sucrose template and the excess KOH, until the gel is in neutral pH. The gel can then be cut into desired sizes and shapes and stored in PBS or AS for subsequent use.

Fabrication of the Example Electrochromic Display

The ECD is designed using AutoCAD and screen-printed layer-by-layer onto SEBS sheets. The design of the ECD is separated into a front panel and aback panel, which are separated by a layer of white, opaque insulator and PSS electrolyte, and assembled via heat sealing.

Fabrication of Example Sensors

The sodium sensor is fabricated using flexible silver and carbon inks. The silver ink and the carbon ink are printed onto SEBS substrate layer-by-layer, and are covered using SEBS resin to defined the electrode area, exposing 2 mm² of carbon electrode as the working electrode and 1 mm² of silver electrode as the reference electrode. A 0.1 M FeCl₃ solution is firstly drop-cast onto the silver electrode to chlorinate the surface and form AgCl.

A cocktail composed of PVB (78.1 mg ml-1) and excess amount of potassium chloride (50 mg ml⁻¹) dissolved in methanol is drop-cast onto the chlorinated surface (1.5 μl mm⁻²). A PU resin (1 g in 10 g TIF) is then drop-cast onto the dried cocktail layer (2 μl mm⁻²) to prevent salt leaching. A cocktail for the sodiumion-selective electrode is formulated by dissolving 1 mg of sodium ionophore X, 0.77 mgNa-TFPB ion exchanger, 33 mg PVC, and 66 mg DOS in 660 mL nitrogen-purged THF, and drop-cast onto the carbon electrode (2 μl mm⁻²).

The vitamin C sensor is fabricated using flexible silver, carbon, and silver oxide inks. The inks are printed layer-by-layer onto a SEBS substrate and covered using SEBS resin to define the electrode area, exposing 2 mm² of carbon electrode and 4 mm² silver oxide electrode. A 10 MΩ resistor is solvent-welded between the two electrodes as the discharging load. A 5 mM solution of TTF-TCNQ, dissolved in ethanol:acetone (1:1) mixture, is drop-casted onto the carbon electrode (1 μl mm⁻²), followed by drop-casting a 1 μl mm⁻² chitosan layer (1 wt % in 0.1M acetic acid) and a 0.125 μL mm⁻² glutaraldehyde layer (0.5% in water) for immobilization.

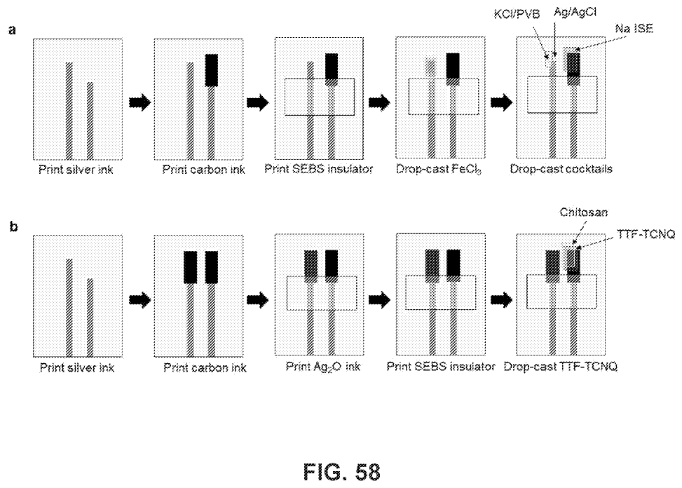

More detailed fabrication process of the sensors is shown in FIGS. 58-61.

Example Electrical Circuit Design

The circuit is composed of four main components: MCU, analog switch, booster, and bridge rectifier. The PCB design is shown in FIG. 51. Individual components are then soldered onto the PCB via standard reflow process. The integrated circuit could perform energy harvesting, storage, and the power management. The MCU with a built-in ADC could read from the sensor and display the corresponding result via the ECD.

Assembly of the Example Self-Powered Sensing System

An adaptor that connects two sets of BFCs and PZT chips are designed using AutoCAD and screen-printed onto a SEBS sheet (FIG. 31). The front and the back PZT chips are separately connected to the adaptor and the two PZT chips are placed back-to-back, separated by two spacers (1 mm thick) place on two ends of the chips. The foam BFC electrodes are thereafter fixed onto their corresponding locations using a conductive carbon ink and modified using the procedure above. The connector is then connected to the PCB via the “solvent welding process” following previous studies. Similarly, the display and the sensor is connected to the PCB using the same process to complete the assembly of the self-powered sensing system.

Highly Efficient Fingertip Biofuel Harvesting System: Towards Autonomous Self-Powered Sensing and Display

FIG. 28 shows synthesis of the CNT foam: (a) CNT composite paste preparation; (b) the fabrication steps of the CNT foam using the paste.

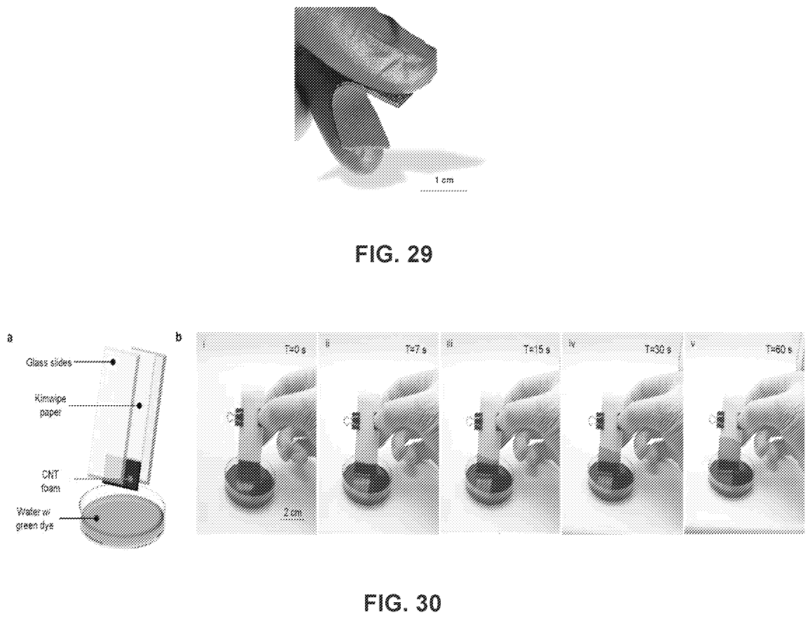

FIG. 29 shows photographic image of bending a strip of 1×3 cm² CNT foam.

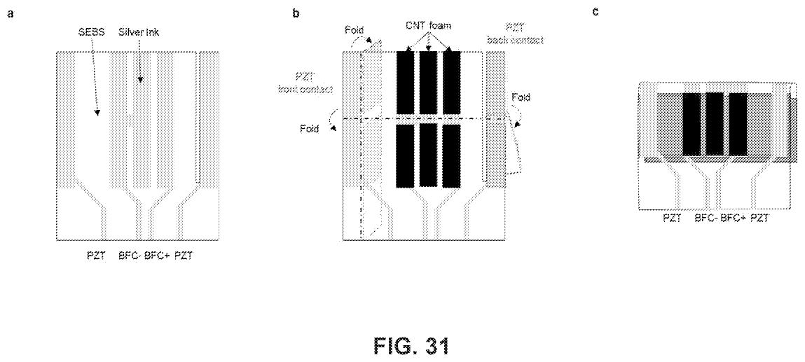

FIG. 30 shows water wicking performance of the CNT foam: (a) schematics of the water-wicking test of the carbon foam. A piece of 1.5 cm×2.5 cm CNT foam is sandwiched between two glass slides with Kimwipe paper (same thickness as carbon foam) on top of the foam. A plate with water is prepared with green dye for visibility; (b) the time-lapse photographic images at 7 s (ii), 15s (iii), 30 s (iv), and 60 s (v) after dipping the CNT foam into the water. The water successfully penetrated through the CNT foam within 7 s.

FIG. 31 shows an assembly of the CNT foam for BFC and PZT chips: (a) current collectors are firstly printed onto a SEBS sheet, and trimmed to the shape; (b) the CNT foam pieces (1 cm×0.3 cm) are attached to the silver current collector using carbon composite ink; (c) attaching two PZT chips to their corresponding contact points, and folded back-to-back.

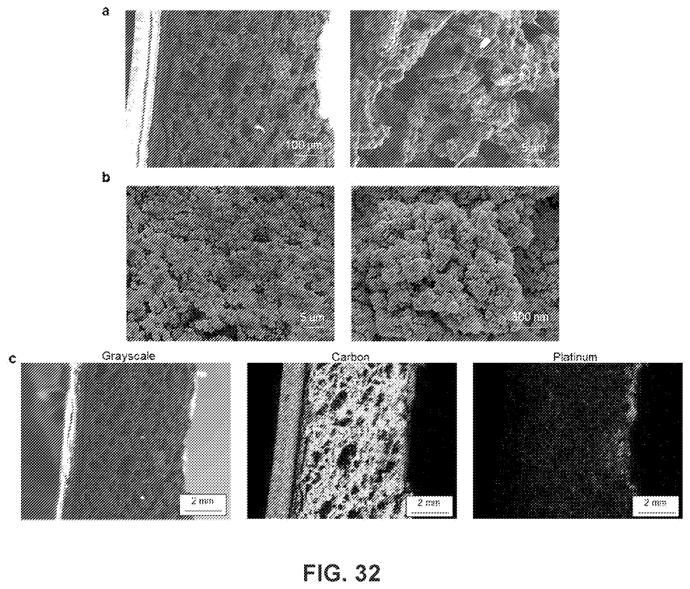

FIG. 32 shows SEM images and corresponding EDS mapping of the CNT foam cathode: (a) cross-sectional view of the p-Pt CF cathode; (b) front view of the p-Pt-CF cathode; (c) EDS mapping of carbon and Pt on the cross-section of the p-Pt CF cathode.

FIG. 33 shows cryo-SEM images of the cross-sections of the porous and non-porous PVA hydrogels: (a) the SEM images of the PVA hydrogel using sucrose as the template. The structure of the gel is highly porous and allows fast penetration of sweat; (b) the PVA hydrogel without sucrose template.

FIG. 34 shows BFC anode to cathode area ratio optimization: (a) CA of the BFCs at 0.4 V with different anode to cathode area ratio in 20 mM lactate environment; (b) bar graph summarizing the obtained power using different anode to cathode ratio, after 10 min of CA.

FIG. 35 shows LSV characterization of the cathode with different electrode materials. Pt deposited on planar screen-printed carbon electrode and CNT foam, as well as the p-Pt on the CNT foam are compared (scan rate: 5 mV s⁻¹).

FIG. 36 shows LSV characterization of the anode without and with 15 mM of lactate (scan rate: 5 mV s⁻¹).

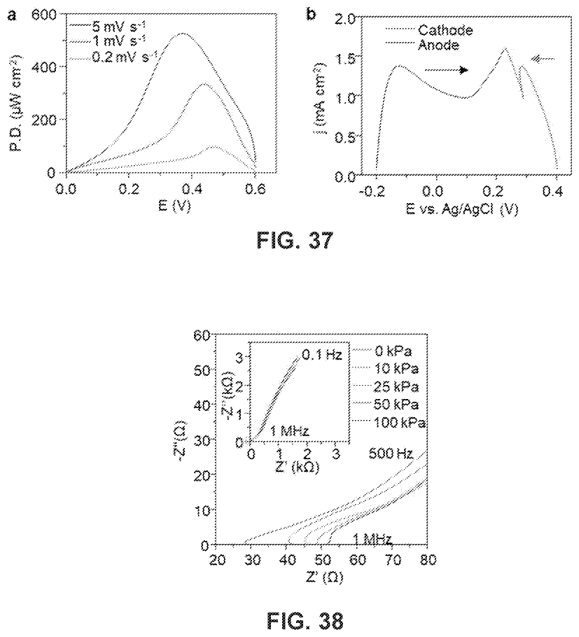

FIG. 37 shows LSV response of the BFC after area ratio optimization: (a) LSV power response of the BFC (1 cm²) with cathode and anode ratio of 1:2 in 0.5 M PBS with 20 mM of lactate at scan rate of 0.2, 1, and 5 mV s⁻¹; (b) potential vs. current density polarization curve during the 5 mV s¹ LSV measurement. The cathode potential started to decrease from 0.4 V to 0.23 V while the potential of anode increased from −0.2 V to 0.23 V.

FIG. 38 shows EIS Nyquist plot of the 2-electrode BFC covered by the porous PVA hydrogel with different applied pressure. The hydrogel is soaked in 0.1 M PBS prior to testing (scan range: 1 MHz-0.1 Hz; amplitude: 10 mV).

FIG. 39 shows optical microscopic images of the finger with applied bromophenol dye. Bromophenol green as a sweat indicator which is initially colorless and turns blue at above pH 5.4 is used for sweat rate analysis. All subjects washed hands before experiment and used their index finger to monitor the sweat rate and the microscope picture is taken until 10 min. The density of blue dots indicates the sweat rate difference on each subject.

FIG. 40 shows BFC performance with subjects with different natural fingertip sweat rates: (a) power obtained from different subjects with different sweat rate using CA at 0.4 V; (b) bar plot representing total harvested energy for 30 min from different subjects.

FIG. 41 shows hydrogel stability in extended harvesting tests. The BFC is covered by the PVA hydrogel and pressed for 3 min and rested for 1 hour. Without rehydrating the hydrogel after 1 hour, the BFC is pressed again for 3 min. The hydrogel is not able to retain the water without encapsulation, and the electrodes lost connection after 90 min.

FIG. 42 shows repeated pressing of the BFC. One BFC device is pressed repeatedly by one finger for 30 s every 5 min. The power generated by the BFC after touching increased from ˜15 μW to ˜40 μW via repeated refuelling the device. Total energy harvested in 30 min is 67.7 mJ.

FIG. 43 shows energy harvesting from low-intensity desk work. The BFC is wrapped around the right index finger of a subject (different from that of FIG. 24 (h)(ii)) for an hour. The subject is asked to perform normal work such as typing, clicking the mouse, or writing. The graph records the power of BFC during 1 hour of such activity while discharged at 0.4 V. In total, 35.3 mJ of energy is harvested within 1 hour.

FIG. 44 shows energy harvesting from no activity during overnight sleeping: (a) the power harvested by wrapping one BFC around the index finger of a subject (same subject as FIG. 24 (f)(iii)) is measured over 10 h of sleep. The total amount of energy harvested is 364.4 mJ; (b) the power harvested by wrapping one BFC around the index finger of a subject with lower sweat rate (different subject as in a) is measured over 6.5 h of sleep. The total amount of energy harvested is 253.0 mJ.

FIG. 45 shows power harvested from the BFC that is pressed by finger with different sweat generation time. The subject's finger is washed and dried thoroughly, and waited for (a) 1 min, (b) 3 min, (c) 5 min, and (d) 10 min, for before pressing the BFC device for 30 s. Although the energy harvested in the first 5 min is similar, the amount of energy harvested in 30 min showed more difference. Pressure applied, 50 kPa; discharge voltage, 0.4 V.

FIG. 46 shows power of the BFC pressed with different frequencies. The BFC is pressed with different frequencies for 5 min while maintaining 60% of contact time, including (a) 0.5 bpm (72 s pressing, 48 s release), (b) 1 bpm (36 s pressing, 24 s release), (c) 3 bpm (12 s pressing, 8 s release), (d) 4 bpm (9 s pressing, 6 s release), (e) 6 bpm (6 s pressing, 4 s release), and (f) 12 bpm (3 s pressing, 2 s release).

FIG. 47 shows OCV of the PZT chips pressed with different pressure at the centre. The 1×2 cm² PZT chip is pressed with (a) 10 kPa, (b) 25 kPa, (c) 50 kPa, and (d) 100 kPa d on the centre (1 cm² area, corresponding to the BFC) with 0.5 mm high spacer on two sides on its back.

FIG. 48 shows the energy harvesting using the PZT chip with different operation conditions. The 1×2 cm² PZT chip is pressed on the centre with (a) different height of spacer (0.1, 0.5 and 1 mm), (b) pressure (10 kPa, 25 kPa, 50 kPa, and 100 kPa), and (c) frequency (3 bpm, 4 bpm, 6 bpm and 12 bpm). Pressing with 100 kPa and 1 mm high spacer showed fastest charging speed compared to other conditions, however, applying 100 kPa can potentially damage the PVA hydrogel, whereas 1 mm spacer can lead to cracking on the PZT chip. Thus, 50 kPa pressure and spacer height of 0.5 mm is determined to be optimal for subsequent experiments.

FIG. 49 shows charging the capacitor using the integrated device with subjects with different sweat rates. The integrated harvester (with two BFC and two PZT chips) powered by two subjects with different sweat rate (different from FIG. 26 (h)(i) pressing the system to charge a 100 μF capacitor from 2 V-4 V repeatedly.

FIG. 50 shows a system flow chart of the integrated system and corresponding voltage values.

FIG. 51 shows schematics of the integrated circuit board: (a) circuit layout for the AtTiny441 MCU; (b) circuit layout for the bg25505 booster, analogue switch, and the bridge rectifier.

FIG. 52 shows MCU power consumption at different operation voltages.

FIG. 53 shows a capacitor charge flow to MCU. A 220 μF capacitor is charged to different voltages and discharged to the MCU. As shown, there is no significant benefit from increasing the voltage of the capacitor to the runtime of the system.

FIG. 54 shows MCU output voltage and charge to ECD: (a) the voltage of different capacitor discharged from 4 V; (b) the amount of charge available from the MCU to the display from capacitors charged to 4 V with different capacitance.

FIG. 55 shows an example of layer-by-layer printing and assembly of the ECD panel.

FIG. 56 shows photographic images of the printed ECD displaying different contents.

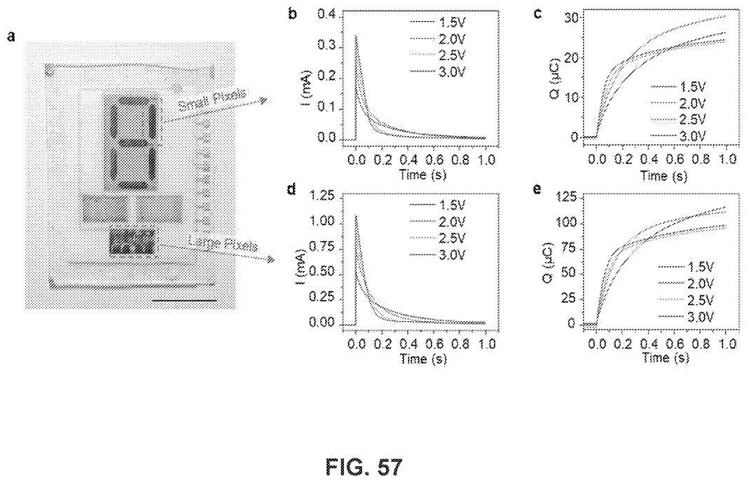

FIG. 57 shows current and charge consumption of the printed ECD: (a) photographic image displaying two sizes of pixels on the panel, including the 7 smaller pixels on top and the 3 larger rectangular pixels on the bottom; (b)-(c) the turn-on current b and charge c required for the smaller pixels at different voltages; (d)-(e) the turn-on current d and charge e required for the smaller pixels at different voltages.

FIG. 58 shows an example of layer-by-layer printing and drop-casting of the sensors: (a) printing and drop-casting of the Na+ sensor; (b) printing and drop-casting of the vitamin C sensor.

FIG. 59 shows vitamin C sensor calibration; (a) potentiometric signal obtained in 0.1 MPBS pH 7.4, upon spiking vitamin C concentrations from 200 to 1000 μM while discharged with a load of 10 MΩ; (b) calibration curve and exponential fitting curve (n=3). RSD=1.05%.

FIG. 60 shows an optimization of the vitamin C sensor: (a) the voltage of the sensor before and after pressed by the finger with different sweat accumulation time (10, 30, 60, 120, 180 and 300 s) before touching the sensor. Optimal waiting time of 1 min is determined; (b) the voltage of the sensor before and after pressed by the finger with different pressing time (10, 30, 60, 120, 180 and 300 s). Optimal pressing time of 2 min is determined; (c) control test using a covered finger. The sensor showed no response upon the pressure applied by the finger.

FIG. 61 shows vitamin C determination in sweat from fingertip for 2 subjects. Potentiometric response is measured after 20, 60 and 120 min of intaking a 1,000 mg vitamin C pill. A fresh hydrogel is used for each measurement: (a) subject 1, (b) subject 2.

Example Fabrication Techniques

Flexible Carbon Foam Characterization

(1) Fabrication of the Flexible CNT Foam

To synthesize the flexible, water-wicking CNT foam, a mixture of carboxylated multiwalled carbon nanotubes (MWCNT-COOH, CNT) and graphite, as the conductive carbon fillers, sodium bicarbonate (NaHCO₃) particles as the template, and styrene-ethyl butylene-styrene block copolymer (SEBS) as the elastomeric binder, are mixed into a paste using a toluene solvent. As shown in FIG. 28 (a), 0.25 g of the CNT and 0.05 g of graphite are added to a glass vial followed by adding 6.0 g of NaHCO₃. Then, 3.0 g of SEBS resin (4 g SEBS dissolved in 10 ml toluene) is carefully added to previous mixed powder and mixed well at 1800 rotations per minute (rpm) for 5 min. Then, 2.5 ml of toluene is further added to previous mixture and mixed again at 1800 rpm for 5 min. The composite CNT paste is then then ready for subsequent use.

To fabricate the CNT foam (FIG. 28 (b)), the composite paste is casted into stencil with desired thickness and size. In this work, glass slides with thicknesses of 1 mm are used as the stencil to control the thickness of the foam. Immediately after casting, the deposited paste is immersed in ethanol for 20 min to toluene-ethanol solvent exchange, which solidify the SEBS and prevent the collapse of the foam structure during drying process. After solvent exchange, carbon foam is naturally dried at room temperature without any heating process. To dissolve all the NaHCO₃ while making the CNT form hydrophilic, the CNT foam is soaked in 0.1 M hydrochloric acid (HCl) for 5 h and washed with distilled water several times to remove acid residue from the carbon foam. Finally, the carbon foam is dried at 80° C. oven and kept in ambient condition. The fabricated CNT foam is flexible, hydrophilic and exhibit good water permeability and able to wick water, as shown in FIGS. 29-30.

(2) Modification and Optimization of the BFC

To functionalize the CNT foam as biofuel cell (BFC) electrodes, the foam is firstly cut into 1×0.3 cm² pieces, and glued onto a prepared silver current collector as shown in FIG. 31. The porous platinum (Pt) electrode (p-Pt CF) is fabricated using co-electrodeposition of copper (Cu) and Pt onto carbon-foam electrode at −0.75 V, followed by electrochemical etching (dealloying) of the Cu using cyclic voltammetry between 0 V to 1.5 V at 50 mV s⁻¹ for 40 cycles. The resultant p-Pt CF is highly porous and deposited throughout the 3D CNT foam, as shown in the scanning electron spectroscopy (SEM) images with electron dispersive X-ray spectroscopy (EDS) on FIG. 32. The resulting electrodes clearly demonstrate advantages of the 3D p-Pt CF structure over Pt-SPC and Pt CF in terms of onset reduction potential, and current density originated from the O₂ electrocatalytic reduction on Pt, as evidenced by the linear sweep voltammetry (LSV) of FIG. 35. Whereas carbon foam-based Pt cathodes showed decent cathodic current stemming from an onset potential of 0.3 V, the p-Pt cathode displayed a higher oxygen reduction reaction (ORR) onset potential originating from 0.4 V (vs. Ag/AgCl) and a higher current density over the operating potential range of the cathode. The anode is fabricated by decorating the carbon foam with 1,4-naphthoquinone (NQ), LOx, and chitosan, to ensure efficient electron mediation and a uniform LOx surface coverage. The anode is also characterized using LSV, and displayed an increase of anodic current, with the onset potential of −0.2 V (vs. Ag/AgCl), upon increasing the lactate concentration from 0 to 15 mM (FIG. 36).

As shown from above single-electrode characterization, it is observed that the current from the cathode is significantly higher compared to the anode. In order to ensure the maximized utilization of both the anode and cathode in a limited area, the area ratio between the anode and cathode is optimized. As shown in FIG. 34, the system, discharged under 0.4 V using chronoamperometry (CA), is mostly limited by the anode. Thus, the anode and cathode of 0.6 cm²: 0.3 cm² selected to maximize the power in a limited area.

The assembled BFC with the 2:1 ratio is tested using linear scan voltammetry (LSV) under different scan rates, which also showed a large discrepancy of power that varied between ca. 500 μW/cm² at 5 mV s⁻¹ and ca. 100 μW cm⁻² at 0.2 mV s⁻¹, due to the large double layer charging current on the highly porous electrodes (FIG. 37). Thus, to accurate measure the power of the BFC, CA is preferred over LSV to remove the effect of the high charging current. The individual electrode potential shift during the 2-electrode LSV is also observed using and external reference electrode, which shows that the power-limiting anode potential shifted from −0.2V to +0.23V vs, as opposed to the cathode that shifted from +0.4 V to +0.23 V only.

All above in-vitro test are performed in 0.5 mM phosphate buffer solution (PBS) at pH of 7.4.

Sweat Rate Study

To analyse the performance of the touch-based BFC on different subjects with different passive sweat rates on the finger, the sweat rate of individuals are qualitatively compared. To estimate qualitatively of the sweat rate, impressions of the sweat glands is obtained using bromophenol green as the sweat indicator. Bromophenol green is initially colourless, and at pH >5.4 a blue coloration can be observed. As the sweat pH lies between 5-7, bromophenol can be used to visualize the number of sweat glands and the amount of sweat.

A 5 wt % solution of bromocresol green is prepared by dissolving in silicon oil and sonicated for 20 minutes. The oil is applied to the index finger of three subjects after thoroughly washing and drying the hands, and microscopic optical images are taken up to 10 minutes. As shown in FIG. 39, the 3 subjects exhibited different sweat rate in the first 10 min, with subject 1 exhibiting the most number and area of coloration on the figure, followed by subject 3, with subject 2 exhibiting the least amount of sweating.

As the amount of fuel and its lactate concentration determines the power of the touch-basedBFC, the power of the BFC is tested with all 3 subjects with different sweat rates by pressing their finger on the BFC for 30 s, followed by 30 min of resting. The power and the amount of energy collected within 30 min is shown in FIG. 40, which shows that the sweat rate is positively correlated with the power and energy harvested from the BFC, with subject 1 giving the most energy of 12 mJ, followed by subject 3 for 7 mJ and subject 2 for 5.5 mJ.

Example Design of the Power-Management, Sensing, and Display Control Circuit

The integrated circuit is designed to regulate and store the harvested energy from the BFC and the PZT chips, and use the stored energy to power a microcontroller that record signal from the sensor and display the sensing result on the electrochromic display (ECD). The design of the circuit is modified based on previous work. To regulate the power of the BFC, a voltage booster is used, which increase the low voltage of the BFC (0-0.6 V) to 2-5.5 V. The integrated energy management function in the booster allows programable maximum voltage (V_{BAT_OK_HYST}) and minimum voltage (V_{BAT_OK}) allowed for the connected energy storage device. A digital output from the booster turns on when the voltage of the connected capacitor increases above V_{BAT_OK_HYST} and turns off when the voltage drops below V_{BAT_OK}, which is used to control an analog switch that controls the connection of the capacitor to the microcontroller. Abridge rectifier is used to rectify the alternating input from the PZT generator, and the regulated output is connected to the capacitor to store the harvested energy. The circuit diagram is shown in FIG. 51.

The power consumption of the microcontroller is characterized by supplying a constant potential (FIG. 52), with the power varies from 6 mW-16 mW depending on the voltage applied. The charge required for the ECD is also characterized, which requires at least 150 μC and 100 ms of operation time regardless of the voltage used. Thus, to maximize the efficiency of the system, lower operation voltage is preferred as less charge is used for the MCU. Simultaneously, as the lower voltage charging requires lower quality energy, as shown in FIG. 4h, charging a 220 μF capacity from 2 V to 3 V takes less time than charging a 100 μF capacitor from 2 V to 4 V, while being able to store more charge for the ECD colour change. Thus, a system storage capacitor of 220 μF is selected, which charges and discharges in the window between 2 V and 3 V is used, giving a theoretical charge of 220 μC, with ˜150 μC available for ECD.

Fabrication and Characterization of the Example Electrochromic Display

(1) Modification and Optimization of the ECD

The substrate for the ECD is composed of styrene ethylene butylene styrene triblock copolymer (SEBS), and is fabricated by doctor blade casting (500 μm thick) of a resin of the SEBS dissolved in toluene (40 wt %) followed by curing in oven at 80° C. for 1 hour.

The ECD is fabricated using layer-by-layer screen-printing with customized inks. The ink formulation is adapted from a previous work. The printing of the ECD relies on four inks: the electrochromic poly(3,4-ethylenedioxythiophene) polystyrene sulfonate (PEDOT:PSS) ink, the silver ink for interconnection, the opaque insulator ink composed of SEBS and TiO₂, and the sodium polystyrene sulfonate-based electrolyte ink. The PEDOT:PSS ink is composed of PEDOT:PSS paste, toluene, deionized (DI) water, sodium dodecylbenzene sulfonate (DBSS), and fluorosurfactantFS-65 in 10:1.7:1.5:0.1:0.14 weight ratio. The silver ink is composed of silver flake, SEBS, and toluene in 1:0.16:0.5 weight ratio. The opaque insulator ink is composed of TiO₂, SEBS, and toluene in 1:6:10 weigh ratio. The PSSNa electrolyte ink is formulated by mixing PSSNa, D-sorbitol, glycerol, TiO₂, and poly acrylamide (PAM) precursor solution in 4:1:1:0.8:2 weight ratio. The PAM precursor is formulated by mixing acrylamide, DI water, potassium peroxydisulfate, and N,N′-methylenebisacrylamide in 1:10:0.05:0.01 ratio. All inks are mixed in the planetary mixer at 2500 rpm for 10 min or until homogenous.

The ECD panel is composed of the colour-charging front panel and the back panel to control the regional colour change. The layer-by-layer printing steps are shown in FIG. 55. Shortly, the PEDOT:PSS ink is firstly printed onto the SEBS substrate, and cured in the oven at 100° C. for 2 h. The silver interconnection and the opaque insulator layers are printed, with each layer cured at 80° C. for 10 min. Before assembly, the electrolyte is printed onto the back panel and briefly heated in the oven for 15 s at 80° C. to cross-link the PAM in the electrolyte. The front panel is then aligned and covered onto the bottom panel. Lastly, the device is heat sealed on all four sides to finish the device assembly.

(2) Characterization of the ECD

The colour change of the PEDOT:PSS relies on the redox reaction between two electrodes, with the reduced PEDOT:PSS showing the colour of darkblue (on), and the oxidized PEDOT:PSS showing translucent blue colour (off). By design, the 7 pixel segments on the top of the panel can display 1 digit of number, and the 3 larger pixels on the bottom showing the ×0.1 and ×10 multipliers, and the unit of mM. Combining the 10 pixels, 30 level of concentration can be displayed using the printed ECD panel (FIG. 56), as well as the letter “L” when the concentration is below the sensing range, or “H” when the concentration is above the sensing range.

As the voltage and the amount of charge available from the capacitor is limited, the minimum voltage and the charge required for the colour changing of the ECD is characterized in order to maximize the system efficiency. As the charge required of the ECD is mostly determined by the electrode area, the charge required and the turn-on behaviour of the ECD should be characterized differently for the top 7 smaller pixels and the bottom 3 pixels. As shown in FIG. 57, both pixels changes colour above 1.5 V, with the turn-on time below 100 ms. The smaller pixels consume 10-20 μC per pixel to change colour, and the larger pixels consumes 50-100 μC per pixel. In total, the minimum of charge required to change the display content is 30-150 μC, depending on the numbers of pixels to turn “on” needed.

Fabrication, characterization, and optimization of the example sensors

(1) Fabrication of the sensors

The Na⁺ sensors and vitamin C sensors are screen printed and modified via drop-casting. A silver ink, a carbon ink, the SEBS resin, and an Ag₂O ink. The formulation of the SEBS resin and the silver ink is described in this patent document. The formulation of the carbon ink is adapted from a previous work: graphite, super-P carbon black, SEBS, and toluene is added in a 6:1:3.4:6 weight ratio. The formulation of the Ag₂O ink is adapted from a previous work: super-P carbon black, Ag₂O, SEBS and toluene is mixed in a 0.05:0.95:0.18:0.82 weight ratio. Both inks are mixed in a planetary mixer at 2500 rpm for 10 min or until homogenous prior to printing. After printing each layer, the inks are cured in the oven at 80° C. for 10 min. The printing and modification of both sensors is shown in FIG. 58. The formulations and protocol for drop-casting are described in the Method section in the main text.

(2) Characterization and Optimization of the Vitamin C Sensor

The vitamin C analytical performance is studied in vitro, where the sensors presented high reproducibility (RSD=1.05%) (FIG. 59) and selectivity (FIG. 27F). An exponential calibration curve based on is obtained for programming the MCU to convert the voltage from the sensor to the display content (Table 3). To optimize the use of the sensor for sensing the natural sweat from the fingertip, the sweat accumulation time and the touching time is optimized based on a previously reported work. The sweat accumulation time is optimized by thoroughly cleaning the index finger and waited for different amount of time (10, 30, 60, 120, 180, and 300 s) before pressing the sensor that is covered by a small piece of the porous PVA hydrogel for 3 min. The pressing time on the sensor is optimized by thoroughly cleaning the finger followed by pressing the sensor for different amount of time (10, 30, 60, 120, 180, and 300 s). After touching, the voltage of the sensor under 10 MΩ load is recorded. As shown on FIG. 60 (a)-(b), the optimal waiting time is determined to be 1 min before the concentration changes further, and the optimal pressing time is determined to be 2 min. To confirm the effect of voltage change is resulted by the content transferred from the natural finger sweat, the sensor is also touch by a covered finger, which resulted in no voltage change in the sensor (FIG. 60 (c)).

Using the optimized accumulation and pressing time, the sensor is tested with two subjects for the determination of vitamin C concentration in natural finger sweat. The subjects are asked to take a 1,000 mg of vitamin C supplement, with the voltage signal is measured 20, 60 and 120 min after the vitamin intake. A fresh sensor is used in each trial. (FIG. 61). The voltage shows an increase after 20 min of taking the pill, and slowly dropped afterwards for over 2 h.

For each on-body measurement, 40 μL of 0.1 M PBS is added to a small PVA hydrogel that is firstly pat dry with paper to remain the electrolyte gel weight constant. All on body experiments are performed in strict compliance with IRB approved by UCSD.

TABLE 1
Table 1: Comparison of the energy return of investment
(ROI) of various wearable bioenergy harvesters.

| | | | Max. | Max. active |
|---|---|---|---|---|
| Type of | Average Power | Power | Energy | time in a |
| harvester | input* | harvested* | ROI*, ** | day(%)* |
| Biofuel cell | Running/Cycling | 10⁻⁵-10⁻³ | 0.001% | 5% |
| (exercise) | 10²-10³ W | W cm⁻² | | |
| Piezoelectric/ | Finger moving | 10⁻¹⁰-10⁻⁷ | 1% | 10% |
| triboelectric | (fast) | W cm⁻² | | |
| generator | 10⁻³-10⁻⁴ W | | | |
| | Hand moving | 10⁻⁶-10⁻⁴ | 0.1% | 5% |
| | 0.1-1 W | W cm⁻² | | |
| | Arm moving | 10⁻⁶-10⁻⁴ | 0.01% | 10% |
| | 1-10 W | W cm⁻² | | |
| | Walking | 10⁻⁶-10⁻² | 0.01% | 10% |
| | 50-100 W | W cm⁻² | | |
| | Running | | | |
| | 100-1000 W | | | |
| Biofuel cell | Finger | 10⁻⁵ | 1000%-∞ | 100% |
| (natural sweat) | contact/pressing | W cm⁻² | | |
| (this work) | (slow) | | | |
| | 0-10⁻⁵ W | | | |

Here, numbers are accurate to order of magnitude, and it is assumed device area of 10¹ cm².

TABLE 2
Table 2: Na⁺ sensor voltage to display content conversion.

| | |
|---|---|
| Sensor Potential | Display content |

| <15 | mV | L | |
|---|---|---|---|
| 15-45 | mV | 1 × 0.1 | mM |
| 45-59 | mV | 2 × 0.1 | mM |
| 59-68 | mV | 3 × 0.1 | mM |
| 68-75 | mV | 4 × 0.1 | mM |
| 75-80 | mV | 5 × 0.1 | mM |
| 80-85 | mV | 6 × 0.1 | mM |
| 85-88 | mV | 7 × 0.1 | mM |
| 88-92 | mV | 8 × 0.1 | mM |
| 92-95 | mV | 9 × 0.1 | mM |
| 95-107 | mV | 1 | mM |
| 107-121 | mV | 2 | mM |
| 121-130 | mV | 3 | mM |
| 130-137 | mV | 4 | mM |
| 137-142 | mV | 5 | mM |
| 142-147 | mV | 6 | mM |
| 147-151 | mV | 7 | mM |
| 151-154 | mV | 8 | mM |
| 154-157 | mV | 9 | mM |
| 157-170 | mV | 1 × 10 | mM |
| 170-184 | mV | 2 × 10 | mM |
| 184-193 | mV | 3 × 10 | mM |
| 193-199 | mV | 4 × 10 | mM |
| 199-205 | mV | 5 × 10 | mM |
| 205-209 | mV | 6 × 10 | mM |
| 209-213 | mV | 7 × 10 | mM |
| 213-216 | mV | 8 × 10 | mM |
| 213-220 | mV | 9 × 10 | mM |

| | | |
|---|---|---|
| >220 | mV | H |

TABLE 3
Table 3: Vitamin C sensor voltage to display content conversion.

| Sensor Potential | Display content |
|---|---|
| <171 mV | L |
| 171-201 mV | 1 × 0.1 mM |
| 201-224 mV | 2 × 0.1 mM |
| 224-241 mV | 3 × 0.1 mM |
| 241-255 mV | 4 × 0.1 mM |
| 255-266 mV | 5 × 0.1 mM |
| 266-274 mV | 6 × 0.1 mM |
| 274-280 mV | 7 × 0.1 mM |
| 280-285 mV | 8 × 0.1 mM |
| 285-289 mV | 9 × 0.1 mM |
| >289 mV | H |

Monitoring Parkinson Disease Therapy Using Touch-Based L-Dopa Sweat Sensor

While Levodopa is considered to be extremely effective treatment of Parkinson's Disease (PD) the high variability in levodopa plasma concentrations with oral levodopa-carbidopa treatment often results in sub-optimal efficacy, particularly during the progress of PD.

Orally-administered levodopa (1-dopa) is regarded as the “platinum” standard of PD therapeutics for its impact on disability and discomfort and its cost-effectiveness.

Large and inconsistent fluctuations in plasma concentrations cause difficulty with the long-term management of PD patients with conventional levodopa formulations.

Following administration of levodopa/carbidopa microtablets

The aim is to investigate the pharmacokinetic profiles of levodopa and carbidopa, and to assess motor function following a single-dose microtablet administration in Parkinson's disease patients.

With the flexibility that the microtablets provide, the individualization of treatment may become easier, with respect to fine-tuned dosing.

Levodopa (L-Dopa) is the ‘gold-standard’ medication toward symptomatic therapy of Parkinson disease (PD) patients. However, its long-term use is associated with the onset of motor and non-motor complications, mostly due to its fluctuating plasma levels. The disclosed technology can be implemented in some embodiments to provide an individualized therapeutic drug monitoring for PD patients upon intake of standard oral pill formulations, centered on dynamic tracking of L-Dopa levels in naturally secreted thermoregulatory sweat. The detection method relies on instantaneous collection of fingertip sweat on a porous hydrogel (via touching a porous hydrogel on the electrode surface) which mediates the sweat transport to a tyrosinase enzyme-modified electrode, where sweat L-Dopa is indirectly measured via following reduction current of the dopaquinone enzymatic product. Individualized response to L-Dopa pill intake is demonstrated within a small group of healthy human subjects, along with the pharmacokinetic correlation of finger touch-based sweat and capillary blood samples. This non-invasive detection method holds considerable promise toward realizing patient-specific dose regulation and optimal therapeutic outcomes towards individualized treatment involving fine-tuned L-Dopa dosing.

Parkinson disease (PD) is a chronic, progressive neurodegenerative disease affecting more than 6 million individuals worldwide. levodopa (L-Dopa), the precursor of dopamine, is the most effective drug for management of PD and is considered the gold standard treatment. However, the long-term administration of oral L-Dopais associated with the onset of motor and non-motor complications, stemming mainly from fluctuations in the plasma L-Dopa level. L-Dopa has a narrow therapeutic window, as suboptimal dosing causes the patients to remain stiff, slow, and have tremors while overdosing generates excessive, involuntary movements. Therapeutic window becomes narrower with the disease progression, which makes the patients to take higher doses at more frequent intervals. Another complication is the high interpatient variability in the response to L-Dopa therapy which requires a patient-specific dosing regimen. Such inconsistent large fluctuations in the plasma drug concentrations hamper the management of PD patients and leads to sub-optimal therapeutic efficacy, particularly during the disease progress. Therefore, a device capable of rapidly monitoring the level of L-Dopa near or on-the-patient is highly advantages toward L-Dopa dose regulation and thus avoiding the motor fluctuations of PD patients. Nevertheless, there is no device available to continuously monitor the individualized therapeutic levels of L-Dopa. The current ‘gold standard’ method to detect plasma L-Dopa levels relies on liquid chromatography-mass spectroscopy (LC-MS) technique performed in centralized laboratories, which due to the invasiveness, long turn-around times and the need for specialized instrument and skilled personnel, it cannot be adopted for clinical practice and its use has been limited to rare occasions of limited pharmacokinetic studies. Thus, mobile, decentralized, and wearable electrochemical sensors in the form of strips, microneedles, and sweat band have been proposed to address this challenge. While such electrochemical platforms offer potential for frequent monitoring of L-Dopa, they are mainly limited to in-vitro demonstrations and the singular case of in-vivo study of sweatband has been reported in connection to uptake of fava beans (not the standard pill formulations) which makes its application for realistic therapeutic monitoring of PD patients unclear. Not involved the standard pill formulations XXX Additionally, the large amount of proteins found in beans greatly limits the L-Dopa absorption from the gastrointestinal tract to the circulation system due to the fact that L-Dopa shares a similar absorption mechanism with the dietary amino acids.

The disclosed technology can be implemented in some embodiments to provide an individualized therapeutic drug monitoring for PD patients, centered on dynamic non-invasive tracking pharmacokinetic profiles of L-Dopa levels in the secreted sweat upon intake of standard pill formulations. Sweat is a non-invasively retrievable biofluid containing rich information of trace-level, health-related biochemical markers. Wearable sweat sensors have shown enormous potential toward monitoring of physiological heath status (e.g., hydration), disease diagnosis and management (e.g., diabetes and gout), and therapeutic drug monitoring (e.g., pain management). However, the presence of skin as a mechanical barrier prevents an uninterrupted access to this information-rich biofluid, and thus a triggering system (i.e., physical exercise, thermal stimulation, oriontophoresis) is necessary to provide continuous access to sweat sample. In contrast to such vigorous active stimulation methods, natural perspiration route has demonstrated immense potential to realize simple, easy, and continuous access to the sweat fluid for chemical analysis. Taking advantage of the high density of eccrine sweat glands (˜400 glands cm²) and the consequent generation of high sweat rates, finger touch-based biosensors have recently been reported for the detection of key sweat biomarkers (e.g., glucose, vitamin C, and cortisol). Leveraging such natural thermoregulatory sweat sample, a finger-touch L-Dopa biosensor based on some embodiments of the disclosed technology can continuously monitor the dynamic profile of sweat L-Dopa upon intake of standard anti-Parkinsonian medication including L-Dopa-carbidopa (100:25 mg) (FIG. 5A(a)). The current signal difference measured in 10-minute intervals shows a rise in sweat L-Dopa signal shortly after the intake of medication, reaching its peak level, after which the signal declined to its background level (FIG. 5A(b)). Interestingly, the signal validation of the obtained sweat samples performed versus capillary blood samples showed a similar pharmacokinetic profile with negligible (˜10 min) lag time. The analysis of L-Dopa simply relied on touching a porous hydrogel on the electrode surface with index finger to allow fast transfer of the natural sweat to the electrode modified with the immobilized tyrosinase enzyme (FIG. 5B), upon which sweat L-Dopa is oxidized to dopaquinone via its reaction with the immobilized tyrosinase enzyme. The enzymatically generated dopaquinone is electrochemically reduced back to L-Dopa at the applied potential of −0.3 V, with the resulted amperometric signal correlated with the dynamically fluctuating level of L-Dopa. Such non-invasive, fast, and simple touch-based procedure holds considerable promise toward guiding dose adjustments in PD patients via capturing real-time fluctuations in the sweat L-Dopa levels.

towards individualized treatment involving fine-tuned L-Dopa dosing for PTM of other drugs and management of other diseases

Referring back to FIGS. 5A-5B, the disclosed technology can be implemented in some embodiments to provide a fingertip L-Dopa biosensor. FIG. 5A (a) is schematic illustration of the finger touch-based procedure before and after intake of the anti-Parkinsonian medication and FIG. 5A (b) is the typical current-time profile recorded every 10 min. FIG. 5B (a) is schematic depiction of the underlying mechanism of L-Dopa detection, starting with (a) touching the sensor with index finger, (b) transfer of natural sweat containing L-Dopa from the skin surface through the porous hydrogel to the electrode surface, where it is electrochemically measured at tyrosinase immobilized electrode.

Referring back to FIGS. 6A-6C, the disclosed technology can be implemented in some embodiments to provide L-Dopa monitoring using the touch-based sensor. FIG. 6A shows time course of a cycle of L-Dopa detection in fingertip sweat, including measuring current before touching (2 min), touching (2 min), measurement after touching (2 min), and waiting for the next cycle (4 min). FIG. 6B shows dynamic pharmacokinetic profile during intake of an L-Dopa/C-Dopa pill by the subject. FIG. 6C shows the corresponding chronoamperograms obtained from time −10 min to +60 min (three initial amperograms of −40, −30, and −20 min are not shown for clarity), where the black and red curves represent before and after touching, respectively.

L-Dopa detection is achieved through coupling the tyrosinase enzyme-catalyzed L-Dopa oxidation (catecholase activity) and the subsequent electrochemical reduction of the corresponding quinone product, dopaquinone, at low potentials (FIG. 6A). The formed reaction cycle not only enhances the sensitivity through amplification of the resulting current signal but also prevents electrode fouling by inhibiting the spontaneous polymerization reactions of the unstable quinone molecules. Tyrosinase enzyme is simply immobilized on the surface of screen-printed carbon electrodes, followed by crosslinking with glutaraldehyde to prevent leaching of the enzyme (FIG. 6A). To establish the optimal potential for the L-Dopaquinone reduction, the amperometric signals obtained at various potentials, ranging from −0.1 to −0.4 V (vs. internal pseudo-reference Ag/AgCl), are compared upon addition of 10 μM L-Dopa. The current response dramatically increased upon decreasing potential from −0.1 to −0.3 V, after which the signal declined probably due to the interference from oxygen reduction reaction. Thus, optimal potential of −0.3 V is chosen and used for subsequent in-vitro and on-body experiments. FIG. 6A shows the resulted amperometric responses of L-Dopa addition from 5 to 30 μM, giving well-defined linearity within the whole physiological concentration range. The reproducibility of the L-Dopa sensor is evaluated by measuring 10 μM L-Dopa concentration on six different electrodes sensors. The results revealed high fabrication reproducibility of the sensor with a relative standard deviation (RSD) of 2.6%. Moreover, the carry-over experiment is performed to evaluate the repeatability of the sensor using 0 and 15 μM of L-Dopa for multiple times (n=5) which showed negligible drift on the signal.

The performance of the sensor toward following the L-Dopa pharmacokinetics is characterized on healthy patients following the administration of L-Dopa/C-Dopa (100:25 mg) pills which are common oral medication for PD patients. C-Dopais an amino acid (dopa) decarboxylase enzyme inhibitor and is combined with L-Dopato enhance the bioavailability of the drug. C-Dopa is an o-diphenolic compound and can be oxidized by the tyrosinase enzyme, and thus may interfere with the target L-Dopa detection. The selectivity of the sensor is challenged via detecting L-Dopa/C-Dopa in 4:1 concentration ratio, similar to the pill composition. The overall response obtained from C-Dopa showed ˜20% when the same concentration is used as L-Dopa, while only ˜6% of current is observed when a quarter of concentration is applied which reflects relative amount in the medication. These selectivity test indicate minimal interference of C-Dopa as desired for accurate and reliable L-Dopa detection. Atypical measurements of the target L-Dopa following the pill intake are carried out at 10 min intervals. FIG. 6A depicts the optimal time course of a single 10-min cycle of on-body L-Dopa sensing protocol, including an initial 2-min recording of the background current on a buffer solution-soaked porous hydrogel coated electrode (without fingertip touch) by XXX, followed by placing the index finger on the gel (covering the working electrode) for 2 min, during which sweat diffuses to the electrode surface, and subsequently stepping the potential to −0.3V recording the current signal for 2 min. Following each cycle, the subject is asked to wait for 4 min before starting the next cycle. FIG. 6B displays a typical peak-shaped dynamic profile of the subject sweat L-Dopa levels over the 100 min test period, involving 5 and 6 measurements before and after taking the pill, respectively. The corresponding raw current signals and backgrounds are shown in FIG. 6C. These data of FIGS. 6B and 6C show negligible changes prior to the pill intake, with the L-Dopa current signal starts to increase 10 min after pill intake, reaching its peak maximum at time 30 min, after which signal decreases back to its background level nearly one hour after taking the pill. These results clearly demonstrate that the touch-based L-Dopa sensor can successfully tracking variation of L-Dopa sweat level. As will be shown below, such peak-shaped temporal profile matches closely the corresponding blood L-Dopa concentration (with a short ˜10-min time delay).

Referring back to FIG. 7, personalized pharmacokinetic profiles of L-Dopa drug in three different healthy subjects are shown: (A) chronoamperograms obtained every 10 min for three subjects (a-c), with black and red curves showing before and after touching current measurements; (B) the relevant temporal current profiles for three subjects. The blue dotted line in each diagram indicates the time of pill intake.

To gain further insight into the personalized body responses upon medication intake, the performance of the finger touch biosensor is evaluated on three different individuals while taking the same medication under identically kept conditions (FIG. 7 (a)-(c)). The subjects are asked not to eat any food or drink caffeinated liquids for two hours before the experiment to exclude any unwanted interference from the dietary proteins or from caffein. The same protocol as in FIG. 6A is followed for sweat collection and signal recording in each subject. Interestingly, all three subjects showed similar temporal profile of sweat L-Dopa level, reaching their peak maximum 30 min after pill intake. Additionally, the different peak currents are observed within three subjects, where the highest and lowest signals are obtained for the first (a) and third (c) individual, respectively. While this may partly be attributed to different sweat secretion rates of the subjects, it also implies the large interindividual variability in pharmacokinetic profiles upon L-Dopa drug intake.

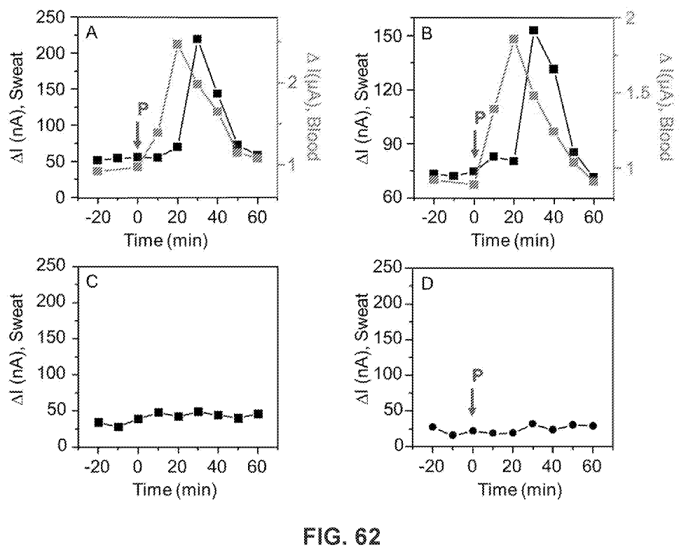

FIG. 62 shows pharmacokinetic correlation of response to L-Dopa using natural sweat and capillary blood samples: (A)-(B) continuous monitoring of sweat (black) and blood (red) L-Dopa every 10 min in different subjects; (C)-(D) the results of control experiments performed without pill consumption (C) and using electrode without enzyme modification (D). The letter “P” indicates the time of pill intake.

While the blood plasma is the ‘gold standard’ matrix for therapeutic monitoring of L-Dopa, this analysis method relies on LC-MS centralized instruments. To further confirm the reliability of the developed protocol based on touch-based sweat L-Dopa detection, the feasibility of data validation between sweat and blood samples is investigated. Two subjects performed fingertip sweat sensing in parallel to the electrochemical measurements of the finger pricked capillary blood samples, using the enzymatic L-Dopa sensor. As shown in FIG. 62 (A)-(B), the sweat sensor is able to probe and detect successfully L-Dopa fluctuations in microliter blood samples similar to the fingertip natural sweat sample. Similar temporal profiles are observed for blood and sweat experiments, with ˜10 min lag time. Blood rising XX after taking the pill, compared to XX This first demonstration of pharmacokinetic correlation for sweat and blood L-Dopa profiles suggests the significant potential of the developed procedure toward tracking dynamic pharmacokinetic behavior of PD patients in a non-invasive and continuous manner. Additionally, control experiments are also carried out without pill intake (FIG. 62 (C)) and using non-enzymatic electrode (FIG. 62 (D)). In both control experiments, the current signal remained small and unchanged, which indicates the signal specificity of the fingertip touch-based sweat sensing procedure.

As such, non-invasive sweat measurements offer considerable potential for tracking the pharmacokinetic profiles of L-Dopa following a single-dose microtablet administration.

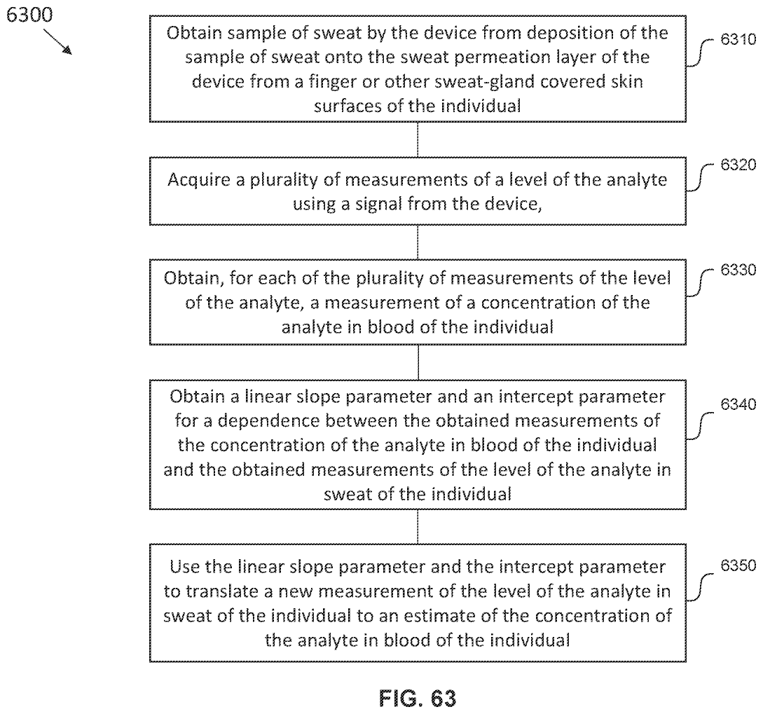

FIG. 63 shows an example method 6300 for determining a concentration of an analyte in at least one of blood, sweat, or interstitial fluid (ISF) of an individual based on some embodiments of the disclosed technology.

In some implementations, the method 6300 includes, at 6310, obtaining sample of sweat by the device disclosed in this patent document from deposition of the sample of sweat onto the sweat permeation layer of the device from a finger or other sweat-gland covered skin surfaces of the individual, at 6320, acquiring a plurality of measurements of a level of the analyte using a signal from the device disclosed in this patent document, at 6330, obtaining, for each of the plurality of measurements of the level of the analyte, a measurement of a concentration of the analyte in blood of the individual, at 6340, obtaining a linear slope parameter and an intercept parameter for a dependence between the obtained measurements of the concentration of the analyte in blood of the individual and the obtained measurements of the level of the analyte in sweat of the individual, and, at 6350, using the linear slope parameter and the intercept parameter to translate a new measurement of the level of the analyte in sweat of the individual to an estimate of the concentration of the analyte in blood of the individual.

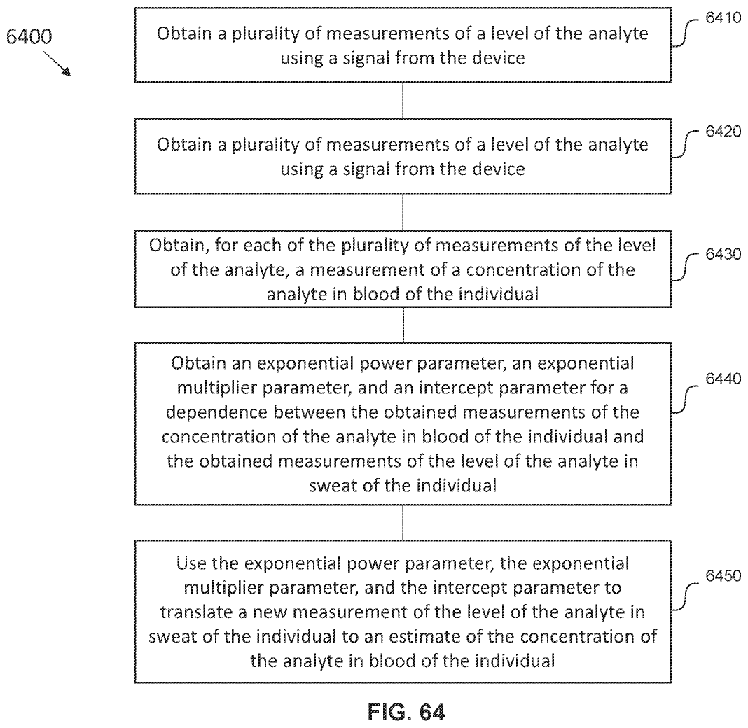

FIG. 64 shows an example method 6400 for determining a concentration of an analyte in at least one of blood, sweat, or interstitial fluid (ISF) of an individual based on some embodiments of the disclosed technology.

In some implementations, the method 6400 includes, at 6410, obtaining sample of sweat by the device disclosed in this patent document from deposition of the sample of sweat onto the sweat permeation layer of the device from a finger of the individual, at 6420, acquiring a plurality of measurements of a level of the analyte using a signal from the device disclosed in this patent document, at 6430, obtaining, for each of the plurality of measurements of the level of the analyte, a measurement of a concentration of the analyte in blood of the individual, at 6440, obtaining an exponential power parameter, an exponential multiplier parameter, and an intercept parameter for a dependence between the obtained measurements of the concentration of the analyte in blood of the individual and the obtained measurements of the level of the analyte in sweat of the individual, and, at 6450, using the exponential power parameter, the exponential multiplier parameter, and the intercept parameter to translate a new measurement of the level of the analyte in sweat of the individual to an estimate of the concentration of the analyte in blood of the individual.

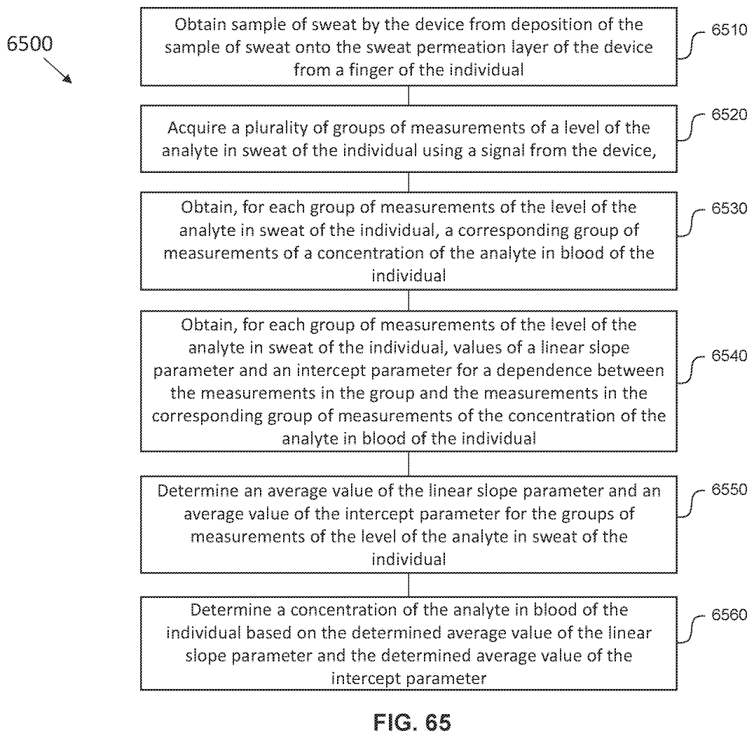

FIG. 65 shows an example method 6500 for determining a concentration of an analyte in blood of an individual based on some embodiments of the disclosed technology.

In some implementations, the method 6500 includes, at 6510, obtaining sample of sweat by the device disclosed in this patent document from deposition of the sample of sweat onto the sweat permeation layer of the device from a finger of the individual, at 6520, acquiring a plurality of groups of measurements of a level of the analyte in sweat of the individual using a signal from the device disclosed in this patent document, wherein the sweat is collected by the device from a finger of the individual in contact with the sweat permeation layer of the device, at 6530, obtaining, for each group of measurements of the level of the analyte in sweat of the individual, a corresponding group of measurements of a concentration of the analyte in blood of the individual, at 654030, obtaining, for each group of measurements of the level of the analyte in sweat of the individual, values of a linear slope parameter and an intercept parameter for a dependence between the measurements in the group and the measurements in the corresponding group of measurements of the concentration of the analyte in blood of the individual, at 6550, determining an average value of the linear slope parameter and an average value of the intercept parameter for the groups of measurements of the level of the analyte in sweat of the individual, and, at 6560, determining a concentration of the analyte in blood of the individual based on the determined average value of the linear slope parameter and the determined average value of the intercept parameter.

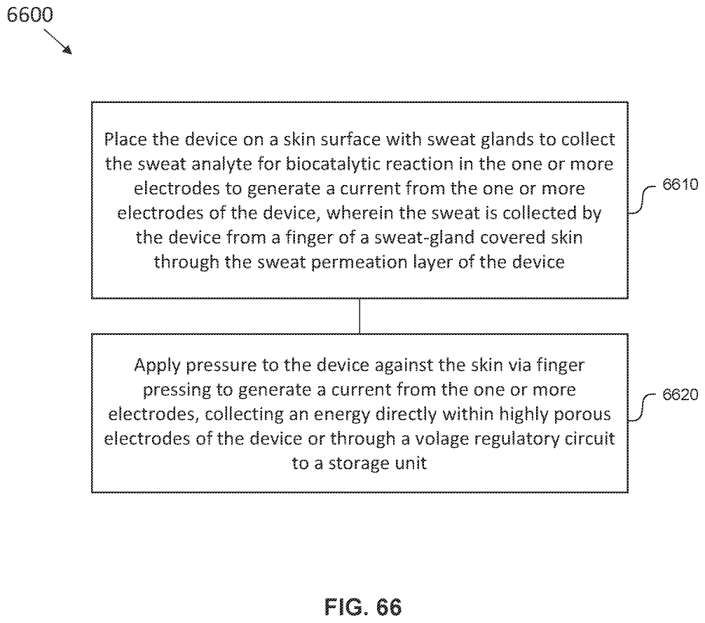

FIG. 66 shows an example method 6600 for generating power using a sweat analyte based on some embodiments of the disclosed technology.

In some implementations, the method 6600 includes, at 6610, placing the device on a skin surface with sweat glands to collect the sweat analyte for biocatalytic reaction in the plurality of electrodes to generate a current from the plurality of electrodes of the device disclosed in this patent document, wherein the sweat is collected by the device from a finger of a sweat-gland covered skin through the sweat permeation layer of the device, and, at 6620, applying pressure to the device against the skin via finger pressing to generate a current from the plurality of electrodes, collecting an energy directly within highly porous electrodes of the device or through a voltage regulatory circuit to a storage unit.

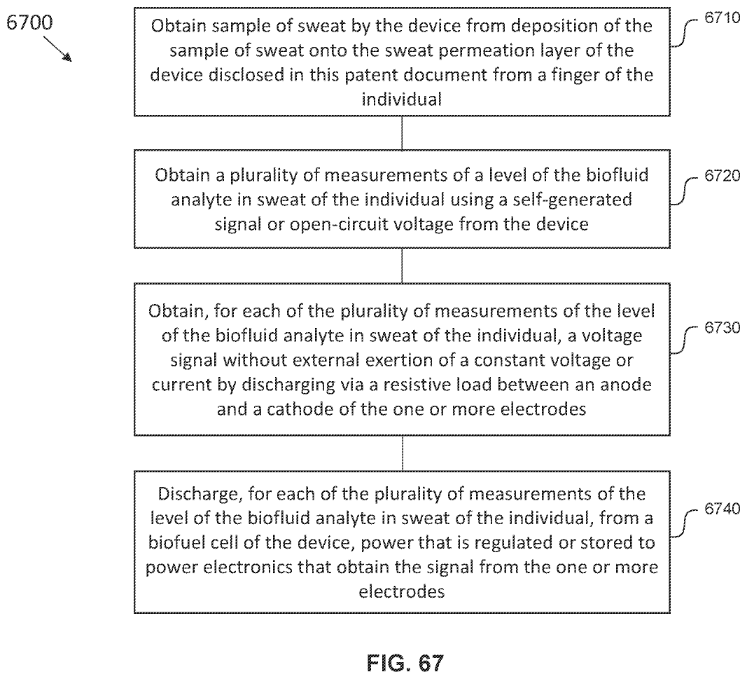

FIG. 67 shows an example method 6700 for determining a concentration of a biofluid analyte of an individual based on some embodiments of the disclosed technology.

In some implementations, the method 6700 includes, at 6710, obtaining sample of sweat by the device from deposition of the sample of sweat onto the sweat permeation layer of the device disclosed in this patent document from a finger of the individual, at 6720, acquiring a plurality of measurements of a level of the biofluid analyte in sweat of the individual using a self-generated signal or open-circuit voltage from the device, at 6730, obtaining, for each of the plurality of measurements of the level of the biofluid analyte in sweat of the individual, a voltage signal without external exertion of a constant voltage or current by discharging via a resistive load between an anode and a cathode of the plurality of electrodes, and, at 6740, discharging, for each of the plurality of measurements of the level of the biofluid analyte in sweat of the individual, from a biofuel cell of the device, power that is regulated or stored to power electronics that obtain the signal from the plurality of electrodes.

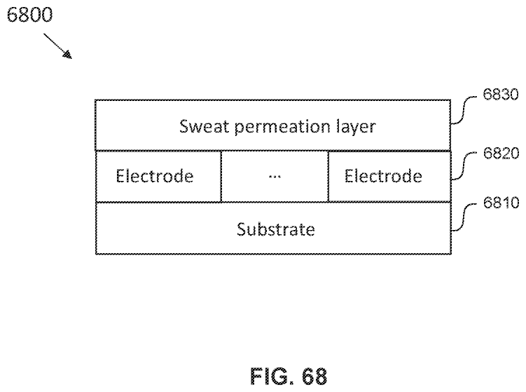

FIG. 68 shows an example of a device 6800 for collecting sweat for the estimation of a concentration of a blood analyte or the utilization of the redox reaction of the analyte for energy generation based on some embodiments of the disclosed technology.

Referring to FIG. 68, the device 6800 may include a substrate 6810, a plurality of electrodes 6820 disposed on the substrate 6810 and operable to detect an analyte in sweat of an individual, and a sweat permeation layer 6830 including a hydrogel and having a first side and a second side located opposite to the first side, wherein the first side of the sweat permeation layer is in contact with the plurality of electrodes 6820 such that the plurality of electrodes 6820 is disposed between the substrate and the first side of the sweat permeation layer, wherein the sweat permeation layer allows the analyte in sweat applied to the second side to permeate through the sweat permeation layer to reach the plurality of electrodes 6820 through the first side of the sweat permeation layer 6830.

Therefore, various implementations of features of the disclosed technology can be made based on the above disclosure, including the examples listed below.

### Examples 1-47

Example 1. A device for sweat-based estimation of a concentration of a blood analyte, comprising: a substrate; a sensor disposed on the substrate and operable to detect an analyte in sweat; and a sweat permeation layer having a first side and a second side located opposite to the first side, wherein the first side of the sweat permeation layer is in contact with the sensor such that the sensor is disposed between the substrate and the first side of the sweat permeation layer, and wherein the sweat permeation layer is structured to allow sweat applied to the second side permeate through the sweat permeation layer to reach the sensor through the first side of the sweat permeation layer.

Example 2. The device of example 1, wherein the sensor is one of: an electrochemical sensor, an affinity-based sensor, or an optical sensor.

Example 3. The device of example 1, wherein the sweat permeation layer includes a layer of a hydrogel.

Example 4. The device of example 3, wherein the hydrogel includes one of: polyvinyl alcohol (PVA), agarose, or glycerol.

Example 5. The device of example 1, wherein the analyte is glucose, and the sensor includes an electrochemical sensor comprising a reference electrode, a working electrode, and a counter electrode, wherein the reference electrode includes silver, and wherein the working electrode includes Prussian blue and glucose oxidase.

Example 6. The device of example 1, comprising a processor and a memory, wherein the memory stores instructions which, when executed by the processor, cause the processor to convert an output signal from the sensor corresponding to a concentration of the analyte in the sweat into a numeric value corresponding to a concentration of the analyte in blood.

Example 7. A method of determining a concentration of a blood analyte, comprising: obtaining, for an individual, several measurements of a level of the analyte in sweat of the individual using a signal from the sensor of the device according to any of examples 1-6, wherein the sweat is collected by the device from a finger of the individual touching the sweat permeation layer of the device; for each measurement in the several measurements of the level of the analyte in the sweat of the individual, obtaining a measurement of a concentration of the analyte in blood of the individual; obtaining a linear slope parameter and an intercept parameter for a dependence between the obtained measurements of the concentration of the analyte in the blood of the individual and the obtained measurements of the level of the analyte in the sweat of the individual; and using the linear slope parameter and the intercept parameter to translate a new measurement of the level of the analyte in the sweat of the individual to an estimate of the concentration of the analyte in the blood of the individual.

Example 8. A method of determining a concentration of a blood analyte, comprising: obtaining, for an individual, several groups of measurements of a level of the analyte in sweat of the individual using a signal from the sensor of the device according to any of examples 1-6, wherein the sweat is collected by the device from a finger of the individual in contact with the sweat permeation layer of the device; for each group of measurements of the level of the analyte in the sweat of the individual, obtaining a corresponding group of measurements of a concentration of the analyte in blood of the individual; for each group of measurements of the level of the analyte in the sweat of the individual, obtaining values of a linear slope parameter and an intercept parameter for a dependence between the measurements in the group and the measurements in the corresponding group of measurements of the concentration of the analyte in the blood of the individual; determining an average value of the linear slope parameter and an average value of the intercept parameterfor the groups of measurements of the level of the analyte in the sweat of the individual; and using the determined average value of the linear slope parameter and the determined average value of the intercept parameter to determine a concentration of the analyte in the blood of the individual using a measurement of the level of the analyte in the sweat of the individual provided by the device.

Example 9. A sweat-collection device for estimation of a concentration of an analyte in blood of an individual or for utilization of a redox reaction of the analyte for energy generation, comprising: a substrate; one or more electrodes disposed on the substrate and operable to detect the analyte in sweat and/or perform energy harvesting from the analyte in sweat; and a sweat permeation layer having a first side and a second side located opposite to the first side, wherein the first side of the sweat permeation layer is in contact with the one or more electrodes such that the one or more electrodes are disposed between the substrate and the first side of the sweat permeation layer, and wherein the sweat permeation layer is structured to allow sweat applied to the second side permeate through the sweat permeation layer to reach the one or more electrodes through the first side of the sweat permeation layer.

Example 10. The device of example 9, wherein the one or more electrodes are a part of one of: an electrochemical sensor, an affinity-based sensor, an optical sensor, a catalytic fuel cell, or a biocatalytic fuel cell.

Example 11. The device of example 9, wherein the sweat permeation layer comprises a layer of a hydrogel.

Example 12. The device of example 11, wherein the hydrogel includes at least one of: polyvinyl alcohol (PVA), poly acrylic acid (PAA), poly methyl methacrylate (PMMA), polyethylene oxide (PEO), polyacrylamide (PAM), a cellulosic material, agar, gelatin, agarose, alginate, glycerol, ethylene carbonate, or propylene carbonate.

Example 13. The device of example 12, wherein the cellulosic material is one of: cellulose, methylcellulose, ethylcellulose, carboxymethyl cellulose, or hydroxyethylcellulose.

Example 14. The device as in any of examples 11-13, wherein the hydrogel is disposable after each use of the device.

Example 15. The device as in any of examples 11-13, wherein the hydrogel is reusable.

Example 16. The device of example 15, further comprising a container or a compartment configured for placement of the hydrogel into the container or the compartment, storage of the hydrogel in the container or the compartment and retrieval of the hydrogel from the container or the compartment.

Example 17. The device as in any of examples 9-16, wherein the analyte is glucose, and the one or more electrodes form an electrochemical sensor comprising a reference electrode, a working electrode, and a counter electrode, wherein the reference electrode includes silver, and wherein the working electrode includes Prussian blue and glucose oxidase.

Example 18. The device as in any of examples 9-16, wherein the analyte is lactate, and the one or more electrodes include an electrocatalytic anode and a cathode, wherein the cathode includes one of: a catalyst that is configured to facilitate an oxygen reduction reaction including at least one of: platinum, carbon black, carbon nanotubes, bilirubin oxidase, laccase, platinum-cobalt alloy, platinum-iron alloy, platinum-gold alloy, platinum-nickel alloy, or an oxidative material that can be reduced, including one of: silver oxide, nickel oxide, or manganese oxide, and wherein the anode includes lactate oxidase and a reaction mediator.

Example 19. The device of example 18, wherein the reaction mediator is one of: tetrathiafulvalene (TTF), naphthoquinone (NQ), ferrocene, or a derivative of ferrocene.

Example 20. The device of example 19, wherein the derivative of ferrocene is one of: methylferrocene or dimethylferrocene.

Example 21. The device of example 18, wherein the reaction mediator is a complex of tetrathiafulvalene (TTF), naphthoquinone (NQ), ferrocene, or a derivative of ferrocene with one or more chemical compounds.

Example 22. The device of example 21, wherein the reaction mediator is tetrathiafulvalene tetracyanoquinodimethane.

Example 23. The device of example 9, wherein an electrode in the one or more electrodes includes a carbonaceous material, an elastomeric binder, and a redox reaction active material, and wherein the electrode is structured to have a degree of porosity created by adding and subsequently removing template particles from the electrode during its production.

Example 24. The device of example 23, wherein the carbonaceous material includes one of: graphite, carbon black, carbon nanotubes, or graphene.

Example 25. The device of example 23, wherein the elastomeric binder includes one of: a styrene-based triblock copolymer, a fluorinated rubber, polyethylene vinyl acetate, polyurethane, Ecoflex, or Poly dimethylsiloxane.

Example 26. The device of example 25, wherein the styrene-based triblock copolymer is one of: polystyrene-polyisoprene-poly styrene or poly styrene-polybutylene-polyethylene-polystyrene.

Example 27. The device of example 25, wherein the fluorinated rubber is poly (vinylfluoride-tetrafluoropropylene).

Example 28. The device of example 23, wherein the template particles include one of: a salt, sucrose, a metal, or a polymer.

Example 29. The device of example 28, wherein the salt is one of: sodium chloride or sodium bicarbonate.

Example 30. The device of example 28, wherein the metal is one of: Mg or Zn.

Example 31. The device of example 28, wherein the polymer is styrene.

Example 32. The device of example 23, wherein the redox reaction active material includes one of: a conductive polymer, a 2-D material, or a MXene.

Example 33. The device of example 32, wherein the conductive polymer is poly(3,4-ethylenedioxythiophene) polystyrene sulfonate.

Example 34. The device of example 32, wherein the 2-D material is molybdenum disulfide.

Example 35. The device of example 32, wherein the MXeneis Ti2C3.

Example 36. The device of example 9, wherein an electrode in the one or more electrodes includes a conductive polymer, a redox-active material that is co-deposited onto the electrode with the conductive polymer and wherein the electrode is structured to have one or more recognition cavities that are structured to selectively bind with the analyte.

Example 37. The device of example 36, wherein the conductive polymer is one of: polypyrrole, polyethylenimine, or polyaniline.

Example 38. The device of example 36, wherein the redox-active material includes a mediator or an organic dye.

Example 39. The device of example 9, comprising a voltage regulatory circuit coupled to at least an electrode of the one or more electrodes and configured to harvest electric energy generated by the device and store that energy in an energy storage device.

Example 40. The device of example 39, wherein the energy storage device is one of: a capacitor, a supercapacitor, a battery, or a combination thereof.

Example 41. A method of generating power using a collected sweat analyte, comprising: placing the device as in any of the examples 9-40 on a sweat-gland covered skin area to collect the analyte for a biocatalytic reaction in the one or more electrodes of the device to generate a current from the one or more electrodes of device, wherein the sweat is collected by the device from the sweat-gland covered skin area through the sweat permeation layer of the device; collecting the generated current directly or through a voltage regulatory circuit to a storage unit; and discharging the storage unit.

Example 42. The method of example 41 further comprising: applying pressure to the device against the skin area using a finger.

Example 43. The method of example 42, wherein the pressure application is performed in a sporadic or a periodic manner.

Example 44. The method of example 41 wherein the storage unit is an electrode of the device.

Example 45. A method of determining a concentration of an analyte in blood of an individual, comprising: obtaining, for the individual, several measurements of a level of the analyte in sweat of the individual using a signal from the device according any of the examples 9-39, wherein the sweat is collected by the device from a finger of the individual touching the sweat permeation layer of the device; for each measurement in the several measurements of the level of the analyte in the sweat of the individual, obtaining a measurement of a concentration of the analyte in the blood of the individual; obtaining an exponential power parameter, and exponential multiplier parameter, and an intercept parameter for a dependence between the obtained measurements of the concentration of the analyte in the blood of the individual and the obtained measurements of the level of the analyte in the sweat of the individual; and using the exponential power parameter, exponential multiplier parameter, and the intercept parameter to translate a new measurement of the level of the analyte in the sweat of the individual to an estimate of the concentration of the analyte in the blood of the individual.

Example 46. Methods, systems and devices as described in this patent document.

Example 47. Any combination of the above examples.

Examples A1-A51

In some embodiments in accordance with the present technology (example A1), a device includes a substrate; a plurality of electrodes disposed on the substrate and operable to detect an analyte in sweat of an individual; and a sweat permeation layer including a hydrogel and having a first side and a second side located opposite to the first side, wherein the first side of the sweat permeation layer is in contact with the plurality of electrodes such that the plurality of electrodes is disposed between the substrate and the first side of the sweat permeation layer, wherein the sweat permeation layer is configured to transfer the sweat containing the analyte that is naturally produced from the individual's fingertip by permeating the naturally produced sweat through the sweat permeation layer from the second side to the first side to reach the plurality of electrodes.

Example A2 includes the device of any of examples A1-A37, further comprising a processor configured to estimate a concentration of the analyte in blood of the individual by comparing the concentration of the analyte in sweat with a concentration of the analyte in blood measured by a reference device.

Example A3 includes the device of example A2 or any of examples A1-A37, further comprising: a memory configured to store instructions which, when executed by the processor, cause the processor to convert an output signal from the device corresponding to the concentration of the analyte in sweat into a numeric value corresponding to a concentration of the analyte in blood.

Example A4 includes the device of any of examples A1-A37, further comprising a voltage regulatory circuit including: a voltage generator coupled to the plurality of electrodes to produce electricity by using a redox reaction of the analyte in sweat; and an energy storage device coupled to the voltage generator to store the generated electricity.

Example A5 includes the device of example A4 or any of examples A1-A37, wherein the voltage regulatory circuit increases a voltage, when connected to the plurality of electrodes, to cause an input signal from the plurality of electrodes to increase and be stored in an energy storage device.

Example A6 includes the device of any of examples A1-A37, wherein the plurality of electrodes are a part of one of: an electrochemical sensor, an affinity-based sensor, an optical sensor, a catalytic fuel cell, or a biocatalytic fuel cell.

Example A7 includes the device of any of examples A1-A37, wherein the hydrogel includes at least one of: polyvinyl alcohol (PVA), poly acrylic acid (PAA), poly methyl methacrylate (PMMA), polyethylene oxide (PEO), polyacrylamide (PAM), a cellulosic material, agar, gelatin, agarose, alginate, glycerol, ethylene carbonate, or propylene carbonate.

Example A8 includes the device of example A7 or any of examples A1-A37, wherein the hydrogel is structured to have a plurality of pores having a pore diameter of at least 50 nm that inhibits the flow of bulk fluid.

Example A9 includes the device of example A8 or any of examples A1-A37, wherein the hydrogel is created by adding and subsequently removing template particles from the hydrogel after crosslinking.

Example A10 includes the device of example A7 or any of examples A1-A37, wherein the cellulosic material includes at least one of cellulose, methylcellulose, ethylcellulose, carboxymethyl cellulose, or hydroxyethylcellulose.

Example A11 includes the device of any of examples A7-A10 or any of examples A1-A37, wherein the hydrogel is disposable after each use of the device.

Example A12 includes the device of any of examples A7-A10 or any of examples A1-A37, wherein the hydrogel is crosslinked directly on the surface of the plurality of electrodes.

Example A13 includes the device of any of examples A7-A10 or any of examples A1-A37, wherein the hydrogel is reusable.

Example A14 includes the device of example A13 or any of examples A1-A37, further comprising a container configured for storage of the hydrogel in the container and retrieval of the hydrogel from the container.

Example A15 includes the device of any of examples A1-A37, wherein the analyte is glucose, and the plurality of electrodes form an electrochemical sensor comprising a reference electrode, a working electrode, and a counter electrode, wherein the reference electrode includes silver, and wherein the working electrode includes Prussian blue and glucose oxidase.

Example A16 includes the device of any of examples A1-A37, wherein the analyte is lactate, and the plurality of electrodes include an electrocatalytic anode and a cathode, wherein the cathode includes at least one of: a catalyst that is configured to facilitate an oxygen reduction reaction including at least one of: platinum, carbon black, carbon nanotubes, bilirubin oxidase, laccase, platinum-cobalt alloy, platinum-iron alloy, platinum-gold alloy, platinum-nickel alloy, or an oxidative material capable of being reduced, including at least one of: silver oxide, nickel oxide, or manganese oxide, and wherein the anode includes lactate oxidase and a reaction mediator.

Example A17 includes the device of example A16 or any of examples A1-A37, wherein the reaction mediator includes at least one of tetrathiafulvalene (TTF), naphthoquinone (NQ), ferrocene, or a derivative of ferrocene.

Example A18 includes the device of example A17 or any of examples A1-A37, wherein the derivative of ferrocene includes at least one of methylferrocene or dimethylferrocene.

Example A19 includes the device of example A16 or any of examples A1-A37, wherein the reaction mediator includes tetrathiafulvalene tetracyanoquinodimethane.

Example A20 includes the device of any of examples A1-A37, wherein the plurality of electrodes includes a first electrode that includes a carbonaceous material, an elastomeric binder, and a redox reaction active material, and wherein the first electrode is structured to have a degree of porosity created by adding and subsequently removing template particles from the first electrode.

Example A21 includes the device of example A20 or any of examples A1-A37, wherein the carbonaceous material includes one of: graphite, carbon black, carbon nanotubes, or graphene.

Example A22 includes the device of example A20 or any of examples A1-A37, wherein the elastomeric binder includes at least one of a styrene-based triblock copolymer, a fluorinated rubber, polyethylene vinyl acetate, polyurethane, Ecoflex, or Polydimethylsiloxane.

Example A23 includes the device of example A22 or any of examples A1-A37, wherein the styrene-based triblock copolymer includes at least one of polystyrene-polyisoprene-polystyrene or polystyrene-polybutylene-polyethylene-polystyrene.

Example A24 includes the device of example A22 or any of examples A1-A37, wherein the fluorinated rubber includes poly (vinylfluoride-tetrafluoropropylene).

Example A25 includes the device of example A9 or example A20 or any of examples A1-A37, wherein the template particles include at least one of a salt, saccharide, a metal, or a polymer.

Example A26 includes the device of example A25 or any of examples A1-A37, wherein the salt includes at least one of sodium chloride or sodium bicarbonate.

Example A27 includes the device of example A25 or any of examples A1-A37, wherein the metal includes at least one of Mg or Zn.

Example A28 includes the device of example A25 or any of examples A1-A37, wherein the saccharide includes at least one of glucose, sucrose, fructose, maltodextrin, starch, or maltose.

Example A29 includes the device of example A25 or any of examples A1-A37, wherein the polymer includes polystyrene, polyethylene glycol, polyacrylamides, polyacrylic acid copolymer, polyethyleneimine, or polyvinyl alcohol.

Example A30 includes the device of example A20 or any of examples A1-A37, wherein the redox reaction active material includes one of: a conductive polymer, a 2-D material, or a MXene.

Example A31 includes the device of example A30 or any of examples A1-A37, wherein the conductive polymer includes poly(3,4-ethylenedioxythiophene) polystyrene sulfonate.

Example A32 includes the device of example A30 or any of examples A1-A37, wherein the 2-D material includes molybdenum disulfide.

Example A33 includes the device of example A30 or any of examples A1-A37, wherein the MXene includes Ti₂C₃, Ti₂C, V₂C, or Ti₄N₃.

Example A34 includes the device of any of examples A1-A37, wherein the plurality of electrodes includes a conductive polymer, a redox-active material, and a target analyte molecule of the device.

Example A35 includes the device of example A34 or any of examples A1-A37, wherein the conductive polymer includes at least one of polypyrrole, polyethylenimine, polyaniline, or poly(3,4-ethylenedioxythiophene) polystyrene sulfonate formed by direct dispersion deposition or applying a constantvoltage/current or a voltage range scanned repeatedly for a controlled amount of time.

Example A36 includes the device of example A34 or any of examples A1-A37, wherein the redox-active material includes a mediator or an organic dye that is co-deposited onto the one or more electrode during an electrodeposition of the conductive polymer.

Example A37 includes the device of example A34 or any of examples A1-A36, wherein the target analyte molecule includes at least one of cortisol, insulin, levodopa, or protein, wherein the plurality of electrodes includes a molecularly imprinted polymer electrode formed by applying a constant voltage, a voltage range scanned repeatedly, an aqueous solution, or an organic solution for a controlled amount of time such that the at least one of cortisol, insulin, levodopa, or protein is eluded from the plurality of electrodes, and wherein the molecularly imprinted polymer electrode includes recognition cavities that selectively bind with the analyte in sweat.

In some embodiments in accordance with the present technology (example A38), a device includes a piezoelectric chip; two or more electrodes including an anode electrode and a cathode electrode formed over the piezoelectric chip and operable to detect an electrical signal associated with a chemical reaction involving an analyte contained in sweat of an individual incident in a region at a surface of the anode electrode and the cathode electrode; a current collector including two or more electrically-conductive material structures disposed between the piezoelectric chip and the two or more electrodes to electrically couple at least one of the electrically-conductive material structures to the anode electrode and at least another one of the electrically-conductive material structures to the cathode electrode; and a sweat permeation layer including a hydrogel and having a first side and a second side located opposite to the first side, wherein the first side of the sweat permeation layer is in contact with the two or more electrodes and configured to transfer the sweat that is naturally produced from the individual's fingertip by permeating the naturally produced sweat through the sweat permeation layer from the second side to be pressed by the individual's fingertip to the first side to reach the region at the surface of the two or more electrodes, wherein the piezoelectric chip undergoes a non-destructive mechanical deformation upon pressing the second side of the sweat permeation layer with the individual's fingertip, generating electrical energy from the non-destructive mechanical deformation of the piezoelectric chip.

Example A39 includes the device of any of examples A37-A45, wherein the two or more electrodes are operable to measure a parameter of the analyte in the sweat based on the detected electrical signal.

Example A40 includes the device of any of examples A37-A45, further comprising: a substrate disposed under the piezoelectric chip; and two or more spacers disposed under the piezoelectric chip and above the substrate to have a first thickness that facilitates the non-destructive mechanical deformation of the piezoelectric chip.

Example A41 includes the device of any of examples A37-A45, wherein the hydrogel includes a porous polyvinyl alcohol (PVA) hydrogel.

Example A42 includes the device of any of examples A37-A45, wherein the two or more electrodes includes 3-dimensional (3D) carbon nanotube (CNT) foam.

Example A43 includes the device of example A42 or any of examples A37-A45, and the cathode electrode includes particles comprising platinum within pores or cavities in the 3D CNT foam of the cathode electrode.

Example A44 includes the device of example A43 or any of examples A37-A45, wherein the analyte includes lactate, and wherein the anode electrode includes lactate oxidase (LOx) within pores or cavities in the 3D CNT foam of the anode electrode.

Example A45 includes the device of example A44 or any of examples A37-A43, wherein the anode electrode further includes at least one of enzyme or mediator.

In some embodiments in accordance with the present technology (example A46), a method for determining a concentration of an analyte present in at least one of blood, sweat, or interstitial fluid (ISF) of an individual includes: obtaining a sample of sweat by the device according to any of claims 1-45 from deposition of the sample of sweat onto the sweat permeation layer of the device from a finger of the individual; acquiring a plurality of measurements of a level of the analyte using a signal from the device; obtaining, for each of the plurality of measurements of the level of the analyte, a measurement of a concentration of the analyte in blood of the individual; obtaining a linear slope parameter and an intercept parameter for a dependence between the obtained measurements of the concentration of the analyte in blood of the individual and the obtained measurements of the level of the analyte in sweat of the individual; and using the linear slope parameter and the intercept parameter to translate a new measurement of the level of the analyte in sweat of the individual to an estimate of the concentration of the analyte in blood of the individual.

In some embodiments in accordance with the present technology (example A47), a method for determining a concentration of an analyte present in at least one of blood, sweat, or interstitial fluid (ISF) of an individual includes obtaining a sample of sweat by the device according to any of claims 1-45 from deposition of the sample of sweat onto the sweat permeation layer of the device from a finger of the individual; acquiring a plurality of measurements of a level of the analyte using a signal from the device; obtaining, for each of the plurality of measurements of the level of the analyte, a measurement of a concentration of the analyte in blood of the individual; obtaining an exponential power parameter, an exponential multiplier parameter, and an intercept parameter for a dependence between the obtained measurements of the concentration of the analyte in blood of the individual and the obtained measurements of the level of the analyte in sweat of the individual; and using the exponential power parameter, the exponential multiplier parameter, and the intercept parameter to translate a new measurement of the level of the analyte in sweat of the individual to an estimate of the concentration of the analyte in blood of the individual.

In some embodiments in accordance with the present technology (example A48), a method for determining a concentration of an analyte present in blood of an individual includes obtaining a sample of sweat by the device according to any of claims 1-45 from deposition of the sample of sweat onto the sweat permeation layer of the device from a finger of the individual; acquiring a plurality of groups of measurements of a level of the analyte in sweat of the individual using a signal from the device; obtaining, for each group of measurements of the level of the analyte in sweat of the individual, a corresponding group of measurements of a concentration of the analyte in blood of the individual; obtaining, for each group of measurements of the level of the analyte in sweat of the individual, values of a linear slope parameter and an intercept parameter for a dependence between the measurements in the group and the measurements in the corresponding group of measurements of the concentration of the analyte in blood of the individual; determining an average value of the linear slope parameter and an average value of the intercept parameter for the groups of measurements of the level of the analyte in sweat of the individual; and determining a concentration of the analyte in blood of the individual based on the determined average value of the linear slope parameter and the determined average value of the intercept parameter.

In some embodiments in accordance with the present technology (example A49), a method for generating power using a sweat analyte includes placing the device on a skin surface with sweat glands to collect the sweat analyte for biocatalytic reaction in the plurality of electrodes to generate a current from the plurality of electrodes of the device according to any of claims 1-45, wherein the sweat is collected by the device from a finger of a sweat-gland covered skin through the sweat permeation layer of the device; and applying pressure to the device against the skin via finger pressing to generate a current from the plurality of electrodes, collecting an energy directly within highly porous electrodes of the device or through a voltage regulatory circuit to a storage unit.

In some embodiments in accordance with the present technology (example A50), a method for determining a concentration of a biofluid analyte of an individual includes obtaining a sample of sweat by the device according to any of claims 1-45 from deposition of the sample of sweat onto the sweat permeation layer of the device from a finger of the individual; acquiring a plurality of measurements of a level of the biofluid analyte in sweat of the individual using a self-generated signal or open-circuit voltage from the device; obtaining, for each of the plurality of measurements of the level of the biofluid analyte in sweat of the individual, a voltage signal without external exertion of a constant voltage or current by discharging via a resistive load between an anode and a cathode of the plurality of electrodes; and discharging, for each of the plurality of measurements of the level of the biofluid analyte in sweat of the individual, from a biofuel cell of the device, power that is regulated or stored to power electronics that obtain the signal from the plurality of electrodes.

Example A51 includes a method or device that includes any combination of the any of examples A1-A50.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A device, comprising:
a substrate;
a plurality of electrodes disposed on the substrate and operable to detect an analyte in sweat of an individual; and
a sweat permeation layer including a hydrogel and having a first side and a second side located opposite to the first side, wherein the first side of the sweat permeation layer is in contact with the plurality of electrodes such that the plurality of electrodes is disposed between the substrate and the first side of the sweat permeation layer,
wherein the sweat permeation layer is configured to transfer the sweat containing the analyte that is naturally produced from the individual's fingertip by permeating the naturally produced sweat through the sweat permeation layer from the second side to the first side to reach the plurality of electrodes.

2. The device of claim 1, further comprising:
a processor configured to estimate a concentration of the analyte in blood of the individual by comparing the concentration of the analyte in sweat with a concentration of the analyte in blood measured by a reference device.

3. The device of claim 2, further comprising:
a memory configured to store instructions which, when executed by the processor, cause the processor to convert an output signal from the device corresponding to the concentration of the analyte in sweat into a numeric value corresponding to a concentration of the analyte in blood.

4. The device of claim 1, further comprising:
a voltage regulatory circuit including: a voltage generator coupled to the plurality of electrodes to produce electricity by using a redox reaction of the analyte in sweat; and an energy storage device coupled to the voltage generator to store the generated electricity.

5. The device of claim 4, wherein the voltage regulatory circuit increases a voltage, when connected to the plurality of electrodes, to cause an input signal from the plurality of electrodes to increase and be stored in an energy storage device.

6. The device of claim 1, wherein the plurality of electrodes are a part of one of: an electrochemical sensor, an affinity-based sensor, an optical sensor, a catalytic fuel cell, or a biocatalytic fuel cell.

7. The device of claim 1, wherein the hydrogel includes at least one of: polyvinyl alcohol (PVA), poly acrylic acid (PAA), poly methyl methacrylate (PMMA), polyethylene oxide (PEO), polyacrylamide (PAM), a cellulosic material, agar, gelatin, agarose, alginate, glycerol, ethylene carbonate, or propylene carbonate.

8. The device of claim 7, wherein the hydrogel is structured to have a plurality of pores having a pore diameter of at least 50 nm that inhibits the flow of bulk fluid.

9. The device of claim 8, wherein the hydrogel is created by adding and subsequently removing template particles from the hydrogel after crosslinking.

10. The device of claim 7, wherein the cellulosic material includes at least one of cellulose, methylcellulose, ethylcellulose, carboxymethyl cellulose, or hydroxyethylcellulose.

11. The device of claim 7, wherein the hydrogel is disposable after each use of the device.

12. The device of claim 7, wherein the hydrogel is crosslinked directly on the surface of the plurality of electrodes.

13. The device of claim 7, wherein the hydrogel is reusable.

14. The device of claim 13, further comprising a container configured for storage of the hydrogel in the container and retrieval of the hydrogel from the container.

15. The device of claim 1, wherein the analyte is glucose, and the plurality of electrodes form an electrochemical sensor comprising a reference electrode, a working electrode, and a counter electrode, wherein the reference electrode includes silver, and wherein the working electrode includes Prussian blue and glucose oxidase.

16. The device of claim 1, wherein the analyte is lactate, and the plurality of electrodes include an electrocatalytic anode and a cathode, wherein the cathode includes at least one of: a catalyst that is configured to facilitate an oxygen reduction reaction including at least one of: platinum, carbon black, carbon nanotubes, bilirubin oxidase, laccase, platinum-cobalt alloy, platinum-iron alloy, platinum-gold alloy, platinum-nickel alloy, or an oxidative material capable of being reduced, including at least one of: silver oxide, nickel oxide, or manganese oxide, and wherein the anode includes lactate oxidase and a reaction mediator.

17. The device of claim 16, wherein the reaction mediator includes at least one of tetrathiafulvalene (TTF), naphthoquinone (NQ), ferrocene, or a derivative of ferrocene.

18. The device of claim 17, wherein the derivative of ferrocene includes at least one of methylferrocene or dimethylferrocene.

19. The device of claim 16, wherein the reaction mediator includes tetrathiafulvalene tetracyanoquinodimethane.

20. The device of claim 1, wherein the plurality of electrodes includes a first electrode that includes a carbonaceous material, an elastomeric binder, and a redox reaction active material, and wherein the first electrode is structured to have a degree of porosity created by adding and subsequently removing template particles from the first electrode.

21. The device of claim 20, wherein the carbonaceous material includes one of: graphite, carbon black, carbon nanotubes, or graphene.

22. The device of claim 20, wherein the elastomeric binder includes at least one of a styrene-based triblock copolymer, a fluorinated rubber, polyethylene vinyl acetate, polyurethane, Ecoflex, or Polydimethylsiloxane.

23. The device of claim 22, wherein the styrene-based triblock copolymer includes at least one of polystyrene-polyisoprene-polystyrene or polystyrene-polybutylene-polyethylene-polystyrene.

24. The device of claim 22, wherein the fluorinated rubber includes poly (vinylfluoride-tetrafluoropropylene).

25. The device of claim 20, wherein the template particles include at least one of a salt, saccharide, a metal, or a polymer.

26. The device of claim 25, wherein the salt includes at least one of sodium chloride or sodium bicarbonate.

27. The device of claim 25, wherein the metal includes at least one of Mg or Zn.

28. The device of claim 25, wherein the saccharide includes at least one of glucose, sucrose, fructose, maltodextrin, starch, or maltose.

29. The device of claim 25, wherein the polymer includes polystyrene, polyethylene glycol, polyacrylamides, polyacrylic acid copolymer, polyethyleneimine, or polyvinyl alcohol.

30. The device of claim 20, wherein the redox reaction active material includes one of: a conductive polymer, a 2-D material, or a MXene.

31. The device of claim 30, wherein the conductive polymer includes poly(3,4-ethylenedioxythiophene) polystyrene sulfonate.

32. The device of claim 30, wherein the 2-D material includes molybdenum disulfide.

33. The device of claim 30, wherein the MXene includes Ti₂C₃, Ti₂C, V₂C, or Ti₄N₃.

34. The device of claim 1, wherein the plurality of electrodes includes a conductive polymer, a redox-active material, and a target analyte molecule of the device.

35. The device of claim 34, wherein the conductive polymer includes at least one of polypyrrole, polyethylenimine, polyaniline, or poly(3,4-ethylenedioxythiophene) polystyrene sulfonate formed by direct dispersion deposition or applying a constant voltage/current or a voltage range scanned repeatedly for a controlled amount of time.

36. The device of claim 34, wherein the redox-active material includes a mediator or an organic dye that is co-deposited onto the one or more electrode during an electrodeposition of the conductive polymer.

37. The device of claim 34, wherein the target analyte molecule includes at least one of cortisol, insulin, levodopa, or protein, wherein the plurality of electrodes includes a molecularly imprinted polymer electrode formed by applying a constant voltage, a voltage range scanned repeatedly, an aqueous solution, or an organic solution for a controlled amount of time such that the at least one of cortisol, insulin, levodopa, or protein is eluded from the plurality of electrodes, and wherein the molecularly imprinted polymer electrode includes recognition cavities that selectively bind with the analyte in sweat.

38. A device, comprising:
a piezoelectric chip;
two or more electrodes including an anode electrode and a cathode electrode formed over the piezoelectric chip and operable to detect an electrical signal associated with a chemical reaction involving an analyte contained in sweat of an individual incident in a region at a surface of the anode electrode and the cathode electrode;
a current collector including two or more electrically-conductive material structures disposed between the piezoelectric chip and the two or more electrodes to electrically couple at least one of the electrically-conductive material structures to the anode electrode and at least another one of the electrically-conductive material structures to the cathode electrode; and
a sweat permeation layer including a hydrogel and having a first side and a second side located opposite to the first side, wherein the first side of the sweat permeation layer is in contact with the two or more electrodes and configured to transfer the sweat that is naturally produced from the individual's fingertip by permeating the naturally produced sweat through the sweat permeation layer from the second side to be pressed by the individual's fingertip to the first side to reach the region at the surface of the two or more electrodes,
wherein the piezoelectric chip undergoes a non-destructive mechanical deformation upon pressing the second side of the sweat permeation layer with the individual's fingertip, generating electrical energy from the non-destructive mechanical deformation of the piezoelectric chip.

39. The device of claim 38, wherein the two or more electrodes are operable to measure a parameter of the analyte in the sweat based on the detected electrical signal.

40. The device of claim 38, further comprising:
a substrate disposed under the piezoelectric chip; and
two or more spacers disposed under the piezoelectric chip and above the substrate to have a first thickness that facilitates the non-destructive mechanical deformation of the piezoelectric chip.

41. The device of claim 38, wherein the hydrogel includes a porous polyvinyl alcohol (PVA) hydrogel.

42. The device of claim 38, wherein the two or more electrodes includes 3-dimensional (3D) carbon nanotube (CNT) foam.

43. The device of claim 42, and the cathode electrode includes particles comprising platinum within pores or cavities in the 3D CNT foam of the cathode electrode.

44. The device of claim 43, wherein the analyte includes lactate, and wherein the anode electrode includes lactate oxidase (LOx) within pores or cavities in the 3D CNT foam of the anode electrode.

45. The device of claim 44, wherein the anode electrode further includes at least one of enzyme or mediator.

46. A method for determining a concentration of an analyte present in at least one of blood, sweat, or interstitial fluid (ISF) of an individual, comprising:
obtaining a sample of sweat by the device according to claim 1 from deposition of the sample of sweat onto the sweat permeation layer of the device from a finger of the individual;
acquiring a plurality of measurements of a level of the analyte using a signal from the device;
obtaining, for each of the plurality of measurements of the level of the analyte, a measurement of a concentration of the analyte in blood of the individual;
obtaining a linear slope parameter and an intercept parameter for a dependence between the obtained measurements of the concentration of the analyte in blood of the individual and the obtained measurements of the level of the analyte in sweat of the individual; and
using the linear slope parameter and the intercept parameter to translate a new measurement of the level of the analyte in sweat of the individual to an estimate of the concentration of the analyte in blood of the individual.

47. A method for determining a concentration of an analyte present in at least one of blood, sweat, or interstitial fluid (ISF) of an individual, comprising:
obtaining a sample of sweat by the device according to claim 1 from deposition of the sample of sweat onto the sweat permeation layer of the device from a finger of the individual;
acquiring a plurality of measurements of a level of the analyte using a signal from the device;
obtaining, for each of the plurality of measurements of the level of the analyte, a measurement of a concentration of the analyte in blood of the individual;
obtaining an exponential power parameter, an exponential multiplier parameter, and an intercept parameter for a dependence between the obtained measurements of the concentration of the analyte in blood of the individual and the obtained measurements of the level of the analyte in sweat of the individual; and
using the exponential power parameter, the exponential multiplier parameter, and the intercept parameter to translate a new measurement of the level of the analyte in sweat of the individual to an estimate of the concentration of the analyte in blood of the individual.

48. A method for determining a concentration of an analyte present in blood of an individual, comprising:
obtaining a sample of sweat by the device according to claim 1 from deposition of the sample of sweat onto the sweat permeation layer of the device from a finger of the individual;
acquiring a plurality of groups of measurements of a level of the analyte in sweat of the individual using a signal from the device;
obtaining, for each group of measurements of the level of the analyte in sweat of the individual, a corresponding group of measurements of a concentration of the analyte in blood of the individual;
obtaining, for each group of measurements of the level of the analyte in sweat of the individual, values of a linear slope parameter and an intercept parameter for a dependence between the measurements in the group and the measurements in the corresponding group of measurements of the concentration of the analyte in blood of the individual;
determining an average value of the linear slope parameter and an average value of the intercept parameter for the groups of measurements of the level of the analyte in sweat of the individual; and
determining a concentration of the analyte in blood of the individual based on the determined average value of the linear slope parameter and the determined average value of the intercept parameter.

49. A method for generating power using a sweat analyte, comprising:
placing the device on a skin surface with sweat glands to collect the sweat analyte for biocatalytic reaction in the plurality of electrodes to generate a current from the plurality of electrodes of the device according to claim 1, wherein the sweat is collected by the device from a finger of a sweat-gland covered skin through the sweat permeation layer of the device; and
applying pressure to the device against the skin via finger pressing to generate a current from the plurality of electrodes, collecting an energy directly within highly porous electrodes of the device or through a voltage regulatory circuit to a storage unit.

50. A method for determining a concentration of a biofluid analyte of an individual, comprising:
obtaining a sample of sweat by the device according to claim 1 from deposition of the sample of sweat onto the sweat permeation layer of the device from a finger of the individual;
acquiring a plurality of measurements of a level of the biofluid analyte in sweat of the individual using a self-generated signal or open-circuit voltage from the device;
obtaining, for each of the plurality of measurements of the level of the biofluid analyte in sweat of the individual, a voltage signal without external exertion of a constant voltage or current by discharging via a resistive load between an anode and a cathode of the plurality of electrodes; and
discharging, for each of the plurality of measurements of the level of the biofluid analyte in sweat of the individual, from a biofuel cell of the device, power that is regulated or stored to power electronics that obtain the signal from the plurality of electrodes.
